(19) United States
(12) Patent Application Publication
(10) Pub. No.: US 2009/0313718 A1
(43) Pub. Date: Dec. 17, 2009

(54) POLYNUCLEOTIDES ENCODING CARYOPHYLLENE SYNTHASE AND USES THEREOF

(76) Inventors: Jorg Degenhardt, Naumburg (DE); Tobias Kollner, Grosseutersdorf (DE); Jonathan Gershenzon, Jena (DE)

Correspondence Address: Pearl Cohen Zedek Latzer, LLP, 1500 Broadway, 12th Floor, New York, NY, 10036, US

(21) Appl. No.: 12/373,390

(22) PCT Filed: Jul. 11, 2007
(86) PCT No.: PCT/EP2007/006157

§ 371 Date: Jul. 27, 2009

Related U.S. Application Data
(60) Provisional application No. 60/807,186, filed on Jul. 12, 2006.

(30) Foreign Application Priority Data
Jul. 12, 2006 (EP) 06014509.1

(51) Int. Cl. A01H 1/02; C12N 5/04; C12N 15/63; C07H 21/00; A01H 1/00; A01H 5/00

(52) U.S. Cl. 800/265; 435/419; 435/320.1; 536/23.2; 536/24.1; 800/279; 800/298

(57) ABSTRACT

Described are polynucleotides encoding a caryophyllene synthase. Also described are recombinant nucleic acid molecules and vectors comprising said polynucleotide as well as host cells genetically engineered with said polynucleotides. Further described are caryophyllene synthase polypeptides encoded by said polynucleotide as well as binding molecules specifically recognizing said polypeptide. Additionally, described are transgenic plant cells, plant tissues and plants genetically engineered with the caryophyllene synthase-encoding polynucleotide, corresponding methods for the production of said transgenic plant cells, plant tissues and plants and corresponding uses and methods using said polynucleotide for establishing or enhancing resistance against a herbivores, preferably a root-damaging insects in plants, particularly resistance against the corn rootworm in maize plants. In addition, described are regulatory sequences derived from the caryophyllene synthase-encoding gene described herein and corresponding compounds, methods and uses relating to applications of said regulatory sequence. Moreover, described is a method for breeding a plant having a resistance against a herbivores, preferably a root-damaging insect comprising a selection step for progenitor lines and/or offspring that express said caryophyllene synthase polypeptide.

The present invention relates to polynucleotides encoding a caryophyllene synthase. The present invention furthermore relates to recombinant nucleic acid molecules and vectors comprising said polynucleotide as well as to host cells genetically engineered with said polynucleotides. The present invention also relates to caryophyllene synthase polypeptides encoded by said polynucleotides as well as to binding molecules specifically recognizing said polypeptide. Furthermore, the present invention relates to transgenic plant cells, plant tissues and plants genetically engineered with the caryophyllene synthase-encoding polynucleotide, to corresponding methods for the production of said transgenic plant cells, plant tissues and plants and to corresponding uses and methods using said polynucleotide for establishing or enhancing resistance against herbivores, preferably root-damaging insects in plants, particularly resistance against the corn rootworm in maize plants. In addition, the present invention relates to a regulatory sequence derived from the caryophyllene synthase-encoding gene described herein and to corresponding compounds, methods and uses relating to applications of said regulatory sequence. Moreover, the present invention relates to a method for breeding a plant having a resistance against a herbivore, preferably a root-damaging insect comprising a selection for progenitor lines and/or offspring that express said caryophyllene synthase polypeptide.

Environmentally desirable protection of crops against arthropod herbivores can inter alia use biological control, which depends on the use of antagonists or enemies of the pest organisms. To be effective, it is crucial that the biological control agents are able to find their prey efficiently enough to minimize damage to the crop. More than a decade ago it was discovered that when herbivores feed, the plants produce volatiles that are attractive to the natural enemies of the herbivore (Dicke, Proceedings of Semiochemicals and Pest Control—Prospects for New Applications, 1990, Wageningen, the Netherlands, pp. 3091-3118; and Turlings, Science 250 (1990), 1251-1853). Thus, plants indirectly defend themselves by enhancing the effectiveness of the natural enemies of the herbivores. The use of predators and parasitoids for biological control is receiving more and more attention and for many years it has been common practice in a number of crops in glasshouse as well as open fields (Van Lenteren, In: Biological control: Measures of Success, eds. G. Gurr & S. Wratten, 2000, Kluwer Academic Publishers, Dordrecht; Kfir, Annual Rev. Entomol. 47 (2002), 701-731). Nevertheless, breeders and agronomists have so far paid little attention to the optimisation of biological control. This is probably due to the relatively recent discovery of the phenomenon of indirect defence, the more complex relationships involved and the difficulties associated with quantification of the effects. The beetle Diabrotica virgifera (a species of the corn rootworm) is an agronomically important maize pest in North America that attacks the roots of the plants. It has recently been introduced into Europe and is now quickly spreading within Germany and other European countries. Since the larvae of these beetles live in the soil and damage the roots of the maize plant, it has been very difficult and expensive to control this pest through pesticide applications.

In the work of Rasmann et al. (Nature 434 (2005), 732-737), it was recently demonstrated that some maize varieties are capable to defend themselves against Diabrotica utilizing an indirect defence mechanism. After damage by Diabrotica larvae, the roots of these plants release beta-caryophyllene, a sesquiterpene hydrocarbon, into the soil. This compound attracts nematodes from the surrounding soil that feed on the Diabrotica larvae, eventually killing them. Thereby, the release of caryophyllene leads to an overall benefit to the plants in laboratory experiments. The first field experiments also suggest a reduced Diabrotica damage due to caryophyllene emission.

A similar system is known from the aerial parts of maize plants which, when attacked by caterpillars, release a mixture of odorous compounds that attract parasitic wasps, natural enemies of the herbivores. In a study reported by Degen et al. (Plant Physiol. 135 (2004), 1928-1938), European and American maize inbred lines were examined for their capacity to release these odorous compounds. Thereby, it was found out that European maize varieties are mostly able to produce caryophyllene while almost all American varieties have lost this ability. It is therefore assumed that American varieties are less well defended against Diabrotica due to their inability to produce the caryophyllene signal (Rasmann, loc. cit.). This assumption is currently being tested both in laboratory and field trials.

In view of this, it would be desirable to elucidate the molecular basis of the caryophyllene production in maize plants in order to be able to improve resistance against Diabrotica pest, particularly in North American varieties, more directly.

Thus, the technical problem underlying the present invention is to provide means and methods for enhancing caryophyllene production in the roots of plants in order to increase the plant's resistance against root-damaging insects.

This technical problem is solved by the provision of the embodiments as characterized in the claims.

Accordingly, the present invention relates to polynucleotides selected from the group consisting of
(a) polynucleotides comprising a nucleotide sequence encoding a polypeptide having the amino acid sequence of SEQ ID NO:2 or 4;
(b) polynucleotides comprising the nucleotide sequence shown in SEQ ID NO:1 or 3;
(c) polynucleotides comprising a nucleotide sequence encoding a fragment of the polypeptide encoded by a polynucleotide of (a) or (b), wherein said nucleotide sequence encodes a protein having caryophyllene synthase activity;
(d) polynucleotides comprising a nucleotide sequence the complementary strand of which hybridizes to the polynucleotide of any one of (a) to (c), wherein said nucleotide sequence encodes a protein having caryophyllene synthase activity; and
(e) polynucleotides comprising a nucleotide sequence that deviates from the nucleotide sequence defined in (d) by the degeneracy of the genetic code.

The present invention is based on the isolation of the gene TPS23 encoding a polypeptide having caryophyllene synthase activity from the maize variety Delprim.

This maize variety was shown to synthesize (E)-β-caryophyllene, a sesquiterpene which attracts the entomopathogenic nematode Heterorhabditis megidis which is a predator of the root pest Diabrotica virgifera (Rasmann, loc. cit.).

Thus, it is contemplated that the polynucleotide of the present invention may be used for providing plants that have an increased resistance against root-damaging insects, either by producing corresponding transgenic plants or by applying molecular marker-assisted non-transgenic breeding methods.

In connection with the present invention, the term “resistant” or “resistance” refers to the property of a given plant or plant species to protect itself against an attack by a certain herbivore, preferably a root-damaging insect, whereby said protection may range from a significant reduction to a complete inhibition of herbivory. The type of resistance envisaged in connection with the present invention is based on a so-called tritrophic interaction between a plant, a root-damaging insect and an entomopathogenic nematode (cf. Bouwmeester, Proceedings of BCPC International Congress Crop Science & Technology 2003, 10-12 Nov. 2003, Glasgow, Scotland, Vol 2, 1123-1134). In such a system, the feeding of a herbivore (e.g. a root-damaging insect) leads to the release of one or more volatile compounds (e.g. caryophyllene) which attract one or more predators specialized for the herbivore (e.g. entomopathogenic nematodes). This is described in Rasmann (loc. cit.) for a tritrophic interaction between maize plants, the corn rootworm and the nematode Heterorhabditis megidis. As is apparent from the tritrophic plant defense system on which the present invention is based, the resistance conferred to a plant by expressing the polynucleotide of the invention presupposes that at least one suitable entomopathogenic nematode that is attracted by caryophyllene and that attacks the root-damaging insect is available in the soil surrounding the plant. In order to fulfill this requirement, suitable nematodes may be naturally present in the soil and/or may be added as a biological pest control agent. The nematodes may be added to the soil together with a nutrient suitable for them. As is typical for biological pest control systems, the use of a biological enemy of the pest organism might not lead to the complete elimination of the pest, but will reduce the levels of the pest organism below those resulting in agronomic significant damage. The resulting levels of the pest organism will be dependent on the populations of entomopathogenic nematodes that are either naturally occurring in the soil or will be added to the soil. The already established agronomic procedures of insecticide treatment to reduce the concentration of the adult Diabrotica beetle, soil insecticide treatments to reduce the concentration of the Diabrotica larvae and insecticide treatment of the seeds can be combined with the herein described biological defense strategies.

Successfully established resistance in a plant according to the present invention may be measured by assays described in the prior art, in particular in Rasmann (loc. cit.). Accordingly, a plant produced according to the present invention may be exposed to a root-damaging insect and, subsequently, its capacity to attract entomopathogenic nematodes as compared to a corresponding control plant may be examined. This may be done by using a six-arm olfactometer as disclosed in Rasmann (loc. cit.). In such an assay, a plant according to the present invention shows an attraction of entomopathogenic nematodes which is significantly higher than in the control plant, preferably at least 20%, more preferably at least 50% and most preferably at least 100% higher.

Alternatively, resistance can be detected by measuring the number of herbivores (larvae) around one plant, the number of nematode-infected herbivores around one plant or the number of adult insects emerging around one plant, in comparison to a corresponding control plant. Corresponding assays which were carried out in the field are also described in Rasmann (loc. cit.). Accordingly, with respect to plants of the present invention, the number of herbivores (larvae) around one plant is significantly reduced, preferably by at least 10%, more preferably by at least 20%, as compared to a control plant. Furthermore, the amount of nematode-infected herbivores (larvae) is increased in plants according to the present invention significantly, preferably by at least 100%, more preferably by at least 200% and even more preferably by at least 300% as compared to the control plant. The number of adult insects emerging around one plant is significantly decreased for plants of the present invention as compared to the control plant, preferably by at least 10%, more preferably by at least 20% and even more preferably by at least 30%.

In the context of the present invention, the term “herbivore” relates to insects, in particular insect larvae, that feed on plants.

The term “root-damaging insect” as used herein refers to insect larvae which feed on plant roots. In the framework of the present invention, this term particularly refers to larvae of the genus Diabrotica (interchangeably also referred to herein as “corn rootworm”) and, especially, to larvae of the species Diabrotica virgifera, and more preferably to larvae of the subspecies Diabrotica virgifera virgifera (Western corn rootworm, WCR).

The term “caryophyllene synthase activity” in connection with the present invention refers to the activity of a polypeptide encoded by the polynucleotide of the invention to catalyze the conversion of (E,E)-farnesyl diphosphate (FPP) to (E)-β-caryophyllene.

The activity can be tested for by assays for caryophyllene synthase activity known in the prior art, such as in Cai, Phytochemistry 61 (2002), 523-529, or by assays as described in Examples 2 and 3 of the present application.

Preferably, the caryophyllene synthase of the invention does not accept geranyl diphosphate (GPP) or geranyl-geranyl diphosphate (GGPP) as substrate. Furthermore, the caryophyllene synthase of the invention preferably produces α-humulene and/or δ-elemene as minor side products. It is furthermore preferred that the caryophyllene synthase of the invention has the following biochemical properties:
(i) a catalytic optimum from pH 8.0 to pH 9.5;
(ii) a Kₘ value for FPP of 2.4±0.4 μM;
(iii) a k_{cat} value of 0.0030±0.0002 s⁻¹; and/or
(iv) a requirement for a divalent metal ion cofactor, preferably Mg²⁺ ions at 10 mM (particularly with a Kₘ of 183±34 μM) or Mn²⁺ ions at 0.25 mM (particularly with a Kₘ of 28±6 μM).

As a further preferred characteristic, the polynucleotide of the invention, in particular the caryophyllene synthase gene in its natural source organism, shows inducible expression upon attack of the plant root by a root-damaging insect.

It is furthermore preferred that the expression of the polynucleotide of the invention is not induced upon mechanical lesion of the root or at least only to a significantly smaller degree than in the case of insect infestation.

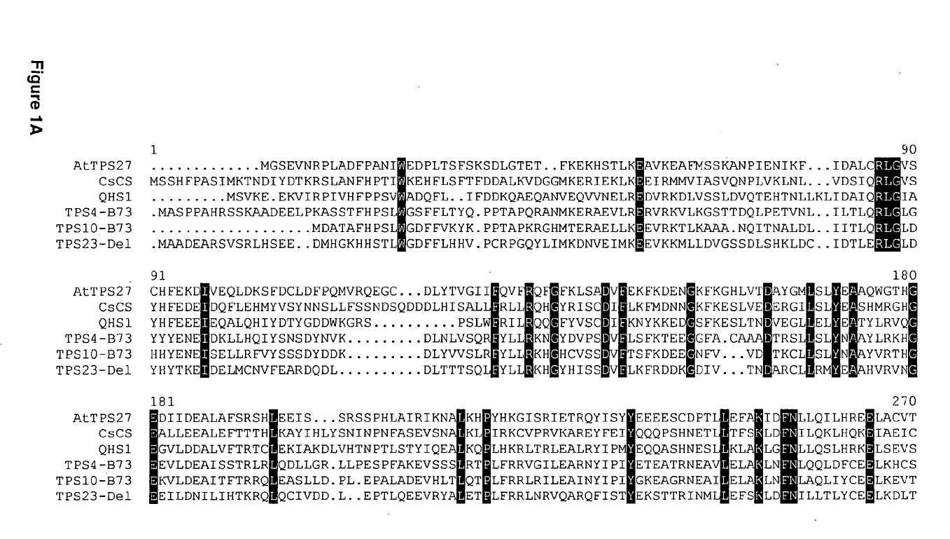

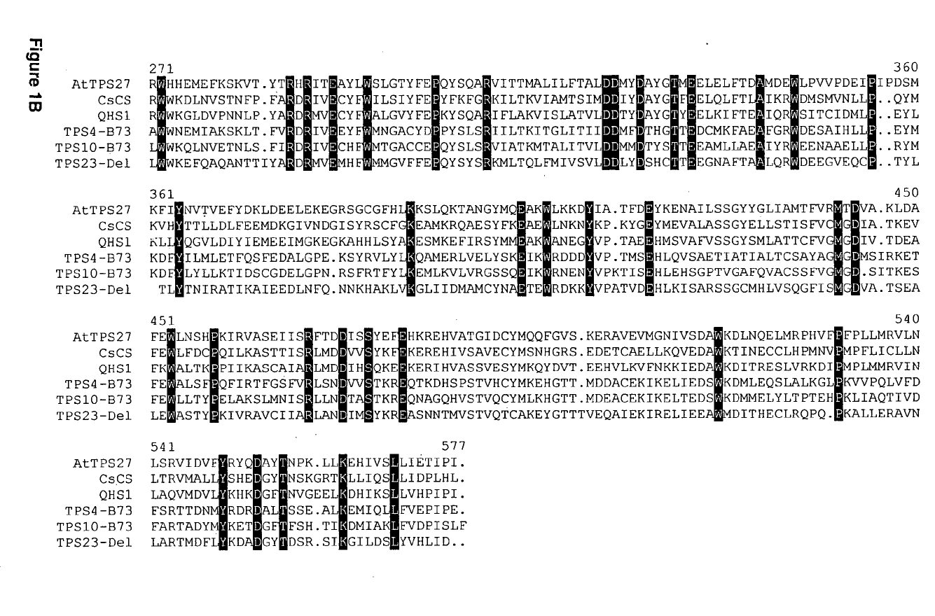

The polynucleotide of the invention preferably comprises the 1.644 bp coding sequence shown in SEQ ID NO: 1 and, particularly preferably, encodes a protein with a predicted molecular mass of 63.6 kDa. As is evident from FIG. 1, numerous amino acids throughout the coding sequence are highly conserved among members of the terpene synthase (TPS) family. The most characteristic element is an Asp-rich DDxxD motif in the C-terminal part of the protein that is involved in the binding of the divalent metal cofactor (Starks et al., 1997). The gene has only a very low amino acid identity to other maize terpene synthases like TPS10 (40.5%, Schnee et al., 2006) and TPS4 (37.8%, Köllner et al., 2004). In the plant material under investigation, no genes with higher sequence identity were found after repeated PCR with maize cDNA and RACE libraries as well as in the maize genomic databases, suggesting that TPS23 is a single gene.

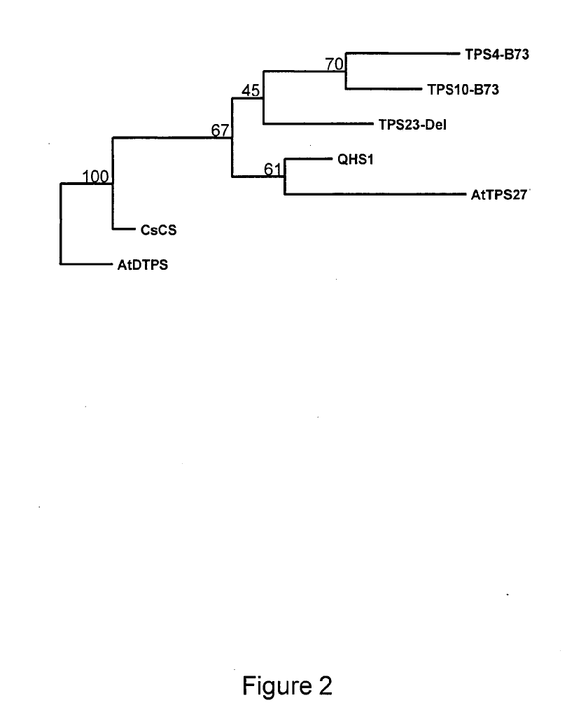

Moreover, TPS23 is the first caryophyllene synthase identified in a monocotyledonous plant. It exhibits a low amino acid identity to caryophyllene synthases known from dicotyledonous plants, i.e. 32.9% with AtTPS27 from Arabidopsis (Aubourg, Mol. Genet. Genomics 267 (2002), 730-745; Chen, Plant Cell 15 (2003), 481-494), 30.3% with CsCS from Cucumis sativus (Mercke, Plant Physiol. 135 (2004), 2012-2024) and 35.1% with QHS1 from Artemisia annua (Cai, Phytochemistry 61 (2002), 523-529). Surprisingly, a dendrogram analysis demonstrates that TPS23 is more closely related to functionally unrelated terpene synthases of maize than to terpene synthases of similar function in other plant species, suggesting a convergent evolution of TPS23 (FIG. 2).

Parts of the coding sequence depicted in SEQ ID NO: 1 were already disclosed in the public maize genome database (http://maize.tigr.org/) in the entries AZM4_{—}53695 and AZM4_{—}20519 (the sequences being depicted herein under SEQ ID NOs:7 and 8, respectively). In addition, in the database Plant GDB, the sequence of a maize genomic fragment was disclosed (AC147506 clone ZMMBBc0299P16) which covers the entire coding sequence (SEQ ID NO: 9). However, it is unlikely that this genomic sequence encodes a functional caryophyllene synthase since the exonic sequences show some errors (nucleotide exchanges and frame-shifts with respect to the nucleotide sequence of SEQ ID NO: 1 and an intron-exon junction not being correct). The nucleotide sequences of SEQ ID NOs: 7 to 9, as well as parts thereof as far as they correspond with the above-described TPS23 coding sequence or functional fragments thereof are excluded from the scope of the present invention.

Up to now, it is not possible to predict, solely based on the amino acid sequence of a terpene synthase, which exact reaction the enzyme catalyzes. Thus, the activity of TPS23 could only be determined by recombinantly expressing the enzyme and assaying the activity in a biochemical test system (Examples 2 and 3).

As indicated above, in addition to polynucleotides comprising a nucleotide sequence which encodes the amino acid sequence of SEQ ID NO: 2 or 4 or comprising the nucleotide sequence of SEQ ID NO: 1 or 3, the present invention also refers to fragments of these polynucleotides as well as to polynucleotides comprising a nucleotide sequence the complementary strand of which hybridizes to these polynucleotides, wherein said fragments and hybridizing polynucleotides encode a protein having caryophyllene synthase activity.

The present invention also relates to polynucleotides which encode a polypeptide, which has a homology, that is to say a sequence identity, of at least 30%, preferably of at least 40%, more preferably of at least 50%, even more preferably of at least 60% and particularly preferred of at least 70%, especially preferred of at least 80% and even more preferred of at least 90% to the entire amino acid sequence as indicated in SEQ ID NO: 2 or 4, the polypeptide having caryophyllene synthase activity.

Moreover, the present invention relates to polynucleotides which encode a polypeptide having caryophyllene synthase activity and the nucleotide sequence of which has a homology, that is to say a sequence identity, of at least 40%, preferably of at least 50%, more preferably of at least 60%, even more preferably of more than 65%, in particular of at least 70%, especially preferred of at least 80%, in particular of at least 90% and even more preferred of at least 95% when compared to the coding sequence shown in SEQ ID NO: 1 or 3.

It is particularly preferred that polynucleotides of the invention that encode a polypeptide having caryophyllene synthase activity show the structural characteristics described above for TPS23.

The present invention also relates to polynucleotides, which encode a polypeptide having caryophyllene synthase activity and the sequence of which deviates from the nucleotide sequences of the above-described polynucleotides due to the degeneracy of the genetic code.

The invention also relates to polynucleotides comprising a nucleotide sequence which is complementary to the whole or a part of one of the above-mentioned sequences.

The polynucleotide of the invention is understood to be isolated. The term “isolated” means that the polynucleotide addressed herein is not in the form as it occurs naturally, if it indeed has a naturally occurring counterpart. Accordingly, the other compounds of the invention described further below are understood to be isolated.

In the context of the present invention the term “hybridization” means hybridization under conventional hybridization conditions, preferably under stringent conditions, as for instance described in Sambrook and Russell (2001), Molecular Cloning: A Laboratory Manual, CSH Press, Cold Spring Harbor, N.Y., USA. In an especially preferred embodiment, the term “hybridization” means that hybridization occurs under the following conditions:

| | |
|---|---|
| Hybridization | 2 × SSC; 10 × Denhardt solution (Fikoll 400 + |
| buffer: | PEG + BSA; ratio 1:1:1); 0.1% SDS; 5 mM EDTA; |
| | 50 mM Na₂HPO₄; 250 μg/ml of herring sperm DNA; |
| | 50 μg/ml of tRNA; or 0.25 M of sodium phosphate |
| | buffer, pH 7.2; 1 mM EDTA 7% SDS |
| Hybridization= | 60° C. |
| temperature T | |
| Washing buffer: | 2 × SSC; 0.1% SDS |
| Washing= | 60° C. |
| temperature T | |

Polynucleotides which hybridize with the polynucleotides of the invention can, in principle, encode a polypeptide having caryophyllene synthase activity from any organism expressing such polypeptides or can encode modified versions thereof.

Polynucleotides which hybridize with the polynucleotides disclosed in connection with the invention can for instance be isolated from genomic libraries or cDNA libraries of bacteria, fungi, plants or animals. Preferably, such polynucleotides are from plant origin, particularly preferred from a plant belonging to the monocotyledons, more preferably from the family of Poaceae and even more preferably from the genus Zea. Preferably, the polynucleotide of the invention is a variant, preferably an ortholog of a polynucleotide comprising SEQ ID NO:1 or 3 and may for example comprise a nucleotide sequence that originates from an agronomically important crop species such as from maize, wheat, barley, oat, rye, rice or sorghum. Alternatively, such polynucleotides can be prepared by genetic engineering or chemical synthesis.

Such hybridizing polynucleotides may be identified and isolated by using the polynucleotides described hereinabove or parts or reverse complements thereof, for instance by hybridization according to standard methods (see for instance Sambrook and Russell (2001), Molecular Cloning: A Laboratory Manual, CSH Press, Cold Spring Harbor, N.Y., USA). Polynucleotides comprising the same or substantially the same nucleotide sequence as indicated in SEQ ID NO: 1 or 3 or parts thereof can, for instance, be used as hybridization probes. The fragments used as hybridization probes can also be synthetic fragments which are prepared by usual synthesis techniques, and the sequence of which is substantially identical with that of a polynucleotide according to the invention.

The molecules hybridizing with the polynucleotides of the invention also comprise fragments, derivatives and allelic variants of the above-described polynucleotides encoding a polypeptide having caryophyllene synthase activity. Herein, fragments are understood to mean parts of the polynucleotides which are long enough to encode the described polypeptide, preferably showing the biological activity of a polypeptide of the invention as described above. In this context, the term derivative means that the sequences of these molecules differ from the sequences of the above-described polynucleotides in one or more positions and show a high degree of homology to these sequences, preferably within the preferred ranges of homology mentioned above.

Preferably, the degree of homology is determined by comparing the respective sequence with the nucleotide sequence of the coding region of SEQ ID NO: 1 or 3. When the sequences which are compared do not have the same length, the degree of homology preferably either refers to the percentage of nucleotide residues in the shorter sequence which are identical to nucleotide residues in the longer sequence or to the percentage of nucleotides in the longer sequence which are identical to nucleotide sequence in the shorter sequence. The degree of homology can be determined conventionally using known computer programs such as the DNASTAR program with the ClustalW analysis. This program can be obtained from DNASTAR, Inc., 1228 South Park Street, Madison, Wis. 53715 or from DNASTAR, Ltd., Abacus House, West Ealing, London W13 0AS UK (support@dnastar.com) and is accessible at the server of the EMBL outstation.

When using the Clustal analysis method to determine whether a particular sequence is, for instance, 80% identical to a reference sequence default settings may be used or the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

Preferably, the degree of homology of the hybridizing polynucleotide is calculated over the complete length of its coding sequence. It is furthermore preferred that such a hybridizing polynucleotide, and in particular the coding sequence comprised therein, has a length of at least 300 nucleotides, preferably at least 500 nucleotides, more preferably of at least 750 nucleotides, even more preferably of at least 1000 nucleotides, and most preferred of at least 1500 nucleotides.

Preferably, sequences hybridizing to a polynucleotide according to the invention comprise a region of homology of at least 90%, preferably of at least 93%, more preferably of at least 95%, still more preferably of at least 98% and particularly preferred of at least 99% identity to an above-described polynucleotide, wherein this region of homology has a length of at least 500 nucleotides, more preferably of at least 750 nucleotides, even more preferably of at least 1000 nucleotides and most preferred of at least 1500 nucleotides.

Homology, moreover, means that there is a functional and/or structural equivalence between the corresponding polynucleotides or polypeptides encoded thereby. Polynucleotides which are homologous to the above-described molecules and represent derivatives of these molecules are normally variations of these molecules which represent modifications having the same biological function. They may be either naturally occurring variations, for instance sequences from other ecotypes, varieties, species, etc., or mutations, and said mutations may have formed naturally or may have been produced by deliberate mutagenesis. Furthermore, the variations may be synthetically produced sequences. The allelic variants may be naturally occurring variants or synthetically produced variants or variants produced by recombinant DNA techniques. Deviations from the above-described polynucleotides may have been produced, e.g., by deletion, substitution, insertion and/or recombination.

The polypeptides encoded by the different variants of the polynucleotides of the invention possess certain characteristics they have in common. These include for instance biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

The biological activity of a polypeptide of the invention, in particular the capacity to synthesize caryophyllene, can be tested in conventional enzyme assays using the substrate of the polypeptide or a suitable modified form thereof.

The polynucleotides of the invention can be DNA molecules, in particular genomic DNA or cDNA. Moreover, the polynucleotides of the invention may be RNA molecules. The polynucleotides of the invention can be obtained for instance from natural sources or may be produced synthetically or by recombinant techniques, such as PCR, and include modified or derivatized nucleic acid molecules as can be obtained by applying techniques described in the pertinent literature.

In a further embodiment, the invention relates to polynucleotides (including oligonucleotides) specifically hybridizing with a polynucleotide of the invention as described above or with the complementary strand thereof. Such polynucleotides have a length of preferably at least 10, in particular at least 15, and particularly preferably of at least 50 nucleotides. Advantageously, their length does not exceed a length of 1000, preferably 500, more preferably 200, still more preferably 100 and most preferably 50 nucleotides. They are characterized in that they specifically hybridize to the polynucleotides of the invention, that is to say that they do not or only to a very minor extent hybridize to nucleotide sequences encoding another terpene synthase or another caryophyllene synthase such as those from dicotyledons described in the prior art.

The polynucleotides of the present embodiment can be used for instance as primers for amplification techniques such as the PCR reaction or as a hybridization probe to isolate related genes. The hybridization conditions and homology values described above in connection with the polynucleotide encoding a polypeptide having caryophyllene synthase activity may likewise apply in connection with the hybridizing polynucleotides mentioned herein.

In another aspect, the present invention relates to recombinant nucleic acid molecules comprising the polynucleotide of the invention described above. The term “recombinant nucleic acid molecule” refers to a nucleic acid molecule which contains in addition to a polynucleotide of the invention as described above at least one further heterologous coding or non-coding nucleotide sequence. The term “heterologous” means that said polynucleotide originates from a different species or from the same species, however, from another location in the genome than said added nucleotide sequence or is synthetic. The term “recombinant” implies that nucleotide sequences are combined into one nucleic acid molecule by the aid of human intervention. The recombinant nucleic acid molecule of the invention can be used alone or as part of a vector.

For instance, the recombinant nucleic acid molecule may encode the polypeptide having caryophyllene synthase activity fused to a marker sequence, such as a peptide, which facilitates purification of the fused polypeptide. The marker sequence may for example be a hexa-histidine peptide, such as the tag provided in a pQE vector (Qiagen, Inc.) or the His tag used in Example 2 (see infra), which provide for convenient purification of the fusion polypeptide. Another suitable marker sequence may be the HA tag which corresponds to an epitope derived from influenza hemagglutinin polypeptide (Wilson, Cell 37 (1984), 767). As a further example, the marker sequence may be glutathione-S-transferase (GST) which, apart from providing a purification tag, enhances polypeptide stability, for instance, in bacterial expression systems.

In a preferred embodiment, the recombinant nucleic acid molecules further comprise expression control sequences operably linked to the polynucleotide comprised in the recombinant nucleic acid molecule. More preferably, these recombinant nucleic acid molecules are expression cassettes.

The term “operatively linked” or “operably linked”, as used throughout the present description, refers to a linkage between one or more expression control sequences and the coding region in the polynucleotide to be expressed in such a way that expression is achieved under conditions compatible with the expression control sequence.

Expression comprises transcription of the heterologous DNA sequence, preferably into a translatable mRNA. Regulatory elements ensuring expression in prokaryotic as well as in eukaryotic cells, preferably in plant cells, are well known to those skilled in the art. They encompass promoters, enhancers, termination signals, targeting signals and the like. Examples are given further below in connection with explanations concerning vectors. In the case of eukaryotic cells, expression control sequences may comprise poly-A signals ensuring termination of transcription and stabilization of the transcript, for example, those of the 35S RNA from Cauliflower Mosaic Virus (CaMV) or the nopaline synthase gene from Agrobacterium tumefaciens. Additional regulatory elements may include transcriptional as well as translational enhancers. A plant translational enhancer often used is the CaMV omega sequences. Similarly, the inclusion of an intron (e.g. intron-1 from the shrunken gene of maize) has been shown to increase expression levels by up to 100-fold (Mait, Transgenic Research 6 (1997), 143-156; Ni, Plant Journal 7 (1995), 661-676).

Moreover, the invention relates to vectors, in particular plasmids, cosmids, viruses, bacteriophages and other vectors commonly used in genetic engineering, which contain the above-described polynucleotides or recombinant nucleic acid molecules of the invention. In a preferred embodiment, the vectors of the invention are suitable for the transformation of fungal cells, cells of microorganisms such as yeast or bacterial cells, animal cells or, in particular, plant cells. In a particularly preferred embodiment such vectors are suitable for stable transformation of plants.

In another preferred embodiment, the vectors further comprise expression control sequences operably linked to said polynucleotides contained in the vectors. These expression control sequences may be suited to ensure transcription and synthesis of a translatable RNA in prokaryotic or eukaryotic cells.

The expression of the polynucleotides of the invention in prokaryotic or eukaryotic cells, for instance in Escherichia coli, is interesting because it permits a more precise characterization of the biological activities of the encoded polypeptide. In particular, it is possible to express these polypeptides in such prokaryotic or eukaryotic cells which are free from interfering polypeptides. In addition, it is possible to insert different mutations into the polynucleotides by methods usual in molecular biology (see for instance Sambrook and Russell (2001), Molecular Cloning: A Laboratory Manual, CSH Press, Cold Spring Harbor, N.Y., USA), leading to the synthesis of polypeptides possibly having modified biological properties. In this regard it is on the one hand possible to produce deletion mutants in which polynucleotides are produced by progressive deletions from the 5′ or 3′ end of the coding DNA sequence, and said polynucleotides lead to the synthesis of correspondingly shortened polypeptides.

On the other hand, the introduction of point mutations is also conceivable at positions at which a modification of the amino acid sequence for instance influences the biological activity or the regulation of the polypeptide.

Moreover, mutants possessing a modified substrate or product specificity can be prepared. Furthermore, it is possible to prepare mutants having a modified activity-temperature-profile. Preferably, such mutants show an increased activity. Alternatively, mutants can be prepared the catalytic activity of which is abolished without loosing substrate binding activity.

Furthermore, in the case of expression in plants, plant tissue or plant cells, the introduction of mutations into the polynucleotides of the invention allows the gene expression rate and/or the activity of the polypeptides encoded by the polynucleotides of the invention to be reduced or increased.

For genetic engineering in prokaryotic cells, the polynucleotides of the invention or parts of these molecules can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, N.Y., USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods.

Additionally, the present invention relates to a method for producing genetically engineered host cells comprising introducing the above-described polynucleotides, recombinant nucleic acid molecules or vectors of the invention into a host cell.

Another embodiment of the invention relates to host cells, in particular prokaryotic or eukaryotic cells, genetically engineered with the above-described polynucleotides, recombinant nucleic acid molecules or vectors of the invention or obtainable by the above-mentioned method for producing genetically engineered host cells, and to cells derived from such transformed cells and containing a polynucleotide, recombinant nucleic acid molecule or vector of the invention. In a preferred embodiment the host cell is genetically modified in such a way that it contains the polynucleotide stably integrated into the genome. Preferentially, the host cell of the invention is a bacterial, yeast, fungus, plant or animal cell.

More preferably the polynucleotide can be expressed so as to lead to the production of a polypeptide having caryophyllene synthase activity. An overview of different expression systems is for instance contained in Methods in Enzymology 153 (1987), 385-516, in Bitter et al. (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440). An overview of yeast expression systems is for instance given by Hensing et al. (Antonie van Leuwenhoek 67 (1995), 261-279), Bussineau et al. (Developments in Biological Standardization 83 (1994), 13-19), Gellissen et al. (Antonie van Leuwenhoek 62 (1992), 79-93, Fleer (Current Opinion in Biotechnology 3 (1992), 486-496), Vedvick (Current Opinion in Biotechnology 2 (1991), 742-745) and Buckholz (Bio/Technology 9 (1991), 1067-1072).

Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), lp1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (deBoer et al., Proc. Natl. Acad. Sci. USA 80 (1983), 21-25). Termination signals for transcription are also described in the literature.

The transformation of the host cell with a polynucleotide or vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc. The polypeptide according to the present invention can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Polypeptide refolding steps can be used, as necessary, in completing configuration of the polypeptide. Finally, high performance liquid chromatography (HPLC) can be employed for final purification steps.

Accordingly, the present invention also relates to a method for the production of a polypeptide encoded by a polynucleotide of the invention as described above in which the above-mentioned host cell is cultivated under conditions allowing for the expression of the polypeptide and in which the polypeptide is isolated from the cells and/or the culture medium.

Moreover, the invention relates to a polypeptide which is encoded by a polynucleotide according to the invention or obtainable by the above-mentioned method for the production of a polypeptide.

The polypeptide of the present invention may, e.g., be a naturally purified product or a product of chemical synthetic procedures or produced by recombinant techniques from a prokaryotic or eukaryotic host (for example, by bacterial, yeast, higher plant, insect and mammalian cells in culture). Depending upon the host employed in a recombinant production procedure, the polypeptide of the present invention may be glycosylated or may be non-glycosylated. The polypeptide of the invention may also include an initial methionine amino acid residue. The polypeptide according to the invention may be further modified to contain additional chemical moieties not normally part of the polypeptide. Those derivatized moieties may, e.g., improve the stability, solubility, the biological half life or absorption of the polypeptide. The moieties may also reduce or eliminate any undesirable side effects of the polypeptide and the like. An overview for these moieties can be found, e.g., in Remington's Pharmaceutical Sciences (18^{th} ed., Mack Publishing Co., Easton, Pa. (1990)). Polyethylene glycol (PEG) is an example for such a chemical moiety which has been used for the preparation of therapeutic polypeptides. The attachment of PEG to polypeptides has been shown to protect them against proteolysis (Sada et al., J. Fermentation Bioengineering 71 (1991), 137-139). Various methods are available for the attachment of certain PEG moieties to polypeptides (for review see: Abuchowski et al., in “Enzymes as Drugs”; Holcerberg and Roberts, eds. (1981), 367-383). Generally, PEG molecules are connected to the polypeptide via a reactive group found on the polypeptide. Amino groups, e.g. on lysines or the amino terminus of the polypeptide are convenient for this attachment among others.

Furthermore, the present invention relates to a binding molecule specifically recognizing the polypeptide of the invention.

The term “specifically recognizing” is meant to refer to the high affinity antibodies or other binding molecules known in the prior art typically have for the target molecule against which they were prepared.

Advantageously, the term “specifically recognizing” refers to a specificity of the binding molecule that allows a distinction between the polypeptide of the invention and related terpene synthase polypeptides, in the sense that the binding molecule does not show a significant cross-reactivity with the latter ones. Said related terpene synthase polypeptides may include the caryophyllene synthases known from dicotyledonous plants and/or other terpene synthases from maize. The person skilled in the art is able to prepare such distinctive binding molecules, for example by selecting non-conserved amino acid stretches, e.g. on the basis of an alignment such as the one shown in FIG. 1, and using them as a starting point (e.g. as an antigen in the case of antibodies) for preparing the binding molecule.

The binding molecule of the present invention may be selected form the group consisting of antibodies, affybodies, trinectins, anticalins, aptamers, RNAs, PNAs and the like, whereby antibodies are preferred.

Based on prior art literature, the person skilled in the art is familiar with obtaining specific binding molecules that may be useful in the methods, kits and uses provided herein. These molecules are directed and bind specifically to or specifically label the polypeptide of the invention described herein. Non-limiting examples of suitable binding molecules may be selected from aptamers (Gold, Ann. Rev. Biochem. 64 (1995), 763-797)), aptazymes, RNAi, shRNA, RNAzymes, ribozymes (see e.g., EP-B1 0 291 533, EP-A1 0 321 201, EP-B1 0 360 257), antisense DNA, antisense oligonucleotides, antisense RNA, siRNA, antibodies (Harlow and Lane “Antibodies, A Laboratory Manual”, CSH Press, Cold Spring Harbor, 1988), affibodies (Hansson, Immunotechnology 4 (1999), 237-252; Henning, Hum Gene Ther. 13 (2000), 1427-1439), lectins, trinectins (Phylos Inc., Lexington, Mass., USA; Xu, Chem. Biol. 9 (2002), 933), anticalins (EPB1 1 017 814) and the like. In accordance with the present invention, the term “aptamer” means nucleic acid molecules that can specifically bind to target molecules. Aptamers commonly comprise RNA, single stranded DNA, modified RNA or modified DNA molecules. The preparation of aptamers is well known in the art and may involve, inter alia, the use of combinatorial RNA libraries to identify binding sites (Gold (1995), Ann. Rev. Biochem 64, 763-797).

A preferred binding molecule in the context of the present invention is an antibody specific for the polypeptide of the present invention.

The antibody useful in context of the present invention can be, for example, polyclonal or monoclonal. The term “antibody” also comprises derivatives or fragments thereof which still retain the binding specificity.

The polypeptide according to the invention, its fragments or other derivatives thereof, or cells expressing them can be used as an immunogen to produce antibodies thereto. The present invention in particular also includes chimeric, single chain, and humanized antibodies, as well as Fab fragments, or the product of an Fab expression library. Various procedures known in the art may be used for the production of such antibodies and fragments.

In context of the present invention, the term “antibody” relates to full immunoglobulin molecules as well as to parts of such immunoglobulin molecules substantially retaining binding specificity. Furthermore, the term relates, as discussed above, to modified and/or altered antibody molecules, like chimeric and humanized antibodies. The term also relates to recombinantly or synthetically generated/synthesized antibodies. The term also relates to intact antibodies as well as to antibody fragments thereof, like, separated light and heavy chains, Fab, Fab/c, Fv, Fab′, F(ab′)₂. The term “antibody” also comprises bifunctional antibodies, trifunctional antibodies and antibody constructs, like single chain Fvs (scFv) or antibody-fusion proteins.

Techniques for the production of antibodies are well known in the art and described, e.g. in Harlow and Lane “Antibodies, A Laboratory Manual”, CSH Press, Cold Spring Harbor, 1988. Antibodies directed against a polypeptide according to the present invention can be obtained, e.g., by direct injection of the polypeptide into an animal or by administering the polypeptide to an animal, preferably a non-human animal. The antibody so obtained will then bind the polypeptide itself. In this manner, even a sequence encoding only a fragment of the polypeptide can be used to generate antibodies binding the whole native polypeptide.

Particularly preferred in the context of the present invention are monoclonal antibodies. For the preparation of monoclonal antibodies, any technique which provides antibodies produced by continuous cell line cultures can be used. Examples for such techniques include the hybridoma technique (Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Various procedures are known in the art and may be used for the production of such antibodies and/or fragments. Thus, the antibody derivatives can also be produced by peptidomimetics. Further, techniques described for the production of single chain antibodies (see, inter alia, U.S. Pat. No. 4,946,778) can be adapted to produce single chain antibodies specifically recognizing the polypeptide of the invention. Also, transgenic animals may be used to express humanized antibodies to the polypeptide of the invention.

Surface plasmon resonance as employed in the BIAcore system can be used to increase the efficiency of phage antibodies which bind to an epitope of the polypeptide of the invention (Schier, Human Antibodies Hybridomas 7 (1996), 97-105; Malmborg, J. Immunol. Methods 183 (1995), 7-13). Accordingly, also phage antibodies can be used in context of this invention.

Binding molecules according to the invention can be used for detecting the presence, absence or amount of the polypeptide of the invention in a sample, in particular in the framework of methods and uses described herein further below. The binding molecules may furthermore be used for isolating the polypeptide from a biological source material or for detecting the polypeptide in a sample.

Furthermore, the present invention relates to a method for producing a transgenic plant, plant cell or plant tissue comprising the introduction of at least one of the above-described polynucleotides, recombinant nucleic acid molecules or vectors of the invention into the genome of a plant, plant cell or plant tissue.

Preferably, said method comprises (a) the introduction of at least one of said polynucleotides, recombinant nucleic acid molecules or vectors into the genome of a plant cell and regenerating the cell of (a) to a transgenic plant or transgenic plant tissue. Optionally, the method may further comprise step (c) producing progeny from the plants produced in step (b).

In one aspect, the present invention accordingly refers to transgenic plant cells which are genetically engineered with at least one of the polynucleotides, recombinant nucleic acid molecules or vectors of the invention described above or of transgenic plant cells which are obtainable by the aforementioned method for producing a transgenic plant cell.

In a further aspect, the invention relates to transgenic plants or plant tissue comprising plant cells which are genetically engineered with the polynucleotide of the invention or which contain the recombinant nucleic acid molecule or the vector of the invention or to transgenic plants obtainable by the method mentioned above. Preferably, in the transgenic plant of the invention, the polynucleotide of the invention is expressed at least in one part, i.e. organ, tissue or cell type, of the plant. Preferably, this expression leads to an increase of caryophyllene synthase activity in the cells or tissues which express said polynucleotide. Increase of activity can be detected for instance by measuring the amount of transcript and/or protein in the transformed cell, tissue or plant in comparison to corresponding measurements at non-transformed plant cells, tissue or plants. According to the teachings of the present invention, an increase of the activity of the polypeptide of the invention in transgenic plants leads to an increase of resistance against a root-damaging insect to which a corresponding wild-type plant is susceptible or at least more susceptible.

The term “increased activity” refers to a significant increase of the caryophyllene synthase activity of the polypeptide of the invention in the transgenic plant compared to a corresponding wild-type plant. Preferably, said activity is increased in the transgenic plant by at least 10%, preferably by at least 20%, more preferably by at least 50%, and even more preferred by at least 100% as compared to the corresponding wild-type plant. It is even more preferred that the corresponding wild-type plant shows no detectable caryophyllene synthase activity as opposed to a significant caryophyllene synthase activity observable in the transgenic plant of the invention. Caryophyllene synthase activity may be determined by the use of enzyme assays using a preparation from a plant sample by applying suitable methods known in the art or described herein.

An increase of the activity of the polypeptide of the invention may also be inferred from a significant increase of the amount of corresponding transcript and/or protein present in the transgenic plant. Preferentially, transgenic plants having an increased activity of the polypeptide of the invention may be characterized by an increase of the amount of transcript corresponding to the polynucleotide of the invention by at least 20%, preferably at least 50% and more preferably at least 100% as compared to the corresponding wild-type plant. Likewise, it is preferred that transgenic plants having an increased activity of the polypeptide of the invention may be characterized by an increase of the protein amount of the polypeptide of the invention by at least 20%, preferably at least 50% and more preferably at least 100% as compared to the corresponding wild-type plant. Most preferably, the corresponding wild-type plant shows no detectable amount of such transcript and/or such protein as opposed to significant transcript and/or protein amounts observable in the transgenic plant of the invention.

The polynucleotide introduced into the transgenic plant can in principle be expressed in all or substantially all cells of the plant. However, it is also possible that it is only expressed in certain parts, organs, cell types, tissues etc., provided that expression in the root is ensured. Moreover, it is possible that expression of the polynucleotide only takes place upon induction, at a certain developmental stage or, as it is preferred, upon induction by attack of the root by a root-damaging insect. In a preferred embodiment, the polynucleotide is expressed in those parts of the plant that are exposed to pathogen attack, for example the rhizodermis.

In order to be expressed, the polynucleotide that is introduced into a plant cell is preferably operatively linked to one or more expression control sequences, e.g. a promoter, active in this plant cell. Suitable promoter sequences are known to the skilled person and are described in the pertinent literature.

The promoter may be homologous or heterologous with regard to its origin and/or with regard to the gene to be expressed. Suitable promoters are for instance the promoter of the 35S RNA of the Cauliflower Mosaic Virus (see for instance U.S. Pat. No. 5,352,605) and the ubiquitin-promoter (see for instance U.S. Pat. No. 5,614,399) which lend themselves to constitutive expression. However, promoters which are only activated at a point in time determined by external influences can also be used (see for instance WO 93/07279). In this connection, promoters of heat shock proteins which permit simple induction may be of particular interest. Likewise, artificial and/or chemically inducible promoters may be used in this context. In one embodiment, promoters which ensure constitutive expression are preferred. However, in another preferred embodiment, the polynucleotide may be operatively linked to a promoter which is inducible upon attack by a root-damaging insect. Even more preferably, this promoter is additionally specific for expression in the root. Accordingly, a particularly preferred promoter is or comprises the regulatory sequence of the present invention, which is described in detail further below.

Moreover, the polynucleotide may be linked to a termination sequence which serves to terminate transcription correctly and to add a poly-A-tail to the transcript which is believed to have a function in the stabilization of the transcripts. Such elements are described in the literature (see for instance Gielen et al., EMBO J. 8 (1989), 23-29) and can be replaced at will.

Furthermore, if needed, polypeptide expression can in principle be targeted to any sub-localization of plant cells (e.g. cytosol, plastids, vacuole, mitochondria) or the plant (e.g. apoplast). In order to achieve the localization in a particular compartment, the coding region to be expressed may be linked to DNA sequences encoding a signal sequence (also called “transit peptide”) ensuring localization in the respective compartment. It is evident that these DNA sequences are to be arranged in the same reading frame as the coding region to be expressed. Preferred in connection with the present invention is the expression of the polynucleotide of the invention into the cytosol and/or the endoplasmatic reticulum since this is presumed to be the site of sesquiterpene synthesis in plant cells (Chen, Plant Cell 15 (2003), 481-494).

In order to ensure the location in the plastids, it is conceivable to use one of the following transit peptides: of the plastidic Ferredoxin: NADP+ oxidoreductase (FNR) of spinach which is enclosed in Jansen et al. (Current Genetics 13 (1988), 517-522). In particular, the sequence ranging from nucleotides −171 to 165 of the cDNA sequence disclosed therein can be used which comprises the 5′ non-translated region as well as the sequence encoding the transit peptide. Another example is the transit peptide of the waxy protein of maize including the first 34 amino acid residues of the mature waxy protein (Klosgen et al., Mol. Gen. Genet. 217 (1989), 155-161). It is also possible to use this transit peptide without the first 34 amino acids of the mature protein. Furthermore, the signal peptides of the ribulose bisphosphate carboxylase small subunit (Wolter et al., Proc. Natl. Acad. Sci. USA 85 (1988), 846-850; Nawrath et al., Proc. Natl. Acad. Sci. USA 91 (1994), 12760-12764), of the NADP malate dehydrogenase (Gallardo et al., Planta 197 (1995), 324-332), of the glutathione reductase (Creissen et al., Plant J. 8 (1995), 167-175) or of the R1 protein (Lorberth et al. Nature Biotechnology 16, (1998), 473-477) can be used.

In order to ensure the location in the vacuole, it is conceivable to use one of the following transit peptides: the N-terminal sequence (146 amino acids) of the patatin protein (Sonnewald et al., Plant J. 1 (1991), 95-106) or the signal sequences described by Matsuoka and Neuhaus (Journal of Experimental Botany 50 (1999), 165-174); Chrispeels and Raikhel (Cell 68 (1992), 613-616); Matsuoka and Nakamura (Proc. Natl. Acad. Sci. USA 88 (1991), 834-838); Bednarek and Raikhel (Plant Cell 3 (1991), 1195-1206); and Nakamura and Matsuoka (Plant Phys. 101 (1993), 1-5).

In order to ensure the localization in the mitochondria, it is for example conceivable to use the transit peptide described by Braun (EMBO J. 11, (1992), 3219-3227).

In order to ensure the localization in the apoplast, it is conceivable to use one of the following transit peptides: signal sequence of the proteinase inhibitor II-gene (Keil et al., Nucleic Acid Res. 14 (1986), 5641-5650; von Schaewen et al., EMBO J. 9 (1990), 30-33), of the levansucrase gene from Erwinia amylovora (Geier and Geider, Phys. Mol. Plant Pathol. 42 (1993), 387-404), of a fragment of the patatin gene B33 from Solanum tuberosum, which encodes the first 33 amino acids (Rosahl et al., Mol Gen. Genet. 203 (1986), 214-220) or of the one described by Oshima et al. (Nucleic Acid Res. 18 (1990), 181).

The transgenic plants of the invention may, in principle, be plants of any plant species. They may be both monocotyledonous and dicotyledonous plants. Preferably, the plants are useful plants, i.e. commercially important plants, cultivated by man for nutrition or for technical, in particular industrial, purposes. They may be sugar storing and/or starch-storing plants, for instance cereal species (rye, barley, oat, wheat, maize, millet, sago etc.), rice, pea, marrow pea, cassava, sugar cane, sugar beet and potato; tomato, rape, soybean, hemp, flax, sunflower, cow pea or arrowroot, fiber-forming plants (e.g. flax, hemp, cotton), oil-storing plants (e.g. rape, sunflower, soybean) and protein-storing plants (e.g. legumes, cereals, soybeans). The plants within the scope of the invention also include fruit trees, palms and other trees or wooden plants being of economical value such as in forestry. Moreover, the method of the invention relates to forage plants (e.g. forage and pasture grasses, such as alfalfa, clover, ryegrass) and vegetable plants (e.g. tomato, lettuce, chicory) and ornamental plants (e.g. roses, tulips, hyacinths). Preferably, the transgenic plant of the invention is a monocotyledonous plant, more preferably a plant of the Poaceae family, particularly preferably a wheat, barley, oat, rye, rice or sorghum plant. Most preferred are transgenic plants being maize.

According to the provisions of the invention, transgenic plants can be prepared by introducing a polynucleotide into plant cells and regenerating the transformed cells to plants by methods well known to the person skilled in the art.

Methods for the introduction of foreign genes into plants are also well known in the art. These include, for example, the transformation of plant cells or tissues with T-DNA using Agrobacterium tumefaciens or Agrobacterium rhizogenes, the fusion of protoplasts, direct gene transfer (see, e.g., EP-A 164 575), injection, electroporation, vacuum infiltration, biolistic methods like particle bombardment, pollen-mediated transformation, plant RNA virus-mediated transformation, liposome-mediated transformation, transformation using wounded or enzyme-degraded immature embryos, or wounded or enzyme-degraded embryogenic callus and other methods known in the art. The vectors used in the method of the invention may contain further functional elements, for example “left border”- and “right border”-sequences of the T-DNA of Agrobacterium which allow stable integration into the plant genome. Furthermore, methods and vectors are known to the person skilled in the art which permit the generation of marker free transgenic plants, i.e. the selectable or scorable marker gene is lost at a certain stage of plant development or plant breeding. This can be achieved by, for example co-transformation (Lyznik, Plant Mol. Biol. 13 (1989), 151-161; Peng, Plant Mol. Biol. 27 (1995), 91-104) and/or by using systems which utilize enzymes capable of promoting homologous recombination in plants (see, e.g., WO97/08331; Bayley, Plant Mol. Biol. 18 (1992), 353-361); Lloyd, Mol. Gen. Genet. 242 (1994), 653-657; Maeser, Mol. Gen. Genet. 230 (1991), 170-176; Onouchi, Nucl. Acids Res. 19 (1991), 6373-6378). Methods for the preparation of appropriate vectors are described by, e.g., Sambrook and Russell (2001), Molecular Cloning: A Laboratory Manual, CSH Press, Cold Spring Harbor, N.Y., USA.

Suitable strains of Agrobacterium tumefaciens and vectors as well as transformation of Agrobacteria and appropriate growth and selection media are well known to those skilled in the art and are described in the prior art (GV3101 (μMK9ORK), Koncz, Mol. Gen. Genet. 204 (1986), 383-396; C58C1 (pGV 3850kan), Deblaere, Nucl. Acid Res. 13 (1985), 4777; Bevan, Nucleic. Acid Res. 12(1984), 8711; Koncz, Proc. Natl. Acad. Sci. USA 86 (1989), 8467-8471; Koncz, Plant Mol. Biol. 20 (1992), 963-976; Koncz, Specialized vectors for gene tagging and expression studies. In: Plant Molecular Biology Manual Vol 2, Gelvin and Schilperoort (Eds.), Dordrecht, The Netherlands: Kluwer Academic Publ. (1994), 1-22; EP-A-120 516; Hoekema: The Binary Plant Vector System, Offsetdrukkerij Kanters B. V., Alblasserdam (1985), Chapter V, Fraley, Crit. Rev. Plant. Sci., 4, 1-46; An, EMBO J. 4 (1985), 277-287). Although the use of Agrobacterium tumefaciens is preferred in the method of the invention, other Agrobacterium strains, such as Agrobacterium rhizogenes, may be used, for example if a phenotype conferred by said strain is desired.

Methods for the transformation using biolistic methods are well known to the person skilled in the art; see, e.g., Wan, Plant Physiol. 104 (1994), 37-48; Vasil, Bio/Technology 11 (1993), 1553-1558 and Christou (1996) Trends in Plant Science 1, 423-431. Microinjection can be performed as described in Potrykus and Spangenberg (eds.), Gene Transfer To Plants. Springer Verlag, Berlin, N.Y. (1995).

The transformation of most dicotyledonous plants is possible with the methods described above. But also for the transformation of monocotyledonous plants several successful transformation techniques have been developed. These include the transformation using biolistic methods as, e.g., described above as well as protoplast transformation, electroporation of partially permeabilized cells, introduction of DNA using glass fibers, etc. Also, the transformation of monocotyledonous plants by means of Agrobacterium-based vectors has been described (Chan et al., Plant Mol. Biol. 22 (1993), 491-506; Hiei et al., Plant J. 6 (1994) 271-282; Deng et al, Science in China 33 (1990), 28-34; Wilmink et al, Plant Cell Reports 11 (1992), 76-80; May et al., Bio/Technology 13 (1995), 486-492; Conner and Dormisse, Int. J. Plant Sci. 153 (1992), 550-555; Ritchie et al. Transgenic Res. 2 (1993), 252-265). An alternative system for transforming monocotyledonous plants is the transformation by the biolistic approach (Wan and Lemaux, Plant Physiol. 104 (1994), 37-48; Vasil et al., Bio/Technology 11 (1993), 1553-1558; Ritala et al., Plant Mol. Biol. 24 (1994) 317-325; Spencer et al., Theor. Appl. Genet. 79 (1990), 625-631). The transformation of maize in particular has been repeatedly described in the literature (see for instance WO 95/06128, EP 0 513 849, EP 0 465 875, EP 29 24 35; Fromm et al, Biotechnology 8, (1990), 833-844; Gordon-Kamm et al., Plant Cell 2, (1990), 603-618; Koziel et al., Biotechnology 11 (1993), 194-200; Moroc et al., Theor. Appl. Genet. 80, (1990), 721-726; and, preferably, The Maize Handbook. Eds. M. Freeling and V. Walbot, Springer Verlag New York, 1996). The successful transformation of other types of cereals has also been described for instance of barley (Wan and Lemaux, supra; Ritala et al., supra, Krens et al., Nature 296 (1982), 72-74), wheat (Nehra et al., Plant J. 5 (1994), 285-297) and rice.

The resulting transformed plant cell can then be used to regenerate a transformed plant in a manner known by a skilled person.

In addition, the present invention relates to transgenic plants which show an increased activity of the polypeptide encoded by the polynucleotide the invention compared to a corresponding wild-type plant.

In this context, the “increased activity” complies with the definition stated above and it can be determined accordingly.

Corresponding increases of the activity of the polypeptide of the invention may for instance be achieved by expressing said polynucleotide in cells of a transgenic plant from a heterologous construct for example as described above. However, the state of the art provides further methods for achieving a corresponding increased activity. For example, the endogenous gene encoding the caryophyllene synthase of the invention may be modified accordingly at its natural location, e.g. by homologous recombination. In particular, the promoter of this gene can for instance be altered in a way that promoter activity is enhanced. In the alternative, the coding region of the gene can be modified so that the encoded polypeptide shows an increased activity, e.g. by specifically substituting amino acid residues so as to re-establish the amino acid sequence encoded by an active allele, such as the alleles characterized by the coding sequences of SEQ ID Nos. 1 and 3. Applicable homologous recombination techniques (also known as “in vivo mutagenesis”) are known to the person skilled in the art and are described in the literature. One such technique involves the use of a hybrid RNA-DNA oligonucleotide (“chimeroplast”) which is introduced into cells by transformation (TIBTECH 15 (1997), 441-447; WO95/15972; Kren, Hepatology 25 (1997), 1462-1468; Cole-Strauss, Science 273 (1996), 1386-1389). Thereby, part of the DNA component of the RNA-DNA oligonucleotide is homologous with the target gene sequence, however, displays in comparison to this sequence a mutation or a heterologous region which is surrounded by the homologous regions. The term “heterologous region” refers to any sequence that can be introduced and which is different from that to be modified. By means of base pairing of the homologous regions with the target sequence followed by a homologous recombination, the mutation or the heterologous region contained in the DNA component of the RNA-DNA oligonucleotide can be transferred to the corresponding gene. By means of in vivo mutagenesis, any part of the gene encoding the caryophyllene synthase of the invention can be modified as long as it results in an increase of the activity of this protein.

In a preferred embodiment, the above-described transgenic plants show, upon an increased activity of the protein encoded by the polynucleotide of the invention, an increased resistance against a herbivore, preferably a root-damaging insect to which a corresponding wild-type plant is more susceptible.

The term “increased resistance” may refer both to an enhancement of a resistance already present in the wild-type plant and to the establishment of a resistance that is not present in the wild-type plant.

Preferably, these transgenic plants contain a polynucleotide as defined above, i.e. a polynucleotide or a recombinant nucleic acid molecule that is introduced in a plant cell and the presence of which in the genome of said plant preferably leads to an increased activity of the caryophyllene synthase of the invention, stably integrated into the genome.

The invention also relates to propagation material of the transgenic plants of the invention, said material comprising plant cells according to the invention. The term “propagation material” comprises those components or parts of the plant which are suitable to produce offspring vegetatively or generatively. Suitable means for vegetative propagation are for instance cuttings, callus cultures, rhizomes or tubers. Other propagation material includes for instance fruits, seeds, seedlings, protoplasts, cell cultures etc. The preferred propagation materials are tubers and seeds.

The invention also relates to harvestable parts of the plants of the invention such as, for instance, fruits, seeds, tubers, rootstocks, leaves or flowers.

In accordance with the above explanations, the invention furthermore relates to a method for conferring resistance or increased resistance against a herbivore, preferably a root-damaging insect, to a plant comprising the step of providing a transgenic plant in which the activity of the polypeptide encoded by the above-described polynucleotide of the invention is increased as compared to a corresponding wild-type plant.

Also within the scope of the invention is the use of the polynucleotide, of the recombinant nucleic acid molecule, of the vector, of the host cell, of the polypeptide, of the binding molecule or of the transgenic plant of the invention, said matters being described above in detail, for establishing or enhancing resistance against a herbivore, preferably a root-damaging insect, in plants.

In a preferred embodiment of the above-mentioned method and the above-mentioned use, the plants are maize plants and the root-damaging insect is the corn rootworm.

In accordance with another aspect of the present invention, it was discovered that the regulatory sequence of the caryophyllene synthase gene of the invention is inducible upon attack of the root by a root-damaging insect. Likewise, the regulatory sequence is inducible upon attack of above-ground organs, such as leaves, by insect larvae. Accordingly, the invention further relates to a regulatory sequence which comprises a DNA sequence selected from the group consisting of
(a) the DNA sequence shown in SEQ ID NO: 5 or 6;
(b) fragments of the DNA sequence of (a) being capable of mediating the transcription of a coding sequence operably linked thereto; and
(c) DNA sequences the complementary strand of which hybridizes to the DNA sequence of (a) or (b), wherein said DNA sequence is capable of mediating the transcription of a coding sequence operably linked thereto.

In a preferred embodiment, the regulatory sequence of the invention comprises a promoter sequence from a gene comprising, as its coding region, a caryophyllene synthase-encoding polynucleotide of the invention, as described above.

The regulatory sequence of the present invention is capable of mediating transcription of a coding sequence operably linked thereto, wherein the transcription is inducible upon attack by a herbivore, preferably the attack is directed to a plant root by a root-damaging insect as defined above. Preferably, the inducible transcription is tissue-specific, in particular root-specific. The transcription-mediating activity may be assayed by methods known to the person skilled in the art, including in particular the use of promoter-reporter gene constructs in transgenic plants. Examples of suitable reporter genes include luciferase, green fluorescent protein (GFP) and GUS.

In the context with the present invention, the term “regulatory sequence” refers to sequences which influence the specificity and/or level of expression, for example in the sense that they confer inducibility and/or cell and/or tissue specificity. Such regions can be located upstream of the transcription initiation site, but can also be located downstream of it, e.g., in transcribed but not translated leader sequences. Accordingly, the term “mediating the transcription of a coding sequence operably linked thereto” refers to the above-mentioned capability of the regulatory sequence to influence the specificity and/or level of expression. The regulatory sequence of the invention may comprise one or more regulatory elements of a promoter or it may be a promoter itself. “Regulatory element” is intended to refer to DNA sequences which on their own have no promoter activity, and require further elements such as CAAT and TATA box in operable linkage so as to form a functional promoter. For example, a regulatory element may become functional when it is combined with a minimal promoter, such as the CaMV 35S, the CHS or other minimal plant promoters well-known in the art.

The term “promoter”, within the meaning of the present invention refers to nucleotide sequences necessary for transcription initiation, i.e. RNA polymerase binding, and may also include, for example, the TATA box.

The term “inducible” means that the transcription rate is significantly increased upon attack by a root-damaging insect as compared to the state of the same cell, tissue or organ before that event. Preferably, this increase is at least 2-fold, more preferably at least 5-fold, further preferably at least 10-fold and, most preferably, there is no detectable transcription before the attack in said cell, tissue or organ.

The term “tissue-specific” is intended to refer to a significant higher level of transcription in the tissue in question as compared to other tissues of the same plant. Preferably, the level of transcription in the tissue in question is at least 2-fold, more preferably at least 5-fold and even more preferably at least 10-fold higher than in other tissues of the same plant. Most preferably, there is no detectable transcription in said other tissues as opposed to the tissue in question.

The above-said is correspondingly applicable to organ-specificity and root-specificity in relation to other organs of the same plant.

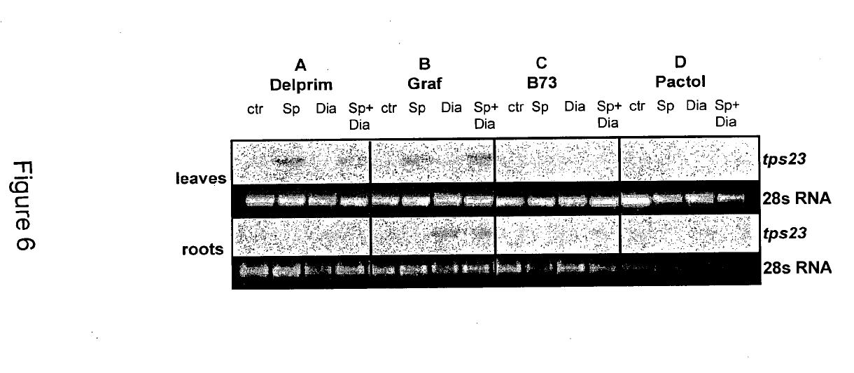

As is evident from the results reported in appended Example 4 and FIG. 6, the TPS23 gene disclosed herein not only shows Diabrotica-induced transcription in the root, but also induced transcription in leaves upon attack by Spodoptera (a caterpillar feeding on leaves. Thus, it is contemplated that the regulatory sequence of the invention may include at least one further inducibility/tissue-specificity characteristic in addition to the root-specific inducibility upon root-damaging insect attack.

The inducibility and tissue-specificity properties can be tested for a given regulatory sequence by methods known to the person skilled in the art. These include the detection of transcripts by way of Northern blot hybridization or RT-PCR as well as the use of promoter-reporter gene constructs in transgenic plants.

Examples of suitable reporter genes include luciferase, green fluorescent protein (GFP) and GUS.

The term “gene” as referred to above relates to caryophyllene synthase genes which comprise as their coding sequence the polynucleotide of the invention encoding said enzyme. In such a gene, the coding sequence may be interrupted by introns. Thus, the regulatory sequence of the invention may be obtained from genes or alleles homologous to the TPS23 gene disclosed herein, said genes or alleles being present in other maize varieties or other plant species.

The caryophyllene synthase genes from which a regulatory sequence according to the invention can be isolated can be selected by examining the inducibility and tissue-specificity properties mentioned above in connection with the regulatory sequence of the invention by applying suitable techniques.

In a preferred embodiment, the regulatory sequence of the invention comprises the sequence shown in SEQ ID NO: 5 or 6. Furthermore, a regulatory sequence of the invention can comprise a fragment of the DNA sequence shown in SEQ ID NO: 5 or 6 which is capable of mediating the transcription of a coding sequence operably linked thereto.

Moreover, the present invention relates to regulatory sequences comprising a DNA sequence the complementary strand of which hybridizes to a DNA sequence as mentioned in sections (a) to (b), above, wherein said DNA sequence is capable of mediating transcription of a coding sequence operably linked thereto.

The present invention also relates to regulatory sequences comprising a DNA a sequence of which has a homology, that is to say a sequence identity, of at least 40%, preferably of at least 50%, more preferably of at least 60%, even more preferably of more than 65%, in particular of at least 70%, especially preferred of at least 80%, in particular of at least 90% and even more preferred of at least 95% when compared to the coding region of the DNA sequence shown in SEQ ID NO: 5 or 6, wherein said DNA sequence is capable of mediating transcription of a coding sequence operably linked thereto.

The invention also relates to polynucleotides comprising a nucleotide sequence which is complementary to the whole or a part of one of the above-mentioned regulatory sequences.

In the context of the present embodiment, the term “hybridization” has the meaning as defined further above. Likewise, the explanations concerning homology and sequence identity stated above apply herein as well.

DNA sequences which hybridize with the regulatory sequences disclosed in connection with the invention can for instance be isolated from genomic libraries of any suitable organism. Preferably, such polynucleotides are from plant origin, particularly preferred from a monocotyledonous plant, more preferably from a plant of the family of Poaceae.

Preferably, the regulatory sequence of the invention is a variant, preferably an ortholog of a regulatory comprising SEQ ID NO: 5 or 6 and may for example comprise a DNA sequence that originates from an agronomically important crop species such as from maize, wheat, barley, oat, rye, rice or sorghum. Alternatively, such polynucleotides can be prepared by genetic engineering or chemical synthesis. The regulatory sequence of the invention may be a naturally occurring promoter or a functional fragment thereof or a regulatory element isolated therefrom capable of mediating transcription of a coding sequence operably linked thereto. Likewise, the regulatory sequence of the invention may be synthetic or may be a chimeric construct, in particular a chimeric promoter, into which for example one or more further functional elements have been introduced following the individual aims and needs.

The invention also relates to polynucleotides (including oligonucleotides) specifically hybridizing with a regulatory sequence of the invention or with the complementary strand thereof. Such polynucleotides have a length of preferably at least 10, in particular at least 15, and particularly preferably of at least 50 nucleotides. Advantageously, their length does not exceed a length of 1000, preferably 500, more preferably 200, still more preferably 100 and most preferably 50 nucleotides. They are characterized in that they specifically hybridize to the regulatory sequences of the invention, that is to say that they do not or only to a very minor extent hybridize to other nucleotide sequences. The oligonucleotides of the invention can be used for instance as primers for amplification techniques such as the PCR reaction or as a hybridization probe to isolate related genes or regulatory sequences. The hybridization conditions and homology values described above in connection with the polynucleotide of the invention encoding a polypeptide having caryophyllene synthase activity may likewise apply in connection with the hybridizing polynucleotides mentioned herein.

In another aspect, the present invention relates to recombinant nucleic acid molecules comprising the regulatory sequence of the invention described above. The definition of the term “recombinant nucleic acid molecule” given above applies herein accordingly.

In a preferred embodiment, the recombinant nucleic acid molecules further comprise a heterologous coding sequence operably linked to the regulatory sequence of the invention. More preferably, these recombinant nucleic acid molecules are expression cassettes. In this context, the term “operatively linked” refers to a linkage between the regulatory sequence and the coding sequence in such a way that expression is achieved under conditions compatible with the regulatory sequence.

Expression comprises transcription of the heterologous coding sequence, preferably into a translatable mRNA. The recombinant nucleic acid molecule may comprise further regulatory elements, as are well known to those skilled in the art, such as enhancers, termination signals, targeting signals and the like. Examples are given above in connection with explanations concerning vectors. Such further expression control sequences may comprise poly-A signals ensuring termination of transcription and stabilization of the transcript, for example, those of the 35S RNA from Cauliflower Mosaic Virus (CaMV) or the nopaline synthase gene from Agrobacterium tumefaciens. Additional regulatory elements may include transcriptional as well as translational enhancers. A plant translational enhancer often used is the CaMV omega sequences. Similarly, the inclusion of an intron (e.g. intron-1 from the shrunken gene of maize) has been shown to increase expression levels by up to 100-fold (Mait, Transgenic Research 6 (1997), 143-156; Ni, Plant Journal 7 (1995), 661-676).

The heterologous coding sequence may be any desired one. Preferentially, it is one which makes use of the specific properties of the regulatory sequence of the invention described above.

In a preferred embodiment, the heterologous coding sequence is a reporter gene. One preferred application of such a recombinant nucleic acid molecules could be to transform plants therewith and to use such plants as biological indicators for the occurrence of root-damaging insects in the soil.

A positive reaction readily visible for example through the dying of the plant or a part thereof as a consequence of reporter gene expression is indicative for the presence of undesirable herbivores in the soil. This would allow to use soil-targeted pesticides very purposefully and in amounts adequate to the pest infestation.

In a preferred embodiment, the present invention relates to recombinant nucleic acid molecules, wherein said heterologous coding sequence encodes an agent active against a herbivore.

In this context, an “agent active against a herbivore” is a polypeptide which, when expressed and brought in contact with a herbivore, especially by uptake of plant material into the digestive tract of the herbivore, reduces the damaging effect of the herbivore on the plant. For example, this agent may be a toxin such as the Bacillus thuringiensis (Bt) toxin, protease inhibitors, natural plant compounds with insect toxicity such as terpenes, phenolics and alkaloids and corresponding enzyme(s) catalyzing the production of such a compound.

According to this embodiment, it is of particular advantage that, due to the expression characteristic of the regulatory sequence of the invention, the expression of the anti-herbivore agent can be limited to the time period when it is required, namely upon herbivore attack. This may meet the interests of a minimization of the presence of agents in crop plants which could potentially present a risk to human health or where unnecessary expression of the agent is undesirable, e.g., for energetic reasons.

In a further preferred embodiment, the heterologous coding sequence comprised in the recombinant nucleic acid molecule of the invention encodes a caryophyllene synthase, preferably from a dicotyledonous plant.

This embodiment could be interesting for applications where, according to the discovery underlying the present invention, resistance against root-damaging insects is aimed at by transforming a target plant with caryophyllene synthase, and where transformation with an endogenous caryophyllene synthase already existing in the plant may cause a co-suppression effect. Accordingly, in the case of maize, it may for example be desirable to transform the plant with a caryophyllene synthase of dicotyledonous origin. In order to achieve an optimal expression profile, the coding sequence encoding a dicotyledonous caryophyllene synthase may then be operably linked to a regulatory sequence of the invention. Corresponding dicotyledonous caryophyllene synthase cDNAs are described in the prior art literature from Arabidopsis, Cucumis and Artemisia (see references mentioned above).

Moreover, the invention relates to vectors, in particular plasmids, cosmids, viruses, bacteriophages and other vectors commonly used in genetic engineering, which contain the above-described regulatory sequence or corresponding recombinant nucleic acid molecule of the invention. In a preferred embodiment of the invention, the vectors of the invention are suitable for the transformation of fungal cells, cells of microorganisms such as yeast or bacterial cells, animal cells or, in particular, plant cells. In a particularly preferred embodiment such vectors are suitable for stable transformation of plants.

In another preferred embodiment, the vectors further comprise a heterologous coding sequence operably linked to said regulatory sequence contained in the vectors. The above explanations concerning vectors comprising the caryophyllene synthase-encoding polynucleotide of the invention apply herein accordingly.

Additionally, the present invention relates to a method for producing genetically engineered host cells comprising introducing the above-described regulatory sequences and the corresponding recombinant nucleic acid molecules or vectors of the invention into a host cell.

Another embodiment of the invention relates to host cells, in particular prokaryotic or eukaryotic cells, genetically engineered with the above-mentioned regulatory sequences and the corresponding recombinant nucleic acid molecules or vectors of the invention or obtainable by the above-mentioned method for producing genetically engineered host cells, and to cells derived from such transformed cells and containing said regulatory sequence, recombinant nucleic acid molecule or vector of the invention. In a preferred embodiment the host cell is genetically modified in such a way that it contains the regulatory sequence stably integrated into the genome. Preferentially, the host cell of the invention is a bacterial, yeast, fungus, plant or animal cell.

The transformation of the host cell with a polynucleotide or vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc.

Furthermore, the invention relates to a method for producing a transgenic plant, plant cell or plant tissue comprising the introduction of at least one of the above-described regulatory sequences of the invention or corresponding recombinant nucleic acid molecules or vectors of the invention into the genome of a plant, plant cell or plant tissue.

Preferably, said method comprises (a) the introduction of at least one of said regulatory sequences, recombinant nucleic acid molecules or vectors into the genome of a plant cell and (b) regenerating the cell of (a) to a transgenic plant or transgenic plant tissue. Optionally, the method may further comprise step (c) producing progeny from the plants produced in step (b).

In one aspect, the invention accordingly refers to transgenic plant cells being genetically engineered with a regulatory sequence of the invention or with corresponding recombinant nucleic acid molecules or vector of the inventions or being obtainable by the aforementioned method for producing a plant cell.

In a further aspect, the invention relates to transgenic plants or plant tissue comprising plant cells which are genetically engineered with the regulatory sequence of the invention or which contain the corresponding recombinant nucleic acid molecule or the vector of the invention or to transgenic plants obtainable by the method mentioned above.

Preferably, the transgenic plant is a maize plant. The above explanations concerning the production of transgenic plant cells, plant tissues and plants, as far as being applicable, are referred to herein as well.

The invention also relates to propagation material of the transgenic plants of the invention being transformed with the regulatory sequence of the invention or corresponding recombinant nucleic acid molecules or vectors, said material comprising plant cells according to the invention. The term “propagation material” comprises those components or parts of the plant which are suitable to produce offspring vegetatively or generatively. Suitable means for vegetative propagation are for instance cuttings, callus cultures, rhizomes or tubers. Other propagation material includes for instance fruits, seeds, seedlings, protoplasts, cell cultures etc. The preferred propagation materials are tubers and seeds.

The invention also relates to harvestable parts of the plants of the invention such as, for instance, fruits, seeds, tubers, rootstocks, leaves or flowers.

In a further embodiment, the present invention relates to the use of the regulatory sequence of the invention or of a corresponding recombinant nucleic acid molecule, vector, host cell or transgenic plant of the invention for establishing or enhancing resistance against a herbivore, preferably a root-damaging insect, in plants.

This embodiment is to be seen in the light of the above-stated explanations regarding the usefulness of the regulatory sequence of the invention.

Preferably, said use refers to plants being maize plants and root-damaging insects being the corn rootworm.

In a further aspect, the present invention refers to a method for breeding a plant having a resistance against a herbivore, said method comprising crossing and/or selfing progenitor lines and selecting for desirable traits, wherein said method is characterized by a selection step for progenitor lines and/or offspring that is/are capable of expressing the polypeptide of the invention. Preferably, the herbivore is a root-damaging insect and the resistance is due to the capacity of attracting entomopathogenic nematodes by way of releasing (E)-beta-caryophyllene.

This embodiment makes use of the contribution of the present invention for marker-assisted breeding approaches. Accordingly, the method of this embodiment comprises conventional plant breeding steps involving the selection for desirable traits such as yield or robustness against biotic or abiotic stress and, additionally, the selection for progenitor lines and/or offspring that show(s) the molecular trait that it expresses the caryophyllene synthase polypeptide of the invention.

According to the breeding method of the invention, the steps of crossing and/or selfing progenitor lines and selecting for desirable traits is understood to include all sorts of conventional breeding techniques that a skilled person commonly applies in order to achieve plants with certain desirable agronomical traits, such as new plant varieties or cultivars, elite lines and, in particular, commercial plant varieties.

Accordingly, the steps of crossing, selfing and selecting may be carried out as is appropriate for the respective plant species. In addition to such steps, the breeding method of the invention may also include one or more further steps, including the application of techniques that are generally considered as being unconventional, such as interspecific crossing or the propagation of a progenitor or pedigree generation by way of non-sexual processes including, for instance, in vitro propagation using cell culture methods as known in the art. It is furthermore envisaged that the breeding method of the invention may also encompass the use of one or more transgenic lines for instance as progenitor lines. Said one or more transgenic lines may be transformed with another trait but the presence of expressible caryophyllene synthase as disclosed above in the context of the present invention.

However, it is preferred that the present method is carried out according to conventional breeding methods, i.e. without the use of genetic engineering. This meets the demand of the consumers for non-genetically engineered crops. Moreover, the present breeding method may also include the production of hybrid seed as is for example customary practice with crops like maize.

According to the present invention, the step of selecting for progenitor lines or offspring that produce the polypeptide of the invention may be carried out at each suitable stage of the breeding process. For instance, it may be used for screening for progenitor lines with which the breeding is started. Alternatively, it may also be used for selecting those plants of a segregating progeny which are capable of expressing the polypeptide of the invention, in particular when it is known that germplasm introduced into the breeding process at a preceding stage does not contain the caryophyllene synthase trait, as is for example the case with many North American maize lines.

The selection for the capability to express the polypeptide of the invention may be carried out for example by directly determining the presence of the polypeptide of the invention using suitable techniques described in the prior art literature such as antibody-based techniques, e.g. ELISA. Since caryophyllene synthase expression may only occur upon induction, it may be necessary to provide suitable inducing conditions, e. g. by challenging the plant to be assayed with a not-damaging insect, prior to taking a sample for protein analyses. Such a challenge may be substituted by an equivalent treatment as for example mechanical wounding and/or contacting with an elicitor derived from the insect, if such a treatment is known to lead to induction in plants that contain an active caryophyllane synthase allele.

In the alternative, whether or not a plant involved in the breeding process expresses the polypeptide of the invention may likewise be indirectly determined by examining nucleic acid molecules encoding the polypeptide and/or being part of the gene encoding said polypeptide. Thus, the detection may preferably be targeted to the transcript encoding the polypeptide or the corresponding genomic sequences.

In a preferred embodiment of the breeding method of the invention, the molecular selection step comprises the amplification of the caryophyllene synthase-encoding polynucleotide of the invention or of the regulatory sequence of the invention or of a portion of said polynucleotide or regulatory sequence.

The term “amplification” encompasses any suitable technique known to a person skilled in the art by which a relatively small amount of a nucleic acid molecule or a portion thereof can be amplified in vitro to an amount which is susceptible to detection. Preferred amplification techniques include for example PCR, RT-PCR and NASBA.

The amplification product may then be detected by methods known in the art including ethidium bromide agarose gel electrophoresis, acryl amide gel electrophoresis, optionally involving detectable labeling of the product, capillary electrophoresis, DNA chip technology or suitable blot techniques or further techniques being based on the recognition of different conformations of the amplification product.

In a particularly preferred embodiment, said polynucleotide to be detected or portion thereof is RNA and a cDNA obtained from the RNA is amplified

Accordingly, the trait of being capable of expressing the polypeptide of the invention may be indirectly determined by detecting the mRNA encoding it. This is possible due to the result observed when making the present invention that transcriptional regulation is involved in the induction of caryophyllene synthase expression upon attack with a root-damaging insect (see appended Example 4 and FIG. 6). In view of this result, it is, however, also necessary to provide inducing conditions, e. g. by challenging the plant to be assayed with a root-damaging insect, prior to taking a sample for RNA analysis. Such a challenge may be substituted by an equivalent treatment as for example mechanical wounding and/or contacting with an elicitor derived from the insect, if such a treatment is known to lead to induction in plants containing an active caryophyllene synthase allele.

In a alternative preferred embodiment of the breeding method of the invention, said selection step comprises the screening for a mutation that imparts the expression of said polypeptide.

This mutation screening may be carried out according to genetic testing methods known to the person skilled in the art. Thereby, the nucleotide and amino acid sequences provided herein (SEQ ID NOs: 1 to 6) may be used as reference sequences for the wild-type state, i.e. for plants being capable of expressing the caryophyllene synthase polypeptide of the invention.

Corresponding mutations may be identified by comparing the sequence of a wild-type allele with that of a mutated allele not being capable of expressing a polypeptide of the invention.

A corresponding deleterious mutation may for example reside in the coding sequence (in particular non-sense or frameshift mutations, but also amino acid substitutions, e.g., at conserved positions essential for protein function), may affect the correct splicing of the primary transcript (e.g. by modifying an exon/intron junction) or may affect the correct transcription or translation of the gene (e.g. by deleterious changes in a regulatory sequence such as the promoter).

It is particularly preferred that, in the above-described breeding method of the invention, the plant is a maize plant, the root-damaging insect is the corn rootworm and the entomopathogenic nematode is Heterorhabditis megidis.

The present invention further relates to a plant obtainable by the above-described breeding method of the invention. This includes preferred forms of products of a breeding method such as seeds or other propagation material.

In accordance with the above explanations, the present invention refers in a further aspect to the use of the polynucleotides of the invention, of the polypeptide of the invention, of the binding molecule of the invention or of the regulatory sequence of the invention for selecting a plant having a resistance against a herbivore, preferably a root-damaging insect.

Preferably, the plant is maize and the root-damaging insect is the corn rootworm.

Likewise, the present invention relates to a method for selecting a plant having a resistance to a herbivore, preferably to a root-damaging insect, comprising selecting for a plant that is capable of expressing the polypeptide of the invention. Particularly preferred is selection for resistance against corn-rootworm in maize. The explanations given above, in particular in connection with the breeding method of the invention apply to this embodiment accordingly.

Yet another embodiment of the present invention relates to a kit comprising the above-described polynucleotide specifically hybridizing to a caryophyllene synthase-encoding polynucleotide of the invention or to a regulatory sequence of the invention or a binding molecule of the invention.

The kit may contain further components such a buffers or means for detecting the binding of the hybridizing polynucleotide or binding molecule to the target molecule. If the kit is intended for use in a nucleic acid amplification assay, the kit may contain as further components the materials necessary for carrying out the assay, i.e. suitable buffers and/or enzymes, and the hybridizing polynucleotides may be in the form of primer oligonucleotides, preferably at least one being detectably labeled. Further kits are conceivable in this context, such as kits containing the components for more sophisticated amplification techniques as for example the Taqman™ or the LightCycler™ technology.

The kit of the invention may advantageously be used for carrying out the breeding method of the invention or the corresponding use for selecting a plant having resistance against a herbivore, preferably a root-damaging insect. The parts of the kit of the invention can be packaged individually in vials or in combination in containers or multicontainer units. Manufacture of the kit preferably follows standard procedures which are known to the person skilled in the art.

Moreover, the present invention relates to the use of the transgenic plant of the invention or of propagation material thereof or of a plant obtainable by the breeding method of the invention or propagation material thereof together with entomopathogenic nematodes for growing said plant. Preferably, said transgenic plant or plant is maize, said propagation material is from a maize plant and such entomopathoganic nematode is Heterorhabditis megidis.

It is envisaged that the biological control accomplished by the attraction of entomopathogenic nematodes upon insect attack through the release of caryophyllene may be improved by adding the nematodes to the soil. The nematodes can be cultivated according to methods described in the prior art literature and known to the skilled person. Advantageously, the nematodes are deployed to the field when, or shortly before, attack by root-damaging insects can be expected. Alternatively, the nematodes can be deployed at a time relatively long before attack, for example together with sowing, since nematodes are able to persist for longer periods without nutrition or can feed on other insects present in the soil.

These and other embodiments are disclosed and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.google.de. An overview of patent information in biotechnology and a survey of relevant sources of patent information useful for retrospective searching and for current awareness is given in Berks, TIBTECH 12 (1994), 352-364.

Furthermore, the term “and/or” when occurring herein includes the meaning of “and”, “or” and “all or any other combination of the elements connected by said term”.

The present invention is further described by reference to the following non-limiting figures and examples.

The Figures show:

FIG. 1 shows a comparison of the deduced amino acid sequence of TPS23-Del with (E)-β-caryophyllene synthases from other plants (Arabidopsis thaliana AtTPS27, Cucumis sativus CsCS, Artemisia annua QHS1) and two sesquiterpene synthases from maize (TPS10-B73, TPS4-B73). Amino acids identical in all six proteins are marked by black boxes. Amino acids identical in at least four proteins or representing conservative changes are highlighted with gray boxes. The highly conserved DDxxD region is marked with a bar. The accession numbers are: AtTPS27 (AAO85539), CsCS (MU05952), QHS1 (AAL79181), TPS4-B73 (MS88571), TPS10-B73 (AAX99146).

FIG. 2 shows a dendrogram analysis of the (E)-β-caryophyllene synthase TPS23 and other plant terpene synthases. The analysis includes the amino acid sequences of the functionally related (E)-β-caryophyllene synthases AtTPS27 (Arabidopsis thaliana), CsCS (Cucumis sativus), QHS1 (Artemisia annua) and two sesquiterpene synthases from maize, TPS10-B73 and TPS4-B73, which are most closey related but have a different functionality. The dendrogram was constructed using a neighbor joining algorithm and a bootstrap sample of 100. The accession numbers of the sequences are: AtTPS27 (AAO85539), CsCS (AA05952), QHS1 (AAL79181), TPS4-B73 (AAS88571), TPS10-B73 (AAX99146).

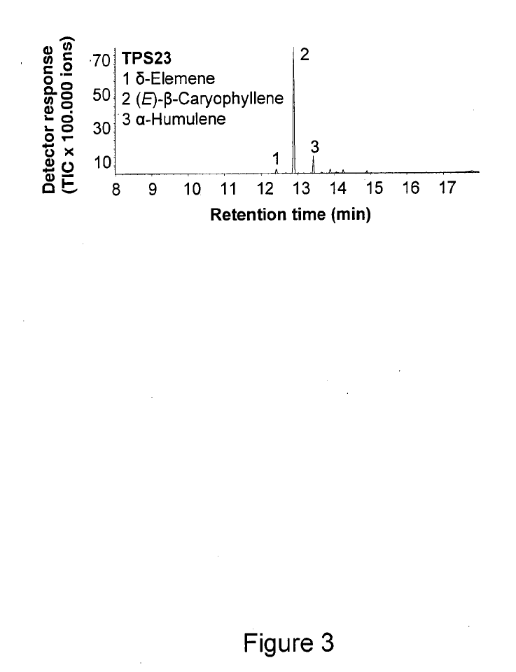

FIG. 3 shows an analysis of the sesquiterpene products of TPS23. The enzyme was heterologously expressed in E. coli, extracted, partially purified and incubated with the substrate (E,E)-FPP. The resulting terpene products were collected with a SPME fibre and analyzed by GC-MS. The products were identified as δ-Elemene (1), (E)-β-Caryophyllene (2) and α-Humulene (3) by comparison of their retention times and mass spectra to those of authentic standards.

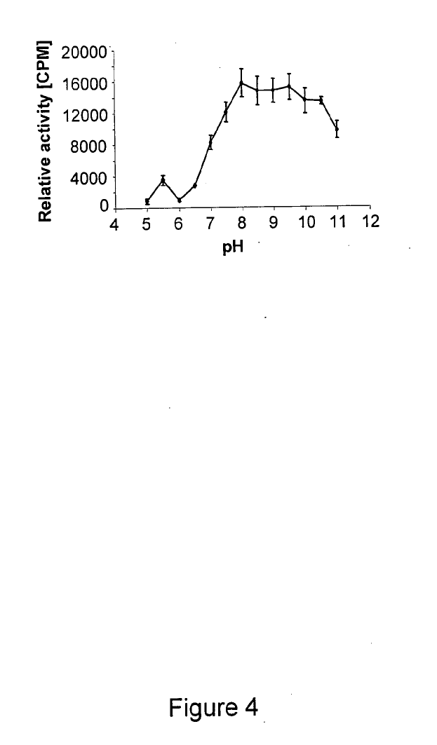

FIG. 4 shows that the enzymatic activity of TPS23 is pH dependent. The catalytic activity of the purified enzyme was measured in the presence of 10 mM Mg2+. Different pH values were adjusted with following buffers: pH 5.0 and 5.5: acetate buffer (100 mM); pH 6.0: MES buffer (100 mM); pH 6.5 to 9.5: bis-tris-propane buffer (100 mM); pH 10 to 11: CAPS buffer (10 mM). Means and SE of triplicate assays are shown.

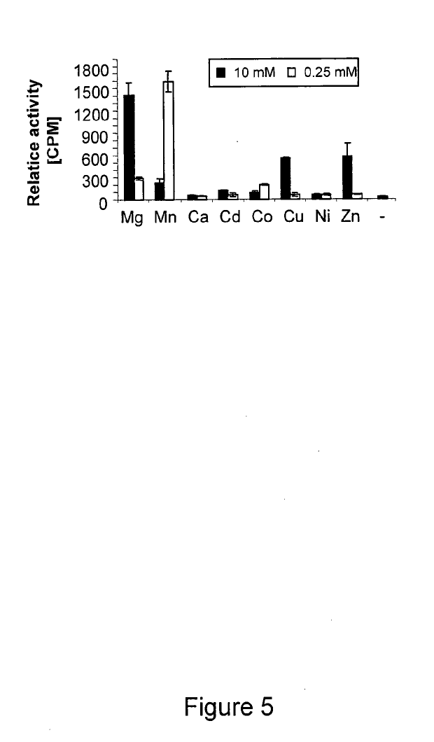

FIG. 5 shows that metal cofactors affect the enzymatic activity of TPS23. The catalytic activity of the purified enzyme was measured in the presence of various divalent metal ions at 10 or 0.25 mM. Means and SE of triplicate assays are shown.

FIG. 6 shows that the expression of TPS23 is strongly regulated by below- and above-ground herbivory. Transcript levels of tps23 in different organs of maize cultivars Delprim (A), Graf (B), B73 (C) and Pactol (D) after feeding of Spodoptera littoralis (Sp), Diabrotica virgifera virgifera (Dia), S. littorals and D. virgifera virgifera (Sp+Dia) or in undamaged controls (ctr). RNA isolated from 2 weeks old plants was blotted and hybridized with a probe specific for the first 400 bp of tps23. Blots were washed under high stringency conditions and exposed to a phosphor imager screen. The bottom panels show the 28s RNA band of the ethidium bromid stained RNA gels as a loading control.

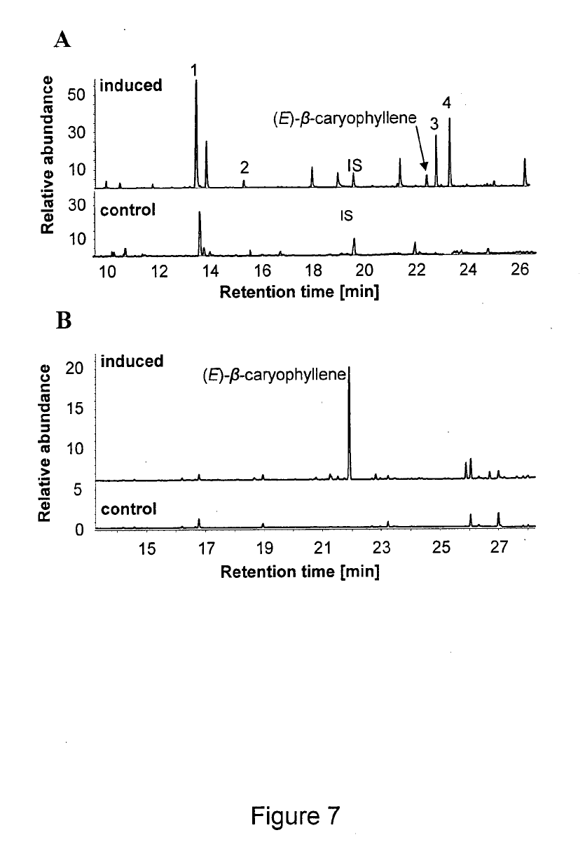

FIG. 7 shows that (E)-β-caryophyllene is emitted both in response to damage of the leaves by S. littoralis and attack of the roots by D. v. virgifera. (A) Volatiles from control leaves and leaves damaged by S. littoralis were collected and separated by gas chromatography. The major terpene compounds were identified as linalool (1), 4,8-dimethylnona-1,3,7-triene (2), (E)-α-bergamotene (3), and (E)-β-farnesene (4). Depicted are traces of the total ion current detector (B) Volatiles from control roots and leaves damaged D. v. virgifera. IS, internal standard—nonyl acetate.

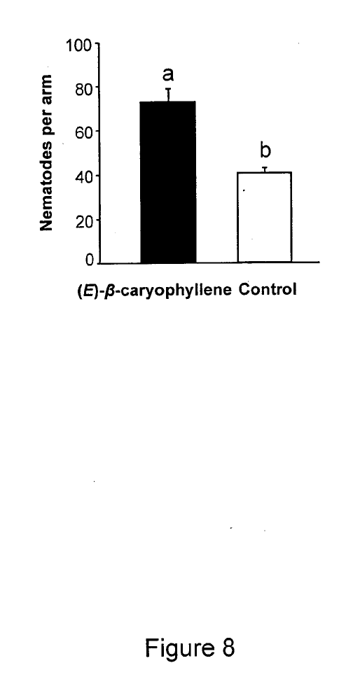

FIG. 8 shows that (E)-β-caryophyllene attracts nematodes. Attractiveness of (E)-β-caryophyllene to the entomopathogenic nematode Heterorhabditis megidis was demonstrated in six-arm olfactometers filled with moist sand. Of the nematodes that were released in the centers of the olfactometers, a significantly larger number was recovered from the arm connected to a pot spiked with (E)-β-caryophyllene, than from each of the five control arms (p<0.0001).

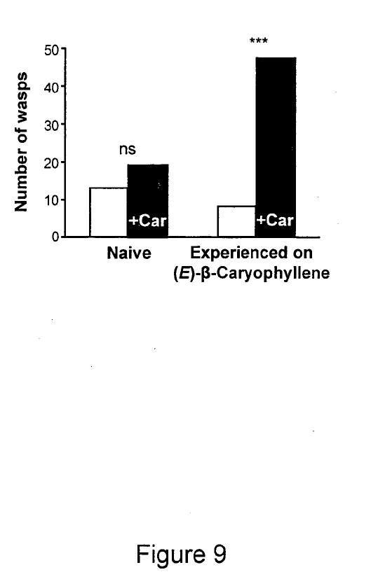

FIG. 9 provides evidence that the parasitic wasp Cotesia marginiventris is attracted by (E)-β-caryophyllene after previous oviposition experience. Responses of the parasitic wasp Cotesia marginiventris to (E)-β-caryophyllene: The attraction of parasitoid females to a pure standard of (E)-β-caryophyllene was tested in a four-arm olfactometer. Two groups of parasitoids were tested: naïve wasps and wasps with a previous oviposition experience on host larvae in the presence of (E)-β-caryophyllene. The parasitoids were tested in groups of six (n=14). The asterisks indicate a significant preference (p<0.0001) of experienced wasps for the odor of (E)-β-caryophyllene (solid bars) vs. pure air (empty bars shows average preference for one arm). Naïve wasps did not show this preference.

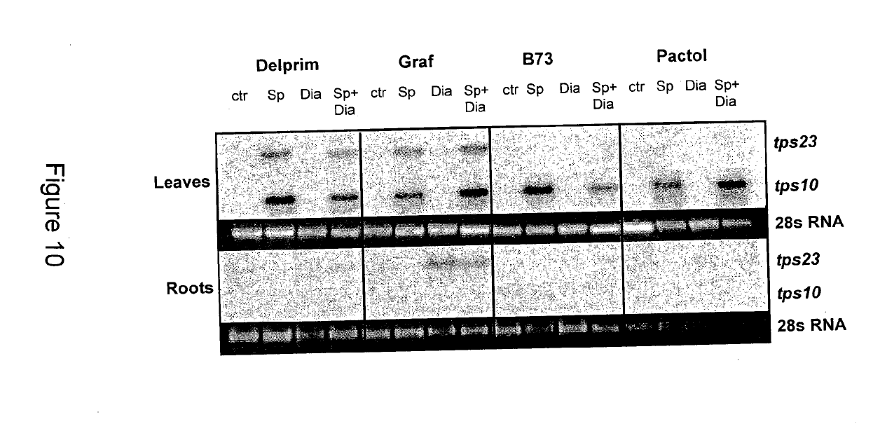

FIG. 10 essentially corresponds to FIG. 6 and additionally depicts expression of TPS10 upon herbivory of S. littoralis and D. v. virgifera. The transcript levels of tps23 and tps10 were determined in leaves and roots of the maize cultivars Delprim, Graf, B73 and Pactol after feeding of Spodoptera littoralis (Sp), 33 Diabrotica virgifera virgifera (Dia), and S. littorals plus D. v. virgifera (Sp+Dia) or in undamaged controls (ctr). RNA isolated from 2 week old plants was hybridized with probes specific for tps23 and tps10, respectively. The bottom panels show the 28s RNA band of the ethidium bromide stained RNA gels.

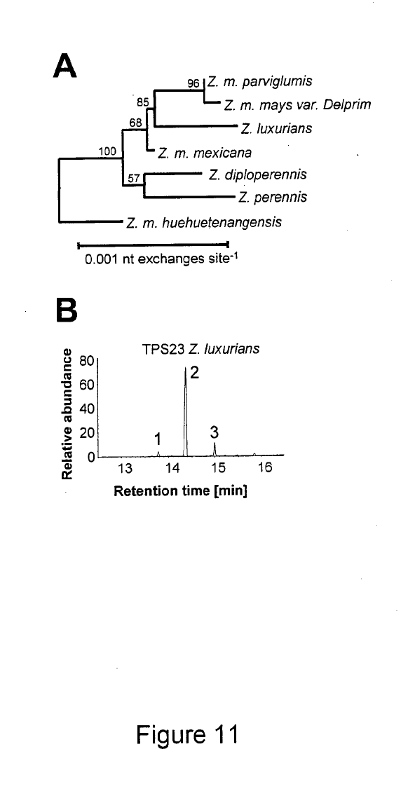

FIG. 11 shows that TPS23 is functionally conserved among relatives of maize. (A) Dendrogram analysis of TPS23 from maize (Zea mays mays) and its teosinte orthologs from Z. parviglumis, Z. luxurians, Z. mays mexicana, Z. diploperennis, Z. perennis, Z. huehuetenangensis. The analysis was conducted using a neighbor joining algorithm. Bootstrap values are shown in percent and generated with a sample of n=1000. (B) Sesquiterpene products of a TPS23 ortholog from Z. luxurians. The enzyme was expressed in E. coli, extracted, partially purified and incubated with the substrate (E,E)-FPP. The resulting terpene products were collected with a SPME fiber and analyzed by GC-MS. The products were identified as δ-elemene (1), (E)-β-caryophyllene (2) and α-humulene (3) by comparison of their retention times and mass spectra to those of authentic standards.

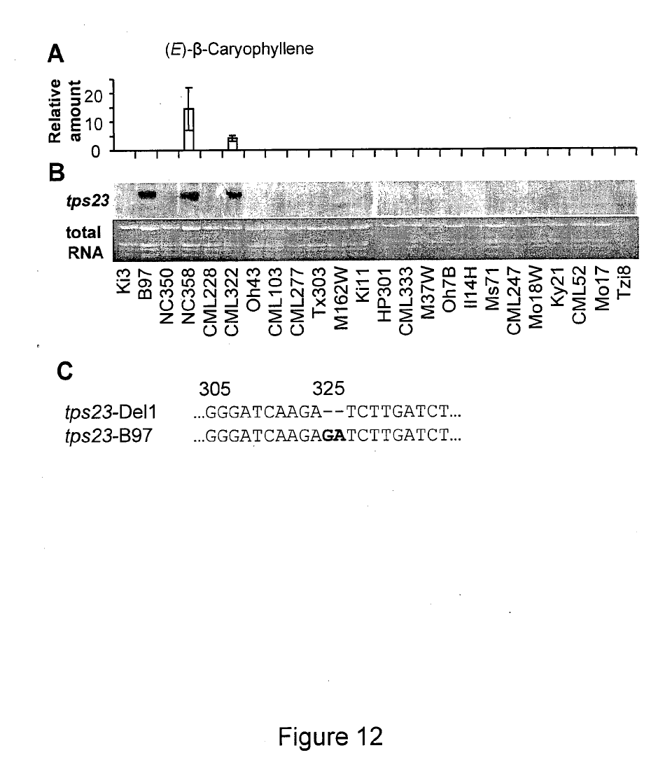

FIG. 12 shows that most maize lines of North American origin do not produce (E)-β-caryophyllene. A group of 24 inbred lines was tested for (E)-β-caryophyllene production (A) and accumulation of tps23 transcript (B) in herbivore-damaged leaves. The bottom panel of B shows the total RNA on the ethidium bromide stained gel as a loading control. (C) The tps23 allele of the inbred line B97 contains a 2 bp insertion at nt 315 compared to tps23-Del which results in an inactive enzyme.

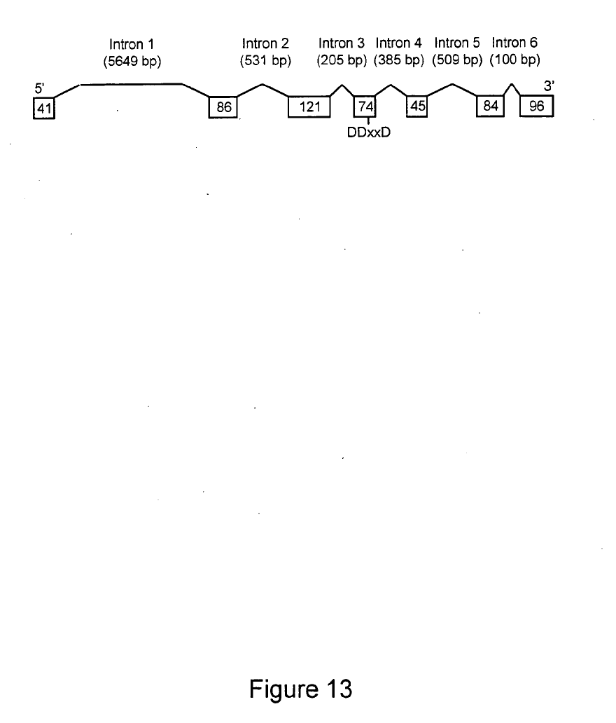

FIG. 13 depicts the exon-intron structure of tps23. The seven exons are represented by boxes showing the number of amino acids they contain. The first intron is enlarged by the insertion of a transposon-like sequence element of about 5.4 kb. DDxxD marks the position of the aspartate-rich region in the active center of the protein. The hatched line marks the transposon-like sequence included in the first intron.

The following Examples serve to further illustrate the invention.

In the Examples the following materials and methods were used.

1. Molecular Biological Techniques
Unless stated otherwise in the Examples, all recombinant DNA techniques are performed according to protocols as described in Sambrook and Russell (2001), Molecular Cloning: A Laboratory Manual, CSH Press, Cold Spring Harbor, N.Y., USA or in Volumes 1 and 2 of Ausubel et al. (1994), Current Protocols in Molecular Biology, Current Protocols. Standard materials and methods for plant molecular work are described in Plant Molecular Biology Labfase (1993) by R. D. D. Croy, jointly published by BIOS Scientific Publications Ltd (UK) and Blackwell Scientific Publications (UK).

2. Plant and Insect Material
Plants of the maize (Zea mays L.) varieties B73 (KWS seeds, Einbeck, Germany), Delprim (Delley Samen und Pflanzen, Delley, Switzerland), Graf (Landi) and Pactol (Syngenta) were provided by their respective breeders. The inbred lines F2, F476, Du101, W401, F7001 and F670 and seeds of the teosinte species were a gift from Station de Génétique Végétale, INRA, Gif-sur-Yvette, France, and the 24 North American inbred lines (small diversity panel) was supplied by the National Germplasm System of USDA-ARS, Beltsville, Md. The plants were grown in commercially available potting soil in a climate-controlled chamber with a 16 h photoperiod, 1 mmol (m²)⁻¹ s⁻¹ of photosynthetically-active radiation, a temperature cycle of 22° C./18° C. (day/night) and 65% relative humidity. Twelve to fifteen day old-plants (20-30 cm high, 4-5 expanded leaves) were used in all experiments. Eggs of Spodoptera littoralis Boisd. (Lepidoptera: Noctuidae) were obtained from Aventis (Frankfurt, Germany) and were reared on an artificial wheat germ diet (Heliothis mix, Stonefly Industries, Bryan, Tex., USA) for about 10-15 d at 22° C. under an illumination of 750 μmol (m²)⁻¹ s⁻¹. For the Spodoptera treatments, three third instar larvae were enclosed on the middle portion of each plant in a cage made out of two halves of a Petri dish (9 cm diameter) with a circle cut out of each side and covered with gauze to allow for ventilation (Rose et al. 1996). Larvae from Diabrotica virgifera virgifera LeConte were obtained from the University of Neuchatel, Neuchatel, Switzerland or from CABI BioSience (Delémont, Switzerland). Nematodes of the species Heterorhabditis megidis were supplied by Andermatt Biocontrol AG (Grossdietwil, Switzerland). For the D. v. virgifera treatment, each maize plant was subjected to four second instar or third instar larvae for two days.

3. cDNA Library Construction
Ten day-old maize plants of the cultivar Delprim were subjected to herbivory by Spodoptera littoralis for four hours. One gram of leaf material was ground in a mortar to a fine powder in liquid nitrogen and added to 10 ml of Trizol Reagent (GIBCO BRL, Rockville, USA). The mixture was treated with a Polytron (Kinematika AG, Switzerland) for one minute and incubated for 3 min on ice. Total RNA was isolated according to manufacturer's instructions. From about 80 μg of total RNA, the mRNA was isolated utilizing poly-T coated ferromagnetic beads (Dynal, Sweden). The mRNA was transcribed into cDNA while constructing a Marathon RACE library according to manufacturer's instructions (Clontech, Palo Alto, Calif., USA).

4. Isolation of a Maize Terpene Synthase cDNA
Sequences with high similarity to plant terpene synthases were identified in BLAST searches of the TIGR Maize Database (http://maize.tigr.org/). One of these fragments (AZM4_{—}53695; SEQ ID NO: 7) was cloned, sequenced and extended towards the 5′ end by the Marathon RACE procedure (Clontech, Palo Alto, Calif.) with a cDNA library from herbivore-induced leaves of the maize cultivar Delprim. The complete sequence, amplified with the primers BH3fwd (ATGGCAGCTGAT2AGGCAAGATCCG; SEQ ID NO: 13) and BH2rev (TTAGTCTATTAGATGCACATACMTG; SEQ ID NO: 14) from a cDNA and introduced into the sequencing vector pCR®4-TOPO® (Invitrogen, Carlsbad, USA), contains an open reading frame of 1644 bp.
The apparent orthologs of tps23 from the teosinte species were cloned using cDNA from herbivore-induced leaves of each of the teosinte species and the primers mentioned above. Sequence analysis was performed with the DNASTAR suite of programs (Lasergene, Madison, Wis.), nucleotide substitution rates were determined according to the method by Nei and Gojobori (1986), and dendrograms were created using the TREECON software package (Van de Peer and De Wachter, 1994) using a neighbor-joining algorithm.

5. Heterologous Expression of Terpene Synthases
For expression with a N-terminal 8×His tag the ORF of tps23 was amplified with the primers BH8fwd (ATTGCCATGGCGCAGCTGATGAGGCAAGATCC; SEQ ID NO: 24) and BH9rev (ATTAGMTTCTTAGTCTATTAGATGCACATAC; SEQ ID NO: 25) and cloned as a NcoI-EcoRI fragment into the expression vector pHIS8-3. The construct was introduced into the E. coli strain BL21 (DE3) and fully sequenced to avoid errors introduced by DNA amplification. Liquid cultures of the bacteria harboring the expression constructs were grown at 37° C. to an OD₆₀₀ of 0.6. Then, isopropyl-β-thiogalactopyranoside was added to a final concentration of 1 mM, and the cultures were incubated for 20 hours at 18° C. The cells were collected by centrifugation and disrupted by a 4×30 s treatment with a sonicator (Bandelin UW2070) in chilled extraction buffer (50 mM Mopso, pH 7.0, with 5 mM MgCl₂, 5 mM sodium ascorbate, 0.5 mM PMSF, 5 mM dithiothreitol and 10% (v/v) glycerol). The cell fragments were removed by centrifugation at 14,000 g and the supernatant was desalted into assay buffer (10 mM Mopso, pH 7.0, 1 mM dithiothreitol, 10% (v/v) glycerol) by passage through a Econopac 10DG column (BioRad, Hercules, Calif., USA). For kinetic studies the His-tagged enzyme was further purified on a nickel-nitrilotriacetate agarose column (Qiagen, Heidelberg, Germany) according to manufacturer's instructions.

6. Assay for Terpene Synthase Activity
To determine the catalytic activity of the terpene synthase TPS23, enzyme assays containing 50 μl of the bacterial extract and 50 μl assay buffer with 10 μM (E,E)-FPP, 10 mM MgCl₂, 0.05 mM MnCl₂, 0.2 mM NaWO₄ and 0.1 mM NaF in a Teflon-sealed, screw-capped 1 ml GC glass vial were performed. A SPME (solid phase microextraction) fiber consisting of 100 μm Polydimethylsiloxane (SUPELCO, Belafonte, Pa., USA) was placed into the headspace of the vial for 1 h incubation at 30° C. For analysis of the adsorbed reaction products, the SPME fiber was directly inserted into the injector of the gas chromatograph.
For the determination of metal ion cofactors, Kₘ values and effects of pH, an assay containing 1 μM purified TPS23 protein, 10 μM [1-³H](E,E)-farnesyl diphosphate (37 GBq mol⁻¹, American Radiolabeled Chemicals, St. Louis, Mo., USA) and 10 mM MgCl₂ in 100 μl assay buffer was used. The assay was overlaid with 1 ml pentane to trap volatile products and incubated for 20 min at 30° C. The reaction was stopped by mixing, and 0.5 ml of the pentane layer was taken for measurement of radioactivity by liquid scintillation counting in 2 ml Lipoluma cocktail (Packard Bioscience, Groningen, The Netherlands) using a Packard Tricarb 2300TR liquid scintillation counter (³H efficiency=61%). The pH optimum was determined in buffers from pH 5.0 to pH 11.0. Assay results are reported as the mean of three independent replicate assays, and each experiment was repeated 2-3 times with similar results. The Kₘ values were determined using seven substrate concentrations with four repetitions each. The enzyme activity was stable for at least 1 month when stored at −80° C. The concentration of the purified protein was determined by the method of Bradford (1976) using the BioRad reagent with BSA as standard.

7. Gas Chromatography
A Hewlett-Packard model 6890 gas chromatograph was employed with the carrier gas He at 1 ml min⁻¹, splitless injection (injector temperature—220° C.), a Chrompack CP-SIL-5 CB-MS column ((5%-phenyl)-methylpolysiloxane, 25 m×0.25 mm i.d.×0.25μ film thickness, Varian, USA) and a temperature program from 40° C. (3-min hold) at 5° C. min⁻¹ to 240° C. (3 min hold). The coupled mass spectrometer was a Hewlett-Packard model 5973 with a quadrupole mass selective detector, transfer line temperature—230° C., source temperature—230° C., quadrupole temperature—150° C., ionization potential—70 eV and a scan range of 40-350 atomic mass units. Products were identified by comparison of retention times and mass spectra with authentic reference compounds.

8. Northern Blotting
Plant RNA was prepared with the RNeasy plant mini kit (Qiagen, Hilden, Germany) according the manufacturer's instructions. A 400 bp fragment containing the first two exons of tps23 was used as a probe, generated by linear PCR with the primer 5′-GAACTTCAAAAATACATCAGA-3′ (SEQ ID NO: 10) and the complete ORF as a template. The probe was labeled with 32P-adenosine triphosphate using the Strip-EZ PCR procedure (Ambion, TX, USA). Blotting on a Nytran-Plus nylon membrane (Schleicher & Schuell, Germany), hybridization and washing were carried out following standard procedures. The blots were scanned with a Storm 840 Phosphoimager (Molecular Dynamics, Sunnyvale, Calif.).

9. Bioassays
Attraction of the nematode Heterorhabditis megidis toward (E)-β-caryophyllene was tested with belowground six-arm olfactometer assays (Rasmann et al., 2005). The apparatus consisted of a central glass chamber with six evenly distributed side arms that connect it to six glass pots. The entire system was filled with moist sand (10% water). An aliquot of 0.2 μl of authentic (E)-β-caryophyllene (98% pure; Sigma-Aldrich) was injected into one of the glass pots and the five untreated pots were used as controls. About 2000 H. megidis were released in a drop of water in the centre of the central arena. Ultra-fine screens at the end of each olfactometer arm prevented the nematodes from entering the pots. These arms consist of detachable parts from which nematodes can be recovered (for details see Rasmann et al., 2006). Our study used six belowground olfactometers simultaneously. Twenty-four hours after H. megidis release, the below ground olfactometers were disassembled and the sand from each arm was placed on separate cotton filter disks (Hoeschele GmbH) in Bearmann extractors (Curran, 1992; Hass et al., 1999). On the next day, recovered nematodes were counted.
To test the above ground role of (E)-β-caryophyllene in attracting herbivore enemies, S. littoralis caterpillars and the solitary endoparasitoid Cotesia marginiventris were reared as described by Turlings et al. (2004). In the tests, mated, 2-5 day-old females, 22 both naive and experienced individuals were used. Experienced females were obtained by placing them in a tube containing 20 S. littoralis larvae on top of a vessel that was connected via a glass capillary to a 2 ml glass vial filled with 300 μl of synthetic (E)-β-caryophyllene (Fluka, 99% purity). The release rate was calibrated to the range of (E)-β-caryophyllene concentrations that are released by maize plants. The wasps were released in the tube one at a time and removed after 3-5 ovipositions. For each replicate, 6 wasps were provided with this oviposition experience and subsequently the procedure was repeated with 20 fresh larvae and 6 new wasps.
The attractiveness of the (E)-β-caryophyllene to C. marginiventris females was tested in a 4-arm olfactometer as described by D'Alessandro and Turlings (2005). In all experiments, the (E)-β-caryophyllene-releasing device was installed in the airflow of one of the four olfactometer arms. The experiment was repeated seven times.
The behavioral responses of parasitoids and entomopathogenic nematodes to (E)-β-caryophyllene were analyzed with a log-linear model corrected for the expected distribution within the olfactometer (Turlings et al. 2004). The model was fitted by maximum quasilikelihood estimation in the software package R (Version 2.4.0) and its adequacy was assessed through likelihood ratio statistics and examination of residuals.

10. Volatile Collection
For the analysis of volatile terpenes, leaf material was frozen in liquid nitrogen and pulverized in a mortar. An aliquot of 0.2 g plant powder was placed in a glass vial with a septum in the lid. A 100 μm PDMS solid phase micro extraction (SPME) fiber (Supelco, 23 Belafonte, Pa., USA) was inserted through the septum and exposed for 60 min at 40° C. The compounds adsorbed onto the fiber were analyzed by GC-MS.

11. Isolation of the 5′- and 3′-Flanking Regions as Well as Intron 1 of tps23
For each of the maize lines assayed, genomic DNA was prepared with the DNeasy plant mini kit (Qiagen, Hilden, Germany) according the manufacturer's instructions. The Universal GenomeWalker™ Kit (Clontech, Palo Alto, Calif.) was used to isolate a 1.8 kb DNA fragment upstream of the tps23 open reading frame. To compare promoter alleles from different maize cultivars, the promoter fragments were amplified from genomic DNA by nested PCR using the primers BH13fwd (GTTAGTCCMTATTTGTGTTGGGC; SEQ ID NO: 15), BH12rev (GACGGATCTTGCCTCATCAGCTGCC; SEQ ID NO: 16) and BH14fwd (TTCMCCACCAAAATTAATACTGGG; SEQ ID NO: 17), BH11rev (GTATACTAGCTAGCTACTCTCCTGC; SEQ ID NO: 18), respectively, cloned into the sequencing vector pCR®4-TOPO® (Invitrogen, Carlsbad, USA) and fully sequenced. To analyze the downstream region of tps23, a 1.5 kb fragment containing 880 bp of the untranslated 3′ region was amplified with the primers BH31fwd (GTGCTATMTGCCGAGACAGAATGGCGTGACMG; SEQ ID NO: 19) and BH32rev (CAATTCATGTGGATTGGGTAGGATTGAGTGGGTTTC; SEQ ID NO: 20), cloned and sequenced as described above. To test for the presence of the unusually large intron 1, PCR was performed with the gene-specific primer BH3fwd located on exon 1 (see above) and the intron-specific primer BH26rev (GATCTMGGCCGTGTTTTATTCGC; SEQ ID NO: 21). The resulting 600 bp fragment was cloned and sequenced. The complete intron 1 was isolated from the inbred lines B73 and F2 using nested PCR with the primers BH3fwd, BH22rev (AGTAACATTTTCTTCACCTCCTCC; SEQ ID NO: 22) and BH27fwd (CACAGTGAGGAGGACATGCATGGG; SEQ ID NO: 23), BH21rev (ATTTCGACGTTATCCTTCATAATC; SEQ ID NO: 24), respectively.

### EXAMPLE 1

### Cloning of the Maize Terpene Synthase Gene tps23

A public maize genome database (http://maize.tigr.org/) was screened for sequences with similarity to known terpene synthases. One of the resulting fragments (AZM4_{—}53695; SEQ ID NO: 7) contains the last two exons and the last intron of a putative sesquiterpene synthase gene. The 5′ end of this fragment was extended by the Marathon RACE procedure (Clontech, Palo Alto, Calif.) with a cDNA library from herbivore-induced leaves of the maize cultivar Delprim to obtain the complete open reading frame (SEQ ID NO: 1). The cDNA contains an ORF of 1.644 bp designated as tps23-Del that encodes a protein (SEQ ID NO: 2) with a predicted molecular mass of 63.6 kDa. Numerous amino acids throughout its sequence are highly conserved among members of the terpene synthase family (FIG. 1). The most characteristic element is an Asp-rich DDxxD motif in the C-terminal part of the protein that is involved in the binding of the divalent metal cofactor (Starks et al., 1997). The gene only has a very low amino acid identity to other maize terpene synthases like TPS10 (40.5%, Schnee et al., 2006) and TPS4 (37.8%, Kollner et al., 2004). No genes with higher sequence identity were found after repeated PCR with maize cDNA and RACE libraries as well as in the maize genomic databases, suggesting that it is a single gene. Also, the caryophyllene synthases AtTPS27, CsCS, QHS1 exhibit a low amino acid identity of 32.9%, 30.3% and 35.1%, respectively. A dendrogram analysis demonstrates that TPS23 is more closely related to functionally unrelated terpene synthases of maize than to terpene synthases of similar function in other plant species, suggesting a convergent evolution of TPS23 (FIG. 2).

An allelic variant of the above-mentioned TPS23 sequence has also been isolated from maize cultivar Delprim. Its coding sequence and the deduced amino acid sequence is shown in SEQ ID NO: 3 and 4.

### EXAMPLE 2

### Heterologous Expression of TPS23 and Product Identification

Because it is still not possible to predict the product specificity of a putative terpene synthase from their amino acid sequence, the gene TPS23-Del was cloned with an N-terminal His-tag in the bacterial expression vector pHIS8-3. After expression in E. coli BL21 (DE3,) the recombinant protein was extracted, purified and incubated with the potential substratesgeranyl diphosphate (GPP, C10), farnesyl diphosphate (FPP, C15), and geranylgeranyl diphosphate (GGPP, C20). The enzyme did not accept GPP or GGPP as a substrate (data not shown), converting only FPP to terpene products (FIG. 3). The major product formed from FPP was identified as (E)-β-caryophyllene and the two minor products were α-humulene and δ-elemene, as determined by comparison of their retention times and mass spectra to those of authentic standards.

### EXAMPLE 3

### Biochemical Characterization of TPS23

To determine the biochemical properties of TPS23-Del, the purified enzyme was incubated with tritium-labeled FPP and the assays performed under the appropriate conditions. The enzyme exhibited a broad catalytic optimum from pH 8.0 to pH 9.5, but still shows substantial activity at typical cytoplasmic pH conditions (FIG. 4). The Kₘ value for FPP was 2.4±0.4 μM and the k_{cat} value was 0.0030±0.0002 s⁻¹. Both values are similar to those found for most terpene synthases that have been characterized in other plant species.

A divalent metal ion cofactor was required for enzyme activity (FIG. 5). Of the divalent cations tested, Mg²⁺ ions in a concentration of 10 mM and Mn²⁺ ions in a concentration of 0.25 mM gave substantial activities. The Kₘ values were 183±34 μM and 28±6 μM for Mg²⁺ and Mn²⁺, respectively (see Table 1).

TABLE 1
Kinetic constants for TPS23-Del heterologously expressed in E. coli.

| | FPP | FPP | | |
|---|---|---|---|---|
| | 10 mM MgCl₂ | 0.25 mM MnCl₂ | Mg²⁺ | Mn²⁺ |
| Kₘ | 3.7 ± 0.5 | 1.1 ± 0.3 | 183 ± 34 | 28 ± 5 |
| [μM] | | | | |
| k_{cat} | (1.91 ± 0.09) × 10⁻³ | (1.13 ± 0.07) × 10⁻³ | | |
| [s⁻¹] | | | | |

Both the Kₘ and the k_{cat} values for the farnesyl diphosphate substrate are similar to those of most characterized plant sesquiterpene synthases identified to date (Chen et al., 1996; Crock et al., 1997; Picaud et al., 2005; 2006).

The properties of maize TPS23 are similar to those of other plant caryophyllene synthases from Artemisia annua (Cai et al., 2002), Cucumis sativus (Mercke et al., 2004) and Arabidopsis thaliana (Tholl et al., 2005) in kinetic parameters and cofactor requirement. These enzymes also have a common reaction mechanism, as indicated by the formation of the same minor products, α-humulene and δ-elemene. The reaction is initiated by a metal cofactor-assisted dephosphorylation to form a carbocationic species. Then, electrophilic attack of C1 on the C10-C11 double bond forms a C1-C11 ring that is converted to caryophyllene by an additional C2-C10 cyclization and a loss of a proton (Cai et al., 2002; Mercke et al., 2004; Tholl et al., 2005). α-Humulene and δ-elemene are likely produced by premature proton loss or rearrangement prior to the second cyclization.

Despite these similarities, sequence comparisons indicate that the (E)-β-caryophyllene synthases are products of convergent evolution, as TPS23 is more closely related to other maize terpene synthases than to the other (E)-β-caryophyllene synthases isolated from dicotyledons. One such closely related maize terpene synthase is TPS10, which produces a complex blend of volatile sesquiterpenes, including (E)-β-farnesene and (E)-α-bergamotene, upon herbivore damage that attracts parasitic wasps (Schnee et al., 2006). It is conceivable that TPS23 and TPS10 are derived from a common precursor involved in indirect defense and that the mechanism to produce (E)-β-caryophyllene has been acquired subsequently by TPS23. Such convergent evolution is probably facilitated by the ability of terpene synthases to alter product specificity in response to only a few amino acid changes (Köllner et al., 2004b; Köllner et al., 2006; Yoshikuni et al., 2006).

Small amounts of (E)-β-caryophyllene are also formed as part of a complex sesquiterpene blend produced by all parts of the maize plant (Köllner et al., 2004a). This blend, which has been designated group B, is not affected by herbivory of Spodoptera littoralis (Köllner et al., 2004a), indicating that (E)-β-caryophyllene is also formed as minor product of another maize terpene synthase.

### EXAMPLE 4

### Transcript Levels of TPS23 are Induced by Herbivory

In recent publications it was shown that some maize varieties, mostly from the North American maize breeding programs, are not able to produce (E)-β-3-caryophyllene after attack of leaf herbivores (Degen et al., 2004) or root herbivores (Rasmann et al., 2005). To analyze whether TPS23 is involved in the synthesis of (E)-β-caryophyllene after herbivory, the transcript level of tps23 was measured in leaves and roots of two (E)-β-caryophyllene-producing and two non-producing maize varieties after attack with larvae of Spodoptera littoralis and Diabrotica virgifera virgifera. A probe specific for the first two exons of tps23 was used to detect the level of tps23 transcript in RNA-blot experiments. Transcripts were detected only in the two European, (E)-β-caryophyllene-producing cultivars Delprim and Graf (FIG. 6). Diabrotica feeding on the roots resulted in transcript accumulation in the roots while herbivory by Spodoptera on the leaves led to the accumulation of tps23 transcripts in the leaves. The higher transcript levels in the roots of the variety Graf compared to the variety Delprim correlate with the production of more (E)-β-caryophyllene in this line (Rasmann et al., 2005).

The inbred line B73 and the cultivar Pactol are the result of North American breeding programs and do not form detectable levels of tps23 transcripts. After feeding of larvae, transcript levels of tps23 are induced in leaves The combined feeding of D. v. virgifera on the roots and S. littoralis on the leaves does not influence the transcript level of tps23 in leaves but appears to reduce transcript levels in the roots.

The tps23 expression pattern was compared with that of tps10 under the same conditions. As expected, the transcript of the leaf-specific terpene synthase tps10 accumulated only in response to the aboveground damage by S. littoralis, but was not induced by belowground attack by D. v. virgifera (FIG. 10). Transcripts of tps10 are also present in caryophyllene and non-caryophyllene producing plants alike, consistent with earlier reports on the emission of TPS10 volatiles by lines B73 and Pactol (Gouinguené et al, 2001; Köllner et al., 2004a).

The expression pattern observed for tps23 deviates from those that are typically seen for defense-related genes. Enzyme activities in plants are often controlled by differential regulation of members of a gene family. In terpene biosynthesis, the enzymes of important regulatory steps, such as 3-hydroxy-3-methylglutaryl-CoA synthase (Enjuto et al., 1995; Daraselia et al., 1996; Korth et al., 1997), 1-deoxy-xylulose phosphate synthase (Walter et al., 2002) and isoprenyl diphosphate synthases (Cunillera et al., 1997; Okada et al., 2000) are encoded by small gene families with differential expression. However, tps23 provides an example of a single gene with two distinct expression patterns in different organs. The above ground induction of tps23 is similar to that of tps10, a maize terpene synthase gene that is activated after herbivory by lepidopteran larvae on leaves and produces most of the herbivore-induced sesquiterpene hydrocarbon volatiles of maize (Schnee et al., 2006). In contrast, only tps23, and not tps10, is activated below ground. It is conceivable that tps23 and tps10 genes share the same regulatory mechanism for above ground induction, but that tps23 contains an additional promoter element that activates the gene in the root after herbivory.

### EXAMPLE 5

### Isolation of TPS23 Promoter

The promoter sequences of the TPS23 gene were isolated from genomic DNA prepared from plants of the maize variety Delprim. For isolation of the promoter sequence, the “Genome Walker Kit” of Clontech, Palo Alto, Calif., USA was used according to manufacturer's instructions. Briefly, the genomic DNA is cut into fragments by several restriction nucleases and the fragments are ligated to a double stranded adaptor fragment of known sequence. The promoter was isolated by a nested PCR protocol using an adaptor-specific primer and the primer BH12 GACGGATCTTGCCTCATCAGCTGCC (SEQ ID NO: 11) as outer, caryophyllene synthase-specific primer in the first PCR reaction and the primer BH11 GTATACTAGCTAGCTACTCTCCTGC (SEQ ID NO: 12) as inner primer for the second PCR. The procedure was repeated three times to ensure that the promoter sequence is free of PCR artifacts.

The resulting promoter sequences Del-A and Del-B are shown in SEQ ID NOs: 5 and 6, respectively. Note that the depicted DNA sequences contain as the last three residues the start codon ATG of the TPS23 reading frame.

### EXAMPLE 6

### Caryophyllene is Attractive to Two Types of Herbivore Enemies, Entomopathogenic Nematodes and Parasitic Wasps

Corn plants damaged by lepidopteran larvae release a complex blend of volatiles that is dominated by a large number of sesquiterpene olefins. In the attempt to identify the compound(s) that are responsible for interactions with other organisms, (E)-β-farnesene and (E)-α-bergamotene were previously identified as major constituents of a blend used by parasitic wasps to find their lepidopteran hosts (Schnee et al., 2006). Many maize lines, especially those originating from European breeding programs, also emit the sesquiterpene (E)-β-caryophyllene after damage by lepidopterans (FIG. 7). In roots, (E)-β-caryophyllene is the sole compound released in significant amounts after damage by the herbivore D. v. virgifera. Experiments with a six-arm “below ground” olfactometer (filled with moist sand) confirmed that (E)-β-caryophyllene attracts the entomopathogenic nematode Heterorhabditis megidis Poinar (FIG. 8). Approximately twice as many nematodes were recovered on average from the arm of the olfactometer spiked with authentic caryophyllene as compared to the average for the five remaining arms that were not spiked (F1,34=13.13, P<0.0001) (FIG. 9).

To test for a possible above ground role of (E)-β-caryophyllene in attracting the parasitic wasp C. marginiventris, the pure compound was used for bioassays in a conventional four-arm olfactometer. Naïve wasps without any oviposition experience were not attracted (F1.82=0.98, P=0.32). However, wasps preferred air carrying (E)-β-caryophyllene to pure air (F1.82=52.06, P<0.0001) after they had experienced laying eggs in host larvae while perceiving (E)-β-caryophyllene (FIG. 3). This effect of associative learning was reflected in a significant treatment effect (F1.166=34.04, P<0.0001) and a significant treatment-experience interaction (F1.164=13.49, P<0.001).

The above-described results demonstrate that the sesquiterpene olefin, (E)-β-caryophyllene, can play a role in two spatially separate modes of induced defenses against herbivores: the attraction of parasitic wasps above ground that oviposit on lepidopteran larvae, such as S. littoralis, and the attraction of nematodes below ground that can attack larvae of the beetle D. v. virgifera. Both volatile signals are produced by a single enzyme, the terpene synthase TPS23. The attraction of the nematode to (E)-β-caryophyllene is innate (Rasmann et al., 2005), whereas females of the parasitic wasp are attracted only after associative learning (FIG. 9). Since (E)-β-caryophyllene is a constituent of many plant volatile blends, it can provide a reliable cue for generalist parasitoids that can find their herbivorous victims on many different plant species.

(E)-β-Caryophyllene is a volatile compound released by many plant species and so may serve as a general indirect defense signal, useful for generalist parasitoids or predators that attack a variety of different hosts or prey on different plant species. This compound diffuses rapidly in the air or soil (Hiltpold and Turlings, unpublished results). In addition, compared to most other volatile monoterpenes and sesquiterpenes, (E)-β-caryophyllene is very unstable in the atmosphere reacting readily with ozone and other reactive oxygen species (Grosjean et al., 1993). Thus (E)-β-caryophyllene may be diagnostic as a short-range cue for host or prey location. (E)-β-caryophyllene also has anti-microbial activity and so may function in direct defense against pathogens (Sabulal et al., 2006). Release of (E)-β-caryophyllene might have been initially selected for by pathogen attack and its signaling function in indirect defense evolved secondarily.

### EXAMPLE 7

### Maize tps23 and its Teosinte Orthologs are Maintained by Positive Selection

The appearance of (E)-β-caryophyllene in herbivore-induced volatiles of many grasses related to maize suggests that this compound has a widespread role in direct defense (Degen et al., 2004; Gouinguené et al., 2001). To learn more about the evolution of (E)-β-caryophyllene formation, the apparent orthologs of tps23 were isolated from six teosinte taxa utilizing PCR (FIG. 11). After expression in a bacterial system, all tps23 orthologs produced the (E)-β-caryophyllene main product as well as the characteristic by-products α-humulene and δ-elemene (FIG. 11B), demonstrating complete functional conservation of tps23 among maize and its close relatives. A dendrogram analysis of tps23 orthologs follows the phylogeny generally observed among the teosinte species (Buckler et al., 2006) and shows close nucleotide similarity among the sequences. A positive selection pressure for maintenance of (E)-β-caryophyllene synthase function is evident from the high average number of synonymous nucleotide changes relative to non-synonymous changes among tps23 from maize and its teosinte orthologs (dS/dN=6.88).

### EXAMPLE 8

### Lack of tps23 Transcript Prevents (E)-β-Caryophyllene Formation in Most North American Maize Lines

Initial studies by Degen et al, 2004 and Rasmann et al., 2005 suggested that maize lines originating from North American breeding programs have largely lost the ability to produce the (E)-β-caryophyllene signal. To determine the extent to which this defense trait was lost during domestication, (E)-β-caryophyllene production was studied in a set of 24 inbred ‘founder’ lines assembled to reflect a large percentage of the polymorphisms in North American maize (Liu et al., 2003). Of these 24 lines, only two, NC358 and CML322, were found to produce (E)-β-caryophyllene, suggesting that this trait is indeed largely absent from North American breeding lines (FIG. 12 A). The two (E)-β-caryophyllene-producing lines displayed transcripts of tps23 while no transcripts were observed in all other lines except one (FIG. 12B). This exceptional line, B97, accumulated tps23 transcripts although it does not produce (E)-β-caryophyllene. Sequencing of the tps23-B97 allele, showed a 2 bp insertion at position 315 which results in a frame shift that prevents the correct translation of the protein and thereby blocks (E)-β-caryophyllene production (FIG. 12C).

To understand how domestication and breeding may have caused the loss of this defense signal, the tps23 alleles of six (E)-β-caryophyllene producing lines (the hybrids Delprim, Graf and the inbred lines F2, F476, Du10 and W401) were compared with four non-producing lines (the hybrid Pactol and inbred lines B73, F7001 and F670). All lines contained at least one identical tps23 allele, indicating that the lack of transcript in some of the lines is not due to the presence of inactive alleles (Table 2).

TABLE 2
Properties of the tps23 alleles and their promoters in the hybrid lines
Delprim, Graf, and Pactol and the inbred lines B73, F2, F467, Du101,
W401, F670, F7001

| | | | Pro- | | | |
|---|---|---|---|---|---|---|
| | Caryo- | Intron | moter | Promoter | ORF | 3′ UTR |
| | phyllene | 5.5 kb | allele 1 | allele 2 | allele 1 | allele 1 |
| Delprim | + | + | + | + | + | + |
| Graf | + | + | − | + | + | + |
| F2 | + | + | + | − | + | + |
| F476 | + | + | + | − | not tested | + |
| Du101 | + | + | + | − | not tested | + |
| W401 | + | + | + | − | not tested | + |
| F670 | − | + | + | − | not tested | + |
| F7001 | − | + | + | − | not tested | + |
| B73 | − | + | + | − | + | + |
| Pactol | − | + | + | − | + | + |

Next, the genomic structure of tps23 was determined by analysis of the alleles tps23-B73 and tps23-F2. The structure of both alleles consists of seven exons and is generally similar to that of other terpene synthases from maize (Köllner et al., 2004b; Shen et al., 2001) and class III terpene synthases from other plants (Trapp and Croteau, 2001). Unlike other terpene synthases, however, the first intron is very large and contains transposon sequences, indicating that this intron was enlarged by transposon insertion from a size of about 121 bp, usually observed in terpene synthases, to 5.6 kb (FIG. 13). Since this insertion is observed in the tps23 alleles of all lines, regardless of (E)-β-caryophyllene production, it too is not likely to be responsible for inactivation of the gene in the non-producing lines. Further, a 1.8 kb promoter fragment was tested for specific differences that might regulate transcriptional activity of tps23 in the different maize lines. Two types of promoter sequence were found which are distinguished by 18 single base-pair changes throughout the fragment. A single base-pair change that created an EcoRI restriction site was located 425 bp in front of the transcription start. Among the hybrid lines, Pactol has only promoter type 1, Graf has only type 2, and Delprim has both. However, since all inbred lines had type 1 promoters regardless of their ability to produce (E)-β-caryophyllene, the changes in the 1.8 kb fragment did not account for differences in transcriptional activity of tps23. Similarly, the 880 bp 3′ untranslated region of all alleles was shown to be identical and can therefore not cause differences in transcriptional activity or changes in mRNA stability.

The experimental results taken together herein indicate that (E)-β-caryophyllene emission was lost by transcriptional inactivation during breeding of North American maize lines. (E)-β-caryophyllene is emitted from all tested maize lines from European breeding programs and from species of teosinte, the closest wild relative of maize. On the other hand, a range of inbred lines that represent approx. 85% of the genetic diversity of North American maize lines showed (E)-β-caryophyllene production in less than 10% of the lines. It is therefore assumed that this defensive trait was lost during breeding of North American maize lines. All lines that did not produce (E)-β-caryophyllene lack the tps23 transcript (with one explainable exception), indicating that the (E)-β-caryophyllene polymorphism results from differences in transcription. Differences in transcript stability can be ruled out, since the hypothetical tps23 transcript is identical in all maize lines analyzed regardless of (E)-β-caryophyllene production. The lack of transcription in most North American lines might be due to inactivation of a transcription factor or corruption of an enhancer element outside of the assayed promoter region. Whatever the identity of this factor or enhancer element, it is clearly not necessary for activation of tps10, which has a very similar expression profile to that of tps23 in leaves after S. littoralis attack.

The loss of defensive traits during crop domestication has frequently been postulated, but the genetic basis of this process, such as the inactivation of tps23 described here, has rarely been elucidated (Sotelo, 1997). The loss of tps23 expression might be ascribed to several causes. First, a null allele of a required transcription factor may be closely linked to a trait which is known to differ between North American and European maize lines, like flowering time. Breeding efforts to alter this trait could then have resulted in the accumulation of the null allele for (E)-β-caryophyllene production. Alternatively, the release of (E)-β-caryophyllene might be disadvantageous under conditions specific to North American agriculture and therefore have been selected against. A possible disadvantage of (E)-β-caryophyllene release in this scenario could be its reported attractiveness to adult females of D. v. virgifera (Hammack et al., 2001).

The use of natural enemies, such as entomopathogenic nematodes, is an important component of many integrated pest control programs and could reduce damage by D. v. virgifera, an economically important maize pest that causes extensive yield losses. The failure of past efforts to control this pest with nematodes in North America (Ellsbury et al., 1996; Jackson, 1996) may be due to the lack of (E)-β-caryophyllene release from maize lines under cultivation. (E)-β-caryophyllene release is correlated with increased nematode attraction to maize in the field (Rasmann et al., 2005). The identification of tps23 provides a molecular tool to devise alternate strategies for D. v. virgifera control. The restoration of (E)-β-caryophyllene production in non-producing maize lines should enhance their attractiveness to nematodes and thus increase D. v. virgifera mortality. Another strategy is the use of the tps23 promoter to control the expression of toxins, such as the Bacillus thuringensis Cry3 Bb 1 protein, that are effective against D. v. virgifera. This could provide the plant with an efficient, timely and well-localized defense against this pest.

### CITED LITERATURE

Bradford, M. M. (1976) A rapid and sensitive method for the quantitation of microgram quantities of protein utilizing the principle of protein-dye binding. Anal. Biochem. 72, 248-254.

Buckler, E. S., Goodman, M. M., Holtsford, T. P., Doebley, J. F., and Sanchez, J. (2006), Phylogeography of the wild subspecies of Zea mays. Maydica 51, 123-134

Cai, Y., Jia, J. W., Crock, J., Lin, Z. X., Chen, X. Y., and Croteau, R. (2002) A cDNA clone for β-caryophyllene synthase from Artemisia annua. Phytochemistry 61, 23-529.

Chen, X. J., Wang, M., Chen, Y., Davisson, V. J., and Heinstein, P. (1996) Cloning and heterologous expression of a second (+)-δ-cadinene synthase from Gossypium arboreum. J. Nat. Prod. 59, 944-951

Crock, J., Wildung, M., and Croteau, R. (1997) Isolation and bacterial expression of a sesquiterpene synthase cDNA clone from peppermint (Mentha×piperita, L.) that produces the aphid alarm pheromone (E)-β-farnesene. Proc. Natl. Acad. Sci. USA 94, 12833-12838

Curran, J. (1992). Influence of application method and pest population size on the field efficacy of entomopathogenic nematodes. Journal of Nematology 24, 631-6

Cunillera, N., Boronat, A., and Ferrer, A. (1997) The Arabidopsis thaliana fps1 gene generates a novel mRNA that encodes a mitochondrial farnesyl-diphosphate synthase isoform. Journal of Biological Chemistry. 272, 15381-15388

D'Alessandro, M., and Turlings, T. C. J. (2005) In situ modification of herbivore induced plant odors: A novel approach to study the attractiveness of volatile organic compounds to parasitic wasps. Chemical Senses 30, 739-753

Daraselia, N. D., Tarchevskaya, S., and Narita, J. O. (1996) The promoter for tomato 3-hydroxy-3-methylglutaryl coenzyme A reductase gene 2 has unusual regulatory elements that direct high-level expression. Plant Physiology 11, 727-733

Degen T. Dillmann C. Marion-Poll F. Turlings T C J (2004) High genetic variability of herbivore-induced volatile emission within a broad range of maize inbred lines. Plant Physiology. 135(4): 1928-1938

Ellsbury, M. M., Jackson, J. J., Woodson, W. D., Beck, D. L. and Stange, K. A. (1996) Efficacy, application distribution, and concentration by stemflow of Steinernema carpocapsae (Rhabditida: Steinernematidae) suspensions applied with a later move irrigation system for corn rootworm (Coleoptera: Chrysomelidae) control in maize. Journal of Economic Entomology 89, 74-81

Enjuto, M., Lumbreras, V., Marín, C., and Boronat, A. (1995) Expression of the Arabidopsis HMG2 gene, encoding 3-hydroxy-3-methylglutaryl coenzyme A reductase, restricted to meristematic and floral tissues. The Plant Cell 7, 517-527

Gouinguené, S., Degen, T., and Turlings, T. C. J. (2001) Variability in herbivore induced odour emissions among maize cultivars and their wild ancestors (teosinte). Chemoecology 11, 9-16

Grosjean, D., Williams, E. L., Grosjean, E., Andino, J. M. and Seinfeld, J. H. (1993) Atmospheric oxidation of biogenic hydrocarbons: Reaction of ozone with β-pinene, D-limonene and trans-caryophyllene. Environmental Science and Technology 27, 2754-2758

Hammack, L. (2001) Single and blended maize volatiles as attractants for diabroticite corn rootworm beetles. Journal of Chemical Ecology 27, 1373-1390

Hass, B., Griffin, C. T. and Downes, M. J. (1999) Persistence of Heterorhabditis infective juveniles in soil: Comparison of extraction and infectivity measurements. Journal of Nematology 31, 508-516

Jackson, J. J. (1996) Field performance of entomopathogenic nematodes for suppression of western corn rootworm (Coleoptera: Chrysomelidae). Journal of Economic Entomology 89, 366-372

Köllner, T. G., Schnee, C., Gershenzon, J., and Degenhardt, J. (2004a) The sesquiterpene hydrocarbons of maize (Zea mays) form five groups with distinct developmental and organ-specific distributions. Phytochemistry 65, 1895-1902

Köllner T G, Schnee C, Gershenzon J, Degenhardt J (2004b) The variability of sesquiterpenes emitted from two Zea mays cultivars is controlled by allelic variation of two terpene synthase genes encoding stereoselective multiple product enzymes. Plant Cell. 16: 1115-1131

Köllner, T. G., O'Maille, P. E., Gatto, N., Boland, W., Gershenzon, J. and Degenhardt, J. (2006) Two pockets in the active site of maize sesquiterpene synthase TPS4 carry out sequential parts of the reaction scheme resulting in multiple products. Archives of Biochemistry and Biophysics, 448, 83-92

Korth, K. L., Stermer, B. A., Bhattacharyya, M. K., and Dixon, R. A. (1997) HMGCoA reductase gene families that differentially accumulate transcripts in potato tubers are developmentally expressed in floral tissues. Plant Molecular Biology. 33, 545-551

Liu, K., Goodman, M., Muse, S., Smith, J. S., Buckler, E., and Doebley, J. (2003) Genetic structure and diversity among maize inbred lines as inferred from DNA microsatellites. Genetics 164: 2117-28

Mercke, P., Kappers, I. F., Verstappen, F. W. A., Vorst, O., Dicke, M., and Bouwmeester, H. J. (2004) Combined transcript and metabolite analysis reveals genes involved in spider mite induced volatile formation in cucumber plants. Plant Physiology 135, 2012-2024

Nei, M., and Gojobori, T. (1986) Simple methods for estimating the numbers of synonymous and nonsynonymous nucleotide substitutions. Molecular Biology and Evolution 3, 418-26.

Okada, K., Saito, T., Nakagawa, T., Kawamukai, M., and Komiya, Y. (2000) Five geranylgeranyl diphosphate synthases expressed in different organs are localized into three subcellular compartments in Arabidopsis. Plant. Physiol. 122, 1045-1050

Picaud, S., Brodelius, M., and Brodelius, P. E. (2005) Expression, purification and characterization of recombinant (E)-β-farnesene synthase from Artemisia annua. Phytochemistry 66, 961-967

Picaud, S., Olsson, M. E., Brodelius, M., and Brodelius, P. E. (2006) Cloning, expression, purification and characterization of recombinant (+)-germacrene D synthase from Zingiber officinale. Arch. Biochem. Biophys. 452, 17-28

Rasman S, Köllner T G, Degenhardt J, Hiltpold I, Töpfer S, Kuhlmann U, Gershenzon J, Turlings T C J (2005) Recruitment of entomopathogenic nematodes by insect-damaged maize roots. Nature 434: 732-737

Röse U S R, Manukian A, Heath R R, Tumlinson J H (1996) Volatile semiochemicals released from undamaged cotton leaves. A systemic response of living plants to caterpillar damage. Plant Physiol 111: 487-495

Sabulal, B., Dan, M., Anil, J. J., Kurup, R. Pradeep, N. S., Valsamma, R. K. and George, V. (2006) Caryophyllene-rich rhizome oil of Zingiber nimmonii from South India: Chemical characterization and antimicrobial activity. Phytochemistry 67, 2469-2473

Schnee C, Köllner T G, Held M, Turlings T C J, Gershenzon J, Degenhardt J (2006) The products of a single maize sesquiterpene synthase form a volatile defense signal that attracts natural enemies of maize herbivores. Proc. Natl. Acad. Sci. USA 103(4): 1129-1134

Shen, B., Zheng, Z., and Dooner, H. K. (2001) A maize sesquiterpene cyclase gene induced by insect herbivory and volicitin: Characterization of wild-type and mutant alleles. Proc. Natl. Acad. Sci. USA 97, 14801-14806

Sotelo, A. (1997) [Need title]. In Functionality of Food Phytochemicals, T. Jones and J. Romeo, eds, (New York: Plenum Press), pp. 89-111

Starks, C. M., Back, K., Chappell, J., and Noel, J. P. (1997) Structural basis for cyclic terpene biosynthesis by tobacco 5-epi-aristolochene synthase. Science 277, 1815-1820.

Tholl, D., Chen, F., Petri, J., Gershenzon, J. and Pichersky, E. (2005) Two sesquiterpene synthases are responsible for the complex mixture of sesquiterpenes emitted from Arabidopsis flowers. The Plant Journal 42, 757-771

Trapp, S. C., and Croteau, R. (2001) Genomic organization of plant terpene synthases and molecular evolutionary implications. Genetics 158, 811-832

Turlings, T. C. J., Davison, A. and Tamo, C. (2004). A six-arm olfactometer permitting simultaneous observation of insect attraction and odour trapping. Physiological Entomology 29 (1): 45-55

Van de Peer, Y., and De Wachter, Y. (1994) TREECON for Windows: a software package for the construction and drawing of evolutionary trees for the Microsoft Windows environment. Computational Applications Biosciences 10, 569-570

Walter, M. H., Hans, J. and Strack, D. (2002) Two distantly related genes encoding 1-deoxy-D-xylulose 5-phosphate synthases: differential regulation in shoots and apocarotenoid-accumulating mycorrhizal roots. The Plant Journal 31, 243-254

Yoshikuni, Y., Ferrin, T. E. and Keasling, J. D. (2006) Designed divergent evolution of enzyme function. Nature 440, 1078-1082

1-58. (canceled)

59. A polynucleotide selected from the group consisting of:
(a) polynucleotides comprising a nucleotide sequence encoding a polypeptide having the amino acid sequence of SEQ ID NO:2 or 4;
(b) polynucleotides comprising the nucleotide sequence shown in SEQ ID NO:1 or 3;
(c) polynucleotides comprising a nucleotide sequence encoding a fragment of the polypeptide encoded by a polynucleotide of (a) or (b), wherein said nucleotide sequence encodes a protein having caryophyllene synthase activity;
(d) polynucleotides comprising a nucleotide sequence the complementary strand of which hybridizes to the polynucleotide of any one of (a) to (c), wherein said nucleotide sequence encodes a protein having caryophyllene synthase activity; and
(e) polynucleotides comprising a nucleotide sequence that deviates from the nucleotide sequence defined in (d) by the degeneracy of the genetic code.

60. A transgenic plant, which shows an increased activity of the polypeptide encoded by the polynucleotide of claim 59 compared to a corresponding wild-type plant.

61. A method for conferring resistance or increased resistance against a herbivore to a plant comprising the step of providing a transgenic plant in which the activity of the polypeptide encoded by the polynucleotide of claim 59 is increased as compared to a corresponding wild-type plant.

62. A regulatory sequence which comprises a DNA sequence selected from the group consisting of:
(a) the DNA sequence shown in SEQ ID NO:5 or 6;
(b) fragments of the DNA sequence of (a) being capable of mediating the transcription of a coding sequence operably linked thereto; and
(c) DNA sequences the complementary strand of which hybridizes to the DNA sequence of (a) or (b), wherein said DNA sequence is capable of mediating the transcription of a coding sequence operably linked thereto.

63. A polynucleotide having a length of at least 15 nucleotides which specifically hybridizes with a polynucleotide of claim 59 or with the complementary strand thereof.

64. A vector comprising the polynucleotide of claim 59.

65. A host cell which is genetically engineered with the polynucleotide of claim 59.

66. A transgenic plant cell which is genetically engineered with the polynucleotide of claim 59.

67. A transgenic plant or plant tissue comprising the plant cells of claim 66.

68. Propagation material or harvestable parts of the transgenic plant of claim 67.

69. Use of the polynucleotide of claim 59, for establishing or enhancing resistance against a herbivore in plants.

70. A method for breeding a plant having a resistance against a herbivore, said method comprising crossing and/or selfing progenitor lines and selecting for desirable traits, wherein said method is characterized by a selection step for progenitor lines and/or offspring that is/are capable of expressing the polypeptide encoded by the polynucleotide of claim 59.

71. A plant obtainable by the method of claim 70.

72. Use of the polynucleotide of claim 59 or of the regulatory sequence of claim 4 for selecting a plant having a resistance against a herbivore.

73. A kit comprising the polynucleotide of claim 63.

74. Use of the transgenic plant of claim 67 together with entomopathogenic nematodes for growing said plant.


SEQUENCE LISTING (26 sequences)

SEQ ID NO: 1 - DNA, 1644 bp, Zea mays
  CDS (1)..(1641)
atggcagctgatgaggcaagatccgtcagcaggctccacagtgaggaggacatgcatgggaagcaccactccactctatggggtgacttcttccttcaccatgtcccatgtcgtccaggacagtatttgattatgaaggataacgtcgaaataatgaaggaggaggtgaagaaaatgttactggatgttggatcctctgatttgtctcataagttagactgcattgatacactagaacggcttggactggattaccactataccaaggagattgatgaattaatgtgcaatgtcttcgaagctagggatcaagatcttgatcttactacaacatcacagcttttctatttgcttaggaagcatggatatcatatttcttctgatgtatttttgaagttcagagatgataaaggagatatcgtaacaaatgatgcaaggtgtttactgaggatgtacgaagcggcacatgttagagtgaacggtgaagaaatacttgacaatattctcattcacaccaagagacaacttcaatgtattgtagatgatctcgaaccaacactgcaagaagaagttagatatgcattggagacacctcttttcagaagactcaacagagtacaggccaggcaattcatctcaacatacgagaaaagcactacgcgtatcaatatgctcctggagttctcaaaattagacttcaacatcttgctcaccctatactgtgaggagctaaaagatcttacgctgtggtggaaagagttccaagcacaggctaacacgacaatctatgcacgagataggatggtggagatgcatttctggatgatgggagtcttctttgagccccaatactcctattcacgaaagatgctcacgcagttgtttatgatcgtgtctgtactcgatgacttgtatgacagccattgcaccacagaggaaggcaacgccttcactgcagcattacaaaggtgggatgaagaaggcgtggagcaatgcccaacatacttgaggactctgtatacaaatatacgtgccaccataaaagccatcgaggaggacttgaattttcaaaacaacaaacatgcaaagttggtaaaaggactgataatcgacatggccatgtgctataatgccgagacagaatggcgtgacaagaaatatgtgccagccaccgttgatgaacatctgaaaatttcagcacgcagcagtggctgtatgcacttggtgagccaggggttcatttcgatgggagatgtggcaactagcgaggccctcgagtgggcctctacctatcccaaaatcgttcgagctgtctgtatcattgcacgcctcgctaatgatataatgtcgtacaagcgagaagcatcaaacaatactatggtgtctacagtgcaaacttgtgcaaaagaatacggcaccaccacagtagaacaggccatagaaaagatcagagagctgatcgaggaagcgtggatggacatcacccatgagtgcctcagacagccacagccgaaggctcttctagagagggcggtcaacctggctcgtacaatggattttctgtacaaagatgcggatggatacacagattcacggagtatcaaaggcatattggattcattgtatgtgcatctaatagactaa

SEQ ID NO: 2 - protein, 547 aa, Zea mays
MAADEARSVSRLHSEEDMHGKHHSTLWGDFFLHHVPCRPGQYLIMKDNVEIMKEEVKKMLLDVGSSDLSHKLDCIDTLERLGLDYHYTKEIDELMCNVFEARDQDLDLTTTSQLFYLLRKHGYHISSDVFLKFRDDKGDIVTNDARCLLRMYEAAHVRVNGEEILDNILIHTKRQLQCIVDDLEPTLQEEVRYALETPLFRRLNRVQARQFISTYEKSTTRINMLLEFSKLDFNILLTLYCEELKDLTLWWKEFQAQANTTIYARDRMVEMHFWMMGVFFEPQYSYSRKMLTQLFMIVSVLDDLYDSHCTTEEGNAFTAALQRWDEEGVEQCPTYLRTLYTNIRATIKAIEEDLNFQNNKHAKLVKGLIIDMAMCYNAETEWRDKKYVPATVDEHLKISARSSGCMHLVSQGFISMGDVATSEALEWASTYPKIVRAVCIIARLANDIMSYKREASNNTMVSTVQTCAKEYGTTTVEQAIEKIRELIEEAWMDITHECLRQPQPKALLERAVNLARTMDFLYKDADGYTDSRSIKGILDSLYVHLID

SEQ ID NO: 3 - DNA, 1644 bp, Zea mays
  CDS (1)..(1641)
atggcagctgatgaggcaagatccgtcagcaggctccacagtgaggaggacatgcatgggaagcaccactccactctatggggtgacttcttccttcaccatgtcccatgtcgtccaggacagtatttgattatgaaggataacgtcgaaataatgaaggaggaggtgaagaaaatgttactggatgttggatcctctgatttgtctcataagttagactgcattgatacactagaacggcttggactggattaccactataccaaggagattgatgaattaatgtgcaatgtcttcgaagctagggatcaagatcttgatcttactacaacatcacagcttttctatttgcttaggaagcatggatatcatatttcttctgatgtatttttgaagttcagagatgataaaggagatatcgtaacaaaggatgcaaggtgtttactgaggatgtacgaagcggcacatgttagagtgaacggtgaagaaatacttgacaatattctcattcacaccaagagacaacttcaatgtattgtagatgatctcgaaccaacactgcaagaagaagttagatatgcattggagacacctcttttcagaagactcaacagagtacaggccaggcaattcatctcaacatacgagaaaagcactacgcgtatcaatatgctcctggagttctcaaaattagacttcaacatcttgctcaccctatactgtgaggagctaaaagatcttacgctgtggtggaaagagttccaagcacaggctaacacgacaatctatgcacgagataggatggtggagatgcatttctggatgatgggagtcttctttgagccccaatactcctattcacgaaagatgctcacgcagttgtttatgatcgtgtctgtactcgatgacttgtatgacagccattgcaccacagaggaaggcaacgccttcactgcagcattacaaaggtgggatgaagaaggcgtggagcaatgcccaacatacttgaggactctgtatacaaatatacgtgccaccataaaagccatcgaggaggacttgaattgtcaaaacaacaaacatgcaaagttggtaaaaggactgataatcgacatggccatgtgctataatgccgagacagaatggcgtgacaagaaatatgtgccagccaccgttgacgaacatctgaaaatttcagcacgcagcagtggctgtatgcacttggtgagccaggggttcatttcgatgggagatgtggcaactagcgaggccctcgagtgggcctctacctatcccaaaatcgttcgagctgtctgtatcattgcacgcctcgctaatgatataatgtcgtacaagcgagaagcatcaaacaatactatggtgtctacagtgcaaacttgtgcaaaagaatacggcaccaccacagtagaacaggccatagaaaagatcagagagctgatcgaggaagcgtggatggacatcacccatgagtgcctcagacagccacagccgaaggctcttctagagagggcggtcaacctggctcgtacaatggattttctgtacaaagatgcggatggatacacagattcacggagtatcaaaggcatattggattcattgtatgtgcatctaatagactaa

SEQ ID NO: 4 - protein, 547 aa, Zea mays
MAADEARSVSRLHSEEDMHGKHHSTLWGDFFLHHVPCRPGQYLIMKDNVEIMKEEVKKMLLDVGSSDLSHKLDCIDTLERLGLDYHYTKEIDELMCNVFEARDQDLDLTTTSQLFYLLRKHGYHISSDVFLKFRDDKGDIVTKDARCLLRMYEAAHVRVNGEEILDNILIHTKRQLQCIVDDLEPTLQEEVRYALETPLFRRLNRVQARQFISTYEKSTTRINMLLEFSKLDFNILLTLYCEELKDLTLWWKEFQAQANTTIYARDRMVEMHFWMMGVFFEPQYSYSRKMLTQLFMIVSVLDDLYDSHCTTEEGNAFTAALQRWDEEGVEQCPTYLRTLYTNIRATIKAIEEDLNCQNNKHAKLVKGLIIDMAMCYNAETEWRDKKYVPATVDEHLKISARSSGCMHLVSQGFISMGDVATSEALEWASTYPKIVRAVCIIARLANDIMSYKREASNNTMVSTVQTCAKEYGTTTVEQAIEKIRELIEEAWMDITHECLRQPQPKALLERAVNLARTMDFLYKDADGYTDSRSIKGILDSLYVHLID

SEQ ID NO: 5 - DNA, 1790 bp, Zea mays
aaccaccaaaattaatactgggaaaaggttaaccttatttccctttcaattagtctacccattttcatgatgtccttcttcgaaatcctaggtggtgtactacagaagctagatgctattcggtctagattcttttggcaaggaggacatcataaaaataaatatagactaggcaaatggaaactcatttgtcaacctaaagagctaggtgggctgggagtcgctaatcttgacataaagaacatttgtttactcagcacatagctttttaaattactcaatgaagatggtgtgtggcaacgtttattaagaaacaaatacttgggagacgaatccctaacacaagctgtcagaaaaccaggtgattatcatttctgggctagtctaatgaacatcaaggacaaattcctcaagtggggatattttaaggtgggaaacggacaagcaacaagattctgggaagataaatggcttaataattctcctcttagagagtaattcccaaacctttttaatatagtgcgcaataagacctcgctggttgcagaagtgctgtcacatgaaaatctaactctttctttccggagatccatggttggaattaagcttgtagaatggcagaacatgatacacttactggctacggttaatctgactccggtgagggacactttcgtgtggggagctcataggaatgggtctttctcaacctgatccatgtactataatttaattagtatcctcatttataatcgtaacggtgtgctttggaaattgaaattaccccttaatattaaaatctttctttcgtacctaggtaaggaggtgatcctaaccaaagacaatttaggcaaacgtagatggcgaggaagtctgaagtgcagtttttgtaatcagaacgagaacatacaacattttttctttgattactacatagctaaaactgtttggagaattatttattttactttaaaaatagactcacccgttaatattaatcatattataggggattgagaatccaacaaaggttgtatgcacaaaaggctgcttctttctagcgttgcggctttgttttggtcgatttggctttgtcgtaatgatgtggtgtttaatcataaccatataccgtcaactatacatgtgatctttagaggtacatattgttcacattttagagactcctgcagatggaagaaacacaacagcaaatcttagtcgtgtatcagactttggaggttatggccatggagttcttcacgagtcacgggtggcggtctaatgcaaggatcgatgccgtttagtgtttatgatcttttattccatatcatgttttaagttgaagtttgtttgatccatgaattcctgggatgatgtaacatttgaggtgtatggttgtatgcatgaaaatgcaaaagtccggaacgatatttccatttcaaaaaaagtctactggacggccaaaccaacattgttgagtggacgacctacgatattattaacaattccatgtgttgcacatgcctgtcggcagatctatatttaaaaaaaaacgagtggactacgacgctcagatccttccaggaaaaaaaacaatataatgcaaattgagtacgtacgtgcccacgtgcatcacgagtaccactccacgctggctacctatattaagaacaccataggatccaggtgcttgccagtgcatgcattgtatatctgcactgctacgccggtgggcacgtacgtgcaacaggcagcaggagagtagctagctagtatactatactttcatg

SEQ ID NO: 6 - DNA, 1789 bp, Zea mays
aaccaccaaaattaatactgggaaaaggttaaccttatttccctttcaattagtctacccattttcatgatgtccttcttcgaaatcctaggtggtgtactacagaagctagatgctattcggtccagattcttttggcaaggaggacatcataaaaataaatatagactaggcaaatggaaactcatttgtcaacctaaagagctaggtgggctgggagtcgctaatcttgacataaagaacatttgtttactcagcaaatagctttttaaattactcaatgaagatggtgtgtggcaacgtttattaagaaacaaatacttgggagacgaatccctaacacaagctgtcagaaaaccaggtgattatcatttctgggctagtctaatgaacatcaaggacaaattcctcaagtggagatattttaaggtgggaaacggacaagcaacaagattctgggaagataaatggcttaataattctcctcttagagagtaattcccaaacctttttaatatagtgcgcaataagacctcgctggttgcagaagtgctgtcacatgaaaatctaattctttgtttccggatatccatggttggaattaagcttgtagaatggcagaacatgatacacttactggctacggttaatctgactccggtgagggacactttcgtgtggggagctcataggaatgggtctttctcaacccgatccatgtactataatttaattagtatcctcatttataatcgtaacggtgtgctttggaaattgaaattaccccttaatattaaaatctttctttcgtacctaggtaaggaggtgatcctaaccaaagacaatttaggcaaacgtagatggcgaggaagtctgaagtgcagtttttgtaatcagaacgagaacatacaacattttttctttgattactacatagctaaaactgtttggagaattatttattttactttaaaaatagacccacccgttaatattaatcatattataggggattgagaatccaacaaaggttgtatgcacaaaaggctgcttctttctagcgttgcggctttgttttggtcgatttggctttgtcgtaatgatgtggtgtttaatcataaccatataccgtcaattatacatgtgatctttagaggtacatattgttcagattttagagactcctgcagaaggaagaaacacaacagcaaatcttagtcgtgtatcagactttggaggttatggccatggagttcttcacgagtcacgggtggcggtctaatgcaaggatcgatgacgtttagtgtttatgatcttttattccatatcatgttttaagttgaagtttgtttgatccataaattcctgggatgatgtaacatttgaggtgtatggttgtattcatgaaaatgcaaaagtccggaacgatatttccatttccaaaaaagtctactggacggccaaaccaacattgttgagtggacgacctacgatattattaacaattccatgtgttgcacatgcctgtcggcagatctatatttaaaaaaaacgagtggactacgacgctcagatccttccaggaaaaaaaacaatataatgcaaattgagtacgtacgtgcccacgtgcatcacgagtaccactccacgctggctacctatattaagaacaccataggatccaggtgcttgccagtgcatgcattgtatatctgcgctgctacgccggtgggcacgtacgtgcaacaggcagcaggagagtagctagctagtatactatactttcatg

SEQ ID NO: 7 - DNA, 2284 bp, Zea mays
gacgtacctgagtttgtcccttaaatcccgggtgtgacaagaaaatacaggcaaccaagtagaacttgttcatgcttaataggttattaagtgatttgcacgagaagtctggccgtagtgtgtgtttattttagttgtacaaaaatgaataagtcaaaattctaaggtatttaatttttaaatagaaaaattaatgtagcgaaacaaaaaataatcactttttgattgttgatgcttgaaaatggaaaaagtattgtgtggaacacactaagaaataactcaattcacgtgccacgtttttagagactttgcctcaggtttccatataaaaaagccacactaatataatttccaaagttttggcgcttggtaagggagattttataattatattcctttaggaggttgggtccaaccctaggagatttcacccattaagggcttattcgttttgctcaattcatgtggattgggtaggattgagtgggtttcaatcccaaataagtcaaaatcttttatttttttaatcccatccaatccacgtgccatagaaataaccgaacaaaccctaacgtagcatgtagaccggtcggtatcgaatggtctagtctcaatgacagtyggttctaccgatcaattggtggacctaccaactgatcgatacggttggtaccgatcagtggagcaattggtactaaccactgttttggatggtttgcttagtaccaacccaaatcgattgatactgattggatttagcaacatgtcttgttataattggtctacacacgtgtcacgtcatctacctgtttttaaactatttgaagtctaaaaaaacaaaagtgttatttataaattatgtaatttttagyttcacaaatcatgtaatttttatatttgcggtttatgaattatatagttttaattatggtttctgaatcctgtaaattttatttgcttaattttgtactcagatgaatctttatttcaaaacgttgtgtttcctttcaaataaaagattataaaagaaaaacggatgtggcgttgagccgttgaggtgactataggttatgataaacattgtgtactagagcattgtggtagataaaactgactatgattgatagcagggcaagtggcacggtctataatggacgtggcagtagataagaataaccgactacacgaaaaaagccgaaaaaagatatctggaatatatcagcatgataccttttgggaaacaaaatacatttgcaaatactacaaattaactagatacacacgggtatattgaattgttcatcctgtgggttatatagccaacagtgttttccgaactattttagtctattagatgcacatacaatgaatccaatatgcctttgatactccgtgaatctgtgtatccatccgcatctttgtacagaaaatccattgtacgagccaggttgaccgccctctctagaagagccttcggctgtggctgtctgaggcactcatgggtgatgtccatccacgcttcctcgatcagctctctgatcttttctatggcctgttctactgtggtggtgccgtattcttttgcacaagtttgcactgtagacaccatagtattgtttgatgcttctcgctgcagataaaaaagattcttcaaaaaattaattaattagaagatatatatatatatatatatatatacacatatatatggagtaattattgagttgcaccttgtacgacattatatcattagcgaggcgtgcaatgatacagacagctcgaacgattttgggataggtagaggcccactcgagggcctcgctagttgccacatctcccatcgaaatgaacccctggctcaccaagtgcatacagccactgctgcgtgctgaaattttcagatgttcatcaacggtggctggcacatatttcttgtcacgccattctgtctcggcattatagcacatggccatgtcgattatctaagatgcatgtgaaattagtgtcaaataagctcaataactttataatctaactatatactgttcgttagcaaatgtcattaattttgtcccgaacacaataacttctttttataatctactacacatgaaaagaaaatttgttagaactagattagtttagatgttgcacacttgtttgattagcatataataggtaaaaataaagcagtgacaaccatattgctttttttgtccaaatttaaatggataacaatgaatctagacacgtatataataatacacatacatcaaatattgca

SEQ ID NO: 8 - DNA, 920 bp, Zea mays
catatttatttggcccacccttaatcaaaattgattatatcctcctataaggtctgaaacaactagatctctctttttctttctcccttcgtcatcttctctagccactttccaactattttcaaaatataatgtaataccaatatagatattacttttatgttattagatatatagaatcaatcaaacaacccaattaatgtgatgcacacaactagcacctctttttcttttacaccctacacatatttataaggcagttgacatatatagagggttttttctagctgatttcacgctatgctatgaatttgttcatattggtttctctagttaattcacactggcctccatcaacaatcaggtgtctcttcaagtaaaatatatgactcgtactgtgttgtgcaaatgaaaggtttacactatagacaataagagaataaccaaattagatgaaatatctttcttgtagtatttgattatgaaggataacgtcgaaataatgaaggaggaggtgaagaaaatgttactggatgttggatcctctgatttgtctcataagttagactgcattgatacactagaacggcttggactggattaccactataccaaggagattgatgaattaatgtgcaatgtcttcgaagctagggatcaagatcttgatcttactacaacatcacagcttttctatttgcttaggaagcatggatatcatatttcttctggtaagcttgcagacatgttttgctattatagttggcgtatatttattagttggaacttcaaataacattaggactttttttggtctacatcttccacccaagcaacaaaggggtttttttactacaaatatattgtggcgatatatatatatatatatatatatatatatatatatatatataatatat

SEQ ID NO: 9 - DNA, 22649 bp, Zea mays
  misc_feature (16321)..(16321)
  misc_feature (16321)..(16321): n is a, c, g, or t
gatcgtccttcttatcgtcgttgtgagccctgccaccttccgcaggtggcccggccttgtgaaagtggcgccgaagcatggcgcactccccaagggtgtgcttgacgggcccctgatgataggggcacgactccttgagcatcttgtcgaagagattggcgcctccgggaggtttctgagggttcttgtactcagcggcggcgacaaggtccgcgtcggcggcgtcgcgtttcgcttgcgacttcttcttgcctttcttcttggtgtcgctctgagtggacgcctcggggacatctttcggctggcggccctggggctgcttgtccttccggaagatggcctcaaccgcttcctggccagaggcgaacttggtggcgatgtccatcagctcgctcgccctggtgggggtcttgcgacccagcttgctcaccaggtcgcggcaggtggtgccggcgaggaacgcgccgatgacgtccaagtcggtgacgttaggtagctcggtgcgctgcttcgagaatcgccggatgtagtcccggagagactctcccggctgctggcggcagcttcggagatcccaggagttcccagggcgcacgtacgtgccctggaagttgccgacgaaggcttggaccaggtcgtcccagttggagatctgccccggaggcaggtgctccaaccaggctcgagcggtgtcggagaggaatagggggaggttgcggatgatgaggttgtcatcgtccgttccacccagttggcaggccagccggtagtccgcgagccacagttccggcctcatctcccccgagtactttgtgatggtagtcggggttcggaaccgggttgggaacggcgcccgtcgtatggcccagctgaaagcctgcggaccgggtggttcgggcgagggactccgatcctccccgctgtcgtagcgtcccccacacctggggtggtagcctcggcgcaccctctcgtcgaggtgggttcgacggttgcggtgatggtgctcgttgccgaggcgacccggggccgcaggcgctgtgttgcgcgtgcgcccggtgtggaccgaggcttcccgcatgaaccgggaagtcgcggtgcgatgctccgaggggtacccctgccttcgggaggcagagctttcggcccgtcggaccgcgacatcctccaggagattcttgagctctccctggatacgccgcccctcggtggttgatggctccggcatcgcgcggagaagtattgccgctgcagccaggttctggccgactccactagaaaccggtggtggccttgccctgacatcgtcggcgatgcggtgctggatgccctggggtagatgacgcgcttctccggccggagcttggcccgcccatttccgcccgatgtcccggcggaacggctcaagtgttcctgctccctcgtcgagcctggcctgcatctcgcggatttgctcgagctgtgtgtcctgaccccccgcaaggactgggaccacagctagctctcaaaggatgtcaacgcgaggcgcaggcctagggggatcaccgttttccggtataccaagatggttgccttcgacgggaccccctagatcgacgtggaaacattcacgacttgggccgcagtcctcgtcgccgaggctgcggctaccgtcggaacagtcggaaaggcagtagtcgcatgcggtcatgaagtcctgcatggcactggggttaccaagtccggagaaatcccaacaaaattcgggctcgtcatcttccttggaatccgagggcccataggtcgagacggctgtcagccggtctcagggtgaccgcataccgtaccccggagggtttggacatgcctctatgaaggcttccaccgaagcgaagtcgcttggtgggtcgaggctgaatccagaaggcacgagatgggaatcggtcggtacctcctggtcgacgggcggtgacgaagtcgcgtcgggggcagactacaccgtcgtctcgggtacgagggtgacgcccaacaagtcctttgcgagcgtgctggcatcgtccgaccgcttggagttggcgtgtcgcggggaaacgacgctcgtcttcgtctcatactcgagggcgacgctcgacgtgtcccccgttggggcgccgacgccgtcgactcgctcgatagccgacgaggtgccgcctcctgcttggccttggttgccccgcctcctcctccgtcggcgggggaggcgacgggacaaacccgaatgttgctcttccaccacgtggggaagacgtcgtcgattccgccgccggcgggcgggtcgtcggccgccattatcgctgtcgcgcggcggagaaaggagtatcatgttgtagctcccgtcgagggacatgaactcgagactcccaaaacgaagcaccgtcccaggctggaaaggttgctggagactacccatctagagcttgacgggaagctgttcgtcaacacgcagcaggcccctacccggcgcgccaactgtcggtgtttcgaccccggggggtccctggaccgacgagtaaattgtcgccgcgtgtcccagcccagatggggcggcgcgagacggagcacggaggggggaaacaaggagacgggcgtaaaggggaaacccgcggccttcgtgtttgtccagcgcccaggtcgggtgcgcttgcagtagggggttacaagcgtccgcgtgggagggagcgagcggcctgcacgcgtcgtcccgtcctccccgcgtggccaaccttctgtacgagtgccctaggccttccttttataggcttaaggagagggcccaggtgtacaataggagatgtagcagtgtgctaacgtgtctagcagagaggagctagcgccctaagtacatgccgtcgtggcagccggagaggttttggcaccctgttcttgtgatgtcgtggccgtcggaggagcgccggagccttgcggaaggacagctgtcggggctgtcgagtccttgctgacgtctccttgcttccgtaaggggttgagagccgccgtcgtcatggagcacgcggggcgccatcattacttgtttaccggggcgagccagatgggacgccggtcttgttccccgtagcctgagctagctaggggtagggtaatggtggccccccctgtgatgtggtcggtccgagccctgggtcgggcgaggcggaggcttctccgaggttgaggtcgagactgtcttccgaggtcgaggtcgagtccgagcctcgggtcgggcgaggcggagttcgttgtcttccgggctgaggccgagtccgagccctggggtcgggcgaggcggagttcgtcgtcttccggggccgaggccgagtccgagccctggggtcaggcgaggcggagtccgtcgtcttccggagccgaggccgagtccgagccctgggtcgggcgaggcagagttcgtcgtcttccggggccgaggccgagtccgagccctggggtcgggcgaggcggagtccgtcgtcttccggagccgaggccgagtccgagccctgggtcgggcgaggcagagttcgtcgtccgaggccgagacgggggctgagccctggggtcaggcgaggcggagcttcctatggcgcccgaagccggacttggctgttgtcagcctcactctgtcgagcggcacagcagttggagcgacgcaggcggcgctgtcctcttgtcaggcgggtcagtggagcggcgaagtgactgcggtcacttcggctctgtcgactgaggggcgcgcgtcaggataaggtgtcaggccatctttgcattaaatgcttctacgatacggtcggttggcgtggcgacctggccaaggttgcttcttggcgaagactgggcctcgggcgagccgaaggtgtgtccattgcttgagggggccctcgggcgagacgtgaatcctccggggtcggctgcccttgcccgaggctgggctcgggcgaggcgaggtcatgtcccttgagtggaccaagccttgacttagacgcacccatcaagcctttgcagctttgtgcctatgggggttaccagctgagattaggagtcttgggggtacccctaattatggtccccgacagtactcctccacttgaaggttcttcaactcggacccgttgtcgctccttatcttcttcaccttgagctcaaactcattttgagctctccttaggaagcgcttgagggtcccttgggtttcagatttatcctgcaaaaagaatacccaagtgaagcgggaaaaatcatcaactataacaagaccatacttacttcctcctatgcttagataggcgacgggtccgaagaggtccatatgaagcaactccaaaggtcttgatgtggtcatcacatttttggtatgatgagagcttcccacctgtttacctgcttgacaagctgcacaaggtctatcttttttgaaggttacgtttgttagacctatcacgtgttctccctttagaagtttgtaaaggttcttcatccccatatgtgctaagcggcgatgccacagccagcccatgctagtcttagcaattaaacatgcatctagaccgacctcctcttttgcaaaatcaactaagtagagtttgtcgtctaatacacccttaaaagctaatgaaccatcactccttctaaagacggacacatctacatttgtgaataagcaattatatcccatattacaaagctgacttacagacaataagttatatccaagcgactcaactaaaaacacattagaaatagagtgctcggatgaaatggcaatcttccctagtcctttgaccttgccttgattcccgtcaccgaatatgattgtcttgggaatccttgttcttgacgtaggaggtgaacatcttcttctcccccgtcatgtggtttgtgcatccgctgtcgataatccagcttgatcccccggatgcataaacctgcaaggcaatttaggcttgggtcttaggtacccaactcttgttgggtcctacaaggttagtaacaatggtcttagggacccaaatgcaagttttatctcccttgcattttgcccctaatttcctagcaatcaccttattatcctttctacaaattgcaaaggaagcattgcaagcatgataaattgtagaaggttcattagttttcctaggaacattagcaacatttttcctaggcatatgatgaatgacatttctcctaggcatatctctaccatgcacataggaagaacttgaagcaaacatggcatttgaaatcatacactcctatgaacatttctagaatatctcttatcaccataaatgaaagcatgattcctttgactactattagtcataggagccttccctttctccttggcgggaatgggagccttatgacttgtgaagttcttggcttccctcttgaagccaagcccatccttaattgaggggtgtctaccaatagtgtaggcatcccttgcaaattttagtttgtcagattcatttttgctagtcttaagttgggcattaagactagccacttcatcatttaactttgtaatagagactaggtgttcactacaagcatcaacattaaaatctttacacctattgcaaattacaacatgatctacacaagagttagatctatttgctacttctagtttagcatttaaatcatcattaacactttttaaactagaaatgttctcatggcaagtagatagttcacaagaaagcattcattccttttaacttctaaagcaagagatttttgtgcctctacaaatttatcatgttcttcatacaaaagatcctcttgcttttctaacaatctattcttatcattcaaggcatcaattaattcattaatcttatcaatcttagttctatctaagcctttgaacaaactagcataatctatttcatcatcgctagattcatcatcactagaagaagcataagtagtctttcaagtgtttaccttcttctcctttgccatgaggcaagtgtgatgctcgttggggaagagggacgatttgttgaaggccgaggctgcgagtccttcgttgtcggagtcggacgacgaacaatccaagtcccactccttgccaaggtgtgcctcacccttagccttcttgtaagccttcttcttttccctcttcttctcttgttcctggtcactatcattatcgggacaattagcgataaaatgaccaatcttaccacatttgaagcatgagcacttcccctttgtcttgttcttgttgggatgatccttgtgaccccttagcgccgtcttgaaacgcttgatgatgagggccatctcttcctcattgagtcccgccgcctcaacttgtgccaccttgctaggtagcacttccttgctccttgttgctttgagagcaacggtttgaggctcgtggattgggccattcaacgcatcatcaacgtatcttgcctccttgatcatcatccgcccgcttacaaacttcccaagtatttcttcgggcgtcatcttggtgtacctaggattctcacgaatattgtttacaggatgtggatcaaggacagtgaaagaccttagcattaggcggacgacgtcgtggtccgtccatcgcgtgcttccatagctccttattttgttgacaagggtcttgagccggttgtacgtttgggttggctcctctgccctgatcatcgcgaaccttcctagttcgccttccaccaactccatcttggtgagcatggtgacgtcgttcccctcatgcgagatcttgagggtgtgccagatctgcttggcgttatccaagccgctcaccttatggtattcatccctgcacaatgatgctagaagaacagtagtagcttgtgcatttttgtgaatttgctcattgataaatatgggactatcagtactatcaaattgcattccactttctactatctcccatatgcttggatggagagagaacaagtgactacgcattttgtgactccaaaatccgtagtcctctccgtcaaagtgaggaggtttaccaagtggaatggagagtaaatgagcattggtactttgaggaatacgagagtaatcgaaagaaaaattagagttaactgtcttctttttctcgtagtcattgtcgtcgttgtccttttgggaagaagaggactcgtcgctgtcgtcgtagtagacgatcttcttgatgcgccttttcttcttcccttccttcttctttcgactcgagccggagtcagtagctttgtcgtccctcggctcgttgaagatggtctccttctccttatcattgaccaccatcccctttcccttaggatccatcccttcgggcgattagtccctttcttgaagagaacgactctgataccaattgagagcacctagagggggggggtgaataggtgatcctgtaaaaacttgaaacttaatgccacaaaacttgattaggagtcagcacaataaagccaagtggctagagagaagtgcttgcaagacacgataacaacaagaaaatcaacacagataggcacagtggtttatcctatggttcggccaagttcaacacttgcctactccacgttgtggcgtcccaacggacgagggttgcaatcaacccctctcaagcggtccaaagacccacttgaataccacggtgttttgctttgtttactatatcccgcttgcgaggaatatccacaacttggagcctctcgcccttacaatttgatgttcacaaagaagcacagaagtaaggctgggatgagcaacgcacgcaagacacaaaatcagagcacaaacatgcacacaagtcacaactcgagctcacaacacaacccaaagagttctctactcaaatggagctctagttgctatcacaaagaatcgaatgcgcggaattggagtcttggtgcttaggaatgcttagagaatgcttggtctcttcctccatgcgcctaggggtcccttttatagccccaaggcagctaggagccattgagagcattctaggaaggcaattcttgccttctgtcgcctggcgcaccggccagtccggtgcaccaccggacactgtccggcgcggatttctttcctattttggcacagccgaccgttggatattcagagccattggcgcaccggacactgtccggtgcccccttctgaccgttggctctgccacgcgtcgcgcgcggattacgcggccgaccgttggcccggccgactgttggctcaccggacagtccggtgcatcatcggacagtctggtgcatcatcagatagtccggtgatttatagtcgtacgccgccgacgaaacccgagagcagctagttcgccagagccagcctggcgcaccggacactgtccggtgcaccaccggacagtccggtgcacccagactacacagagtcttggctgctcagccaagtcttttccaaattggtcttttcctgtttctagcacttagacacaatacattagtctccaaaacaatgtactaagtcttagaaacatacctttactcttgatttgcacttcttaagtctttggcacaaatattactcaaggcatttgtgtgtagcacttaatcaccaaaatcttatagaaatgtcccaagggtacatttccctttcacatatcactaggcatcaaggtactaatggtatatgatgactcgctagtagtcatcagccaggtgaacaaagactgggattgctccatagactcattggacaactactatgccacggtgcgaaagctggaggacaaattcgaaggtctagaattccatcacatggaaagggatcacaatgcagcagcagatgcactatcgaaactagggtctagtcgggcccaggttccacccagagtttctgttcaggaggtgcagcaaccaagcatcgcagcagggcacgctaaggaatgcaacacgttagaccaggtgaagatagaccccaatgactggagggagccgattatcagatacataaaaatgaggaggagccagacgataaggccacatcagagcacattgcaatgcaatcagctcactataccatcatagggggtctgctatacaggagaggtgcaggaggagtcctcatgagatgcattcactcagctacggggaggcaactgctggaggaaatccatgctgggcagtgtggaatacacgcaacatcaaagaccttcgtagaaaaggctttcaggtcatgattctattgaccaacagcaaagaaagatacagcggacttagtccagaagtgcgaagcatgctagttcctattaaagaaacaacacttacccgtccaacatctgcaaactatccctgtaatcgggccttttgcatgctgggggctggatatgatcaggccattcaagaaagctcagggaggatacactcatatgctcgtcgccatcgataaatttacaaagttgatagagtacaaaccaatcgcctctttgactttagcaaaagcactggagttcattcaataaataatgttcagattcaggatacccaataggatcatcacagacctgggatctaacttcactagctcagggttctttggtttctatgagcagaggagcatctagatcaagtatgcattagtggcacacccgagagctaacgggcaggtagaaagagccaacaggatgataattgatgcactcaagaagaaagtattcgacaagagtgaaaagctcataagcaaatggatcagggaactactgtatgtagtctggagtctacggacccaacctagccgggccctgcatgggaacactccattcttcatggtatacggatcagaagcagtgttgctagttgatttgatttttggagcacccaggttgacattcgaaagcatagcagaagcggaagcaaccagactagaggatgttgacatattagaagaagagcgtttgaatgtcgtcatccaatcggccaggtatcagcagactctgaggcgctaccacgacaaagcggtgcgacaccgatcctttgcagtaggagacttggtactctgtcgcgtattgacaggagaaggacgacacaaactatcattgccaatgataggcctttcatcgtcgcagaagtcactcggcctaggtcctaccggctcacccagatggacggaactcaagtaggaaactcatggaatattgagcatctcaagaaattttacccatagtgatacaacaaactccggttggtgagctacacattgtaagccagagatctcattatcaataaatcttttgtgttcgtccacttgcaaacaaaccctcaaatagtgatcggcttaagtcggaaacgccttaagctgacgcagctcgcatacctgacttagggtgattactatacactgctaacggagcaatcggcttaagtcagaaatgccttaagtcggttagacattctcacgcatacatgacttagggtgattactataccctgctaacggagtaatcaacttaagtcgaaaacgccttaagtcggtgcgacatgctcacgcatacctgacttggggtgattactatgctctgctatcggagcaatcgacctaagtcggaaacgccttaagtcggcgagacatgcacatgcatacatgacttaggatgattactataccctgctagcgcagcagccggcttaagtcggaacgccttaagtcggtgtgacgtgctctcgcttacataacttagggttactatgccccatcaatgcatcaatcggcttaagttggaagcgccttaagtcggagcaacatgatcaattagacaacaaagagcttagggcacgtaccataccctactcacactcaaagggagaataactcaacgctcaaaaaaccgctgaaaaatatcatctcaatgaaaaacgttttctgtttgaacaaatcaatgtttgctttcactctcctattactgtgcaggagcatcctcgcgaggaaccttatctacaggatcaccagaagtagaaggattactcgaagtagccgacacatcaagtacatcagctacaatatcattaggcatcacaaagtcaggcctctttattaggatccggccagcccgaatggccttatccatcgctttgtcacaccacgccttcatgatagaacggtcaacctctaagaccttgatatcactacgggcagcctgcgcatcgcgacgagcaacctctagctcctgcgaaacaagtttcttcgaggcatgaagctctttgatgatggaatcttttgaagacaatgacctgacaagtcgtgatacctcgttctgggagattttcaactcatctctatggcgctcgagctctgacttagtaaagcggtggctcctctccaaggtggtgagggagacgctagcatcacggtacaatttgtccaaacagttccgagaatcaagaagtttctgcatctcctcctacagataagaagaacttgtgggtaaagatgtgcaacctctgtagagtgtaaaactggtatatcagccgtgctcacggtcatgagcggctcggaccctcatatgagtaatttatggagttaaacttaatttgccagatgcattgcattatgtgttttattatattaatgatgtcttattttattgggttggtatacacttatacttagtaattgctaataaaatttgaccaacttattaaaataaatgctcagctataaccattatttgttgattagccttacactatacatgagccacatcataagtgagttcatgcacattattccccacacttgttgaacgatgaacttttgtgagctcactgttgcgatacccccccaggtgaagagcaggtggccccaaaggaggactactatgacaagttcgacgaggtctaggtagcatctcccagtaagcttatgacgccaagggataataatgatattagtttgttctatttattatcagttattttgtaagacattttgttatataataaagattgtgacattcgtctctatacaccgagtcattatatgtgttgatcttgtttggcgcacatatgagacgtacctgagtttgtcccttaaatccgggtgtgacaagaaaatacaggcaaccaagtagaacttgttcatgcttaataggttattaagtgatttgcacgagaagtctggccgtagtgtgtgtttattttagttgtacaaaaatgaataagtcaaaattctaaggtatttaatttttaaatagaaaaattaatgtagcgaaacaaaaaataatcactttttgattgttgatgcttgaaaatggaaaaagtattgtgtggaacacactaagaaataactcaattcacgtgccacgtttttagagactttgcctcaggtttccatataaaaaagccacactaatataatttccaaagttttggcgcttggtaagggagattttataattatattcctttaggaggttgggtccaaccctaggagatttcacccattaagggcttattcgttttgctcaattcatgtggattgggtaggattgagtgggtttcaatcccaaataagtcaaaatcttttatttttttaatcccatccaatccacgtgccatagaaataaccgaacaaaccctaacgtagcatgtagaccggtcggtatcgaatggtctagtctcaatgacagtcggttctaccgatcaattggtggacctaccaactgatcgatacggttggtaccgatcagtggagcaattggtactaaccactgttttggatggtttgcttagtaccaacccaaatcgattgatactgattggatttagcaacatgtcttgttataattggtctacacacgtgtcacgtcatctacctgtttttaaactatttgaagtctaaaaaaacaaaagtgttatttataaattatgtaatttttagcttcacaaatcatgtaatttttatatttgcggtttatgaattatatagttttaattatggtttctgaatcctgtaaattttatttgcttaattttgtactcagatgaatctttatttcaaaacgttgtgtttcctttcaaataaaagattataaaagaaaaacggatgtggcgttgagccgttgaggtgactataggttatgataaacattgtgtactagagcattgtggtagataaaactgactatgattgatagcagggcaagtggcacggtctataatggacgtggcagtagataagaataaccgactacacgaaaaaagccgaaaaaagatatctggaatatatcagcatgataccttttgggaaacaaaatacatttgcaaatactacaaattaactagatacacacgggtatattgaattgttcatcctgtgggttatatagccaacagtgttttccgaactattttagtctattagatgcacatacaatgaatccaatatgcctttgatactccgtgaatctgtgtatccatccgcatctttgtacagaaaatccattgtacgagccaggttgaccgccctctctagaagagccttcggctgtggctgtctgaggcactcatgggtgatgtccatccacgcttcctcgatcagctctctgatcttttctatggcctgttctactgtggtggtgccgtattcttttgcacaagtttgcactgtagacaccatagtattgtttgatgcttctcgctgcagataaaaaagattcttcaaaaaattaattaattagaagatatatatatatatatatatatatacacatatatatggagtaattattgagttgcaccttgtacgacattatatcattagcgaggcgtgcaatgatacagacagctcgaacgattttgggataggtagaggcccactcgagggcctcgctagttgccacatctcccatcgaaatgaacccctggctcaccaagtgcatacagccactgctgcgtgctgaaattttcagatgttcatcaacggtggctggcacatatttcttgtcacgccattctgtctcggcattatagcacatggccatgtcgattatctaagatgcatgtgaaattagtgtcaaataagctcaataactttataatctaactatatactgttcgttagcaaatgtcattaattttgtcccgaacacaataacttctttttataatctactacacatgaaaagaaaatttgttagaactagattagtttagatgttgcacacttgtttgattagcatataataggtaaaaataaagcagtgacaaccatattgctttttttgtccaaatttaaatggataacaatgaatctagacacgtatataataatacacatacatcaaatattgcatgaattgattaacaccctaaaacgaatactattttgggacggagaagtaccataataaaggttatagatgatgtcaaccacaaggatgtagaacgaaacatttgcacataatgtaaccataaatgcttcacgacatttacacataatgtaaccataattattgatttatttctcagttttaatcttgatgtttcaattgagacataccagtccttttaccaactttgcatgtttgttgttttgaaaattcaagtcctcctcgatggcttttatggtggcacgtatatttgtatacagagtcctcaagtatgttgggcattgctccacgccttcttcatcccacctgtcatgtatgaaacttattattgtattaccatgagtattagaaaaatagaacttttctagagagaggagctttccatacaaataaacaattaagactatataaatatatcactggaagcaagggtaacagagaggattttagaatttcccattaatcaattttaaatagtaagagattctagcttcttcagggctggtttggtgacgggagatttgaagggggtccctagggaaagaaacctcttgagtttcctcccctagagatctccaaatatatggtcaccaaatcagtactcaattctctccattacacttgctcctaaacaaagtcatttaagtcgcatgatgaaaatataaacatactgtgaataagtgttgcaaacctttgtaatgctgcagtgaaggcgttgccttcctctgtggtgcaatggctgtcatacaagtcatcgagtacagacacgatcataaacaactgcgtgagcatctttcgtgaataggagtattggggctcaaagaagactcccatcatccagaaatgcatctccaccatcctatctcgtgcatagattgtcgtgttagcctgtgcttggaactctttccaccacctgttaaatacaatgtaaatggtcttagtacaaaataatgtggaaatgtgagtgaactaattaatgtgggactttgagagaacttagtactcccttctatctttttttatttgtcacggtttagtacaaaaataaactaacgagcgacaaatattcaagaacggaagtagtacaagattggtatgctattaatgacggttcttacagcgtaagatcttttagctcctcacagtatagggtgagcaagatgttgaagtctaattttgagaactccaggagcatattgatacgcgtagtgcttttctcgtatgttgagatgaattgcctggcctgtactctgttgagtcttctgaaaagaggtgtctccaatgcatatctaacttcttcttgcagtgttggttcgagatcatctacaatacattgaagttgtctcttggtgtgaatgagaatattgtcaagtatttcttcaccgttcactctaacatgtgccgcttcgtacatcctcagtaaacaccttgcatcatttgttacgatatctcctttatcatctctgaacttcaaaaatacatctgcattcgacactaacaacatgtgagacatccacctatatatgaaacagttatacgaccgtagatgtggcggttatagaataataggacagatccattgttcaaccttcaacttgaaccttagttcgattttgaacctttattattttaaataagatagaaaccacctttgaaacaagctaaagagaagaccaaatttgcccaaaactttgacgattggagggtgtctagtacctgattttatagttcagggtttaaaattggaccgttgtgcatgtatgttctgtcctctttataaactaatgatactacaattagcatttagcgttatatatatatatatatatatatatatatatatatatatatatatatatatatatatatatatcgccacaatatatttgtagtaaaaaaacccctttgttgcttgggtggaagatgtagaccaaaaaaagtcctaatgttatttgaagttccaactaatanatatacgccaactataatagcaaaacatgtctgcaagcttaccagaagaaaatatgatatccatgcttcctaagcaaatagaaaagctgtgatgttgtagtaagatcaagatcttgatccctagcttcgaagacattgcacattaattcatcaatctccttggtatagtggtaatccagtccaagccgttctagtgtatcaatgcagtctaacttatgagacaaatcagaggatccaacatccagtaacattttcttcacctcctccttcattatttcgacgttatccttcataatcaaatactgcaagaaagatatttcatctaatttggttattctcttattgtctatagtgtaaacctttcatttgcacaacacagtacgagtcatatattttacttgaagagacacctggttgttgatggaggccagtgtgaattaactagagaaaccaatatgaacaaattcatagcatagcgtgaaatcagctagaaaaaaccctctatatatgtcaactgccttataaatatgtgtagggtgtaaaagaaaaagaggtgctagttgtgtgcatcacattaattgggttgtttgattgattctatatatctaataacataaaagtaatatctatattggtattacattatattttgaaaatagttggaaagtggctagagaagatgacggagggagagagaaaaagagagatctagttgtttcagaccttataggaggatataatcaattttgattaagggtgggccaaataaatatgatggcattcatggatcttgtagatcgcacccgttcagtatttaagtttgaaatttctctatatctagaggcatgtttggtatacctaaagtttagttttgagactaaagtttagttcgtaccctatttggttttaggaactaaaagtattcagaacgcattaaattgactcataagaagactaaaatatctcttaacattctctagtcactagtgcaactgaaataaataagaggcaacaagtgaaaataatatgttttagttacactttaagggactaaagactaaaacagtttagtcacacccgtttaaaaatttaggaactaaatgagactaaaataaaatggagggactagtgattaatcccttaaaccacacgggaccctaagaaatctcatctagatcccccaattattgcatgcttgtttagttctgtcagattaaggtaattggatccagctcgtactgttcgacccttttgcatctagtagtttcgttcttttgcttacctactaccctacaaggttcagataaaaaaatgtgtatgggcgatcaacattctgtccatttgtttaccaatgattaccaaatcacatccggatggcgattactgtagtagcattttgcaaacttgggacatgtttggaacaactcctccaatgaataatctatatcttgaattcaatatatagcagctccaatggtggagctaagatccacttggagaactgtttgccaacacaacagctttagatcaacatccacctaatggcatgaaactgactcttttgtccttgttgggttggccttatgtcattgtcgtctgtcatccattgttcctcgttaggcaaaaaaagtgacgcgcgtgggcgcacaggcggacatgaacgtggccactggatcgagaatgtggccacagttggatgtggagaggcagagggaagaagaagataacagcattggaggtgggcacaactgaacgctgcctcgttgggggcgtggcctcgctggagccgtgtggaacatttatattcttgcatgtgttatgtgagaagtataggagtgcaggagatttagtcccaccttactagttaaggtggagggaacccaacttaaataatcgagtactctctatattcttgcatgtgttgtatgaaaagtataaggaaaaacatacgcgctcgctcccttcacctcgtcatctttaatctcgtcaccatggctcgtttgtgtcctcgtcacgtcattgttggcaacaacgaaacaaaacaagcgtacaacaacattatgcatatatttagatctgttttattttttgattaaattaaaaccacatgttctattttttccaacatatatttgtcatgaaactactcatttcatgaaacttggcatggtatgtaataggtgctttatcgtattgtttggtctttctacacatttcccttttttcaatttttgagactattttttgttcatagggatttcgacaccttggctcacaaacagatgtgggaaacaagataacttagggtccgtttggcagggctccgccttgcccaaaaatagctctacctctggcttttcttgaaaaccaactattttaaagctgaagctatttcacaaattgtttggcagaaacgacttatctagagccagagccatttccctgtattaaagtgctagagccaaagccaaaaccggtgaagttactatttatggcttcttctccgcggtacaaaaatgactctaacttttaggtgcttttcacattaagtcgtttaaaaaaggtgtttggcagggctctagcttttctctagagaaaagctagagccgatgccctaccaaacatgtacttagataagactcccggacggaagtgctcatggggaaacagatgtccacaaaaatccctctcatagaatccactagctttgaacctagcccattatggtggagggctaccaagtcaatcatcggattcatcttacaccctttcttgaagatcgtttccacattagaaggacgtgtattcttcatctcatgattaggttgtctctagcgcatacggtaaaccaccccttacgtagaggaagctcatgatatcctagggaggggagggcaggagctggcgacgatgtcgcgagggaagggccgcggtcggcgacgggggaggggtggctgggtcgcatggtagagggatggggcagtgggattgcgagggaggggagatatatgagggatgaaagatgatctaaataacattgaccacataatgtgagtgtgtaagatagaggatggtatccaacaatctcaactgttagttttgaaggtgtgatatgtgtctgtgcaattcgtttgatggatttaggggaggttataggtatgaggtgatttggttagatgggttttgcaaagttaaaactagtgggttatataggcgtatagatagagtgtaatgttttctcctctatatgttctagcggtatcctacaaagtatcatctaaatataaaggatgttccttcatcctctctatagagattttctcttttctccgctatctattttatactttacacaacagagtaattaagaaaactaaaatatttgagagcatgataaatatgtctataatatatgtttttaatcgtataggaggagatatagagaacaccaatataaaggaatggaatatagggcaaaaaatcttttaaaagagtctataaatgagagagagaccgcgaatactgaggatatggttgaatatagtcttatataactattttgctaagatattagagacagaagtaataaccgagcactctcttttattgcaatataatgataatttggaatcatctaacaaactcatactactacatacttgtcgtggagggcatgggacatgatgaaggaagaagtcaccccagagtggagtggtacttgccatccatgtcctcactgtgtgagcctagcagggtagcgcagatatacatggcattggcaggcacctccatcctactatgttgttcttatagacagcgtggagtggtacatatgatgcaacatacgtacgcagttttgcattttaattattgtttttttttctggacgcagatatgcaagtatgcaacgtacacatacacgcagagctgccaacagacatgcaacacctgtagctaaatccagtgcgagaactacatcaatacgtttcatgttttgtcatcacagtagctttagatatccccacctaatggcattttgattggaactaatacctcttttgtccttgggtatatagtaggcctcatgtcactgtctgccgtctgtttcttcctcgttagacaaaaaaaagtgacccagctgaagcatcggggttttggatgtgcatgtggggttttggatgcgccgaaggtggacgcgacctcgctgttgccaggtgcctaagaggcctaaagttagcacccttgtcacactttaggggtgcctaagactatttatagcagtctatctaaaatttatctaaaaatattgttttatactgttctgtactgtagactatactgtttgtagagtagagtttaattatggataataatatgggtaaactgctggagatagcctaaaggcctaaagcaaggataacttgcgttaggcaatttgttctgttatgttgtggcgtgtgttgttatgtccaaatcgaacgttttattaggtgtggtgtaattttgtatggcaagcttaaaaaatcttgctaaactttttacggccatctaagctagcttactaaggaaccaaacactaatctatggaaccgcaatgtggctaacatcagactatgtcagttgggacacaccctttcatctgacagagtgcaggccatacatatgtgccattggtggcgttgacaccgttggcagctcgtggctgttgttggttcttggtgaagatattaacagctagcacgtttggtttgaggaatgaagatcattttctcgctcctcgtattttctcgctcctcgttttttgttttggtttttggaatggaataagttaatctatcaccatttcattcctcatatgctagtgtaattagtaataatgtgatgaatgagttcgttccactaaattagtagaatagactcatgagacagcacctcatctagaatatattgatttttcaaaccaaacactccctaaatctttaggggtggacattcttgccttgaagttaaatgcatcgcagtgtagaagctcaatccttctagctaccttatggatttctacaatgaggagtgtatcaatgcttgtaccgtattattagatgacatatatttctcatcaaactttttccccctcaaatcttttcatgattattcgtaagctattaagaaatatatttaattattccacaataatagaagacgtgctctctcaaaacacaagtgcatcgatgcaatagtactagtgttgatagagacaattaaatatatttcttggtctttaaaccataagtggttacgcactgtatattaggacagaaggatactttatcatattctttgttttttcaaggcttttccatatatttttttttcaaattttgatactagttttgttcatagggatttcgaccccttcccaaacagatgcgagatctaaggccgtgttttattcgcagcaaatttttggaatccggctatttccattacccgagactatctcgttcgacgctgataatggagttctctacacactagatatccgagatgtaaaatacacagtgtttgttttatcgtccagattctcatggcagattttttcaatatctcaccgaaacaaacacgtgctaaaatctaaagtatattatgtcgtctataatataagtaattaacgtatagagaaggtgatatagagaactttgtggagagaattgagatatagaggatgaaatcttttagatgaatctataaagatgagataatatggtcagagacattcgtacatataactattttgccaagatattagaggtagcaatagtacgtaataactgagcacactctcttttatttcaatataatggtaatttgggattatctcaagcaaaaaaactccagtaatgattaataacaaaagccggagaggatggatttgttcaacatactatacgtacctgtcctggacgacatgggacatggtgaaggaagaagtcaccccatagagtggagtggtgcttcccatgcatgtcctcctcactgtggagcctgctgacggatcttgcctcatcagctgccatgaaagtatagtatactagctagctactctcctgctgcctgttgcacgtacgtgcccaccggcgtagcagtgcagatatacaatgcatgcactggcaagcacctggatcctatggtgttcttaatataggtagccagcgtggagtggtactcgtgatgcacgtgggcacgtacgtactcaatttgcattatattgttttttttcctggaaggatctgagcgtcgtagtccactcgtttttttttaaatatagatc

SEQ ID NO: 10 - DNA, 21 bp, artificial sequence
  primer oligonucleotide
gaacttcaaaaatacatcaga

SEQ ID NO: 11 - DNA, 25 bp, Artificial
  primer oligonucleotide
gacggatcttgcctcatcagctgcc

SEQ ID NO: 12 - DNA, 25 bp, Artificial
  primer oligonucleotide
gtatactagctagctactctcctgc

SEQ ID NO: 13 - DNA, 24 bp, Artificial
  primer oligonucleotide
atggcagctgataggcaagatccg

SEQ ID NO: 14 - DNA, 26 bp, Artificial
  primer oligonucleotide
ttagtctattagatgcacatacaatg

SEQ ID NO: 15 - DNA, 24 bp, Artificial
  primer oligonucleotide
gttagtccaatatttgtgttgggc

SEQ ID NO: 16 - DNA, 25 bp, Artificial
  primer oligonucleotide
gacggatcttgcctcatcagctgcc

SEQ ID NO: 17 - DNA, 25 bp, Artificial
  primer oligonucleotide
ttcaaccaccaaaattaatactggg

SEQ ID NO: 18 - DNA, 25 bp, Artificial
  primer oligonucleotide
gtatactagctagctactctcctgc

SEQ ID NO: 19 - DNA, 34 bp, Artificial
  primer oligonucleotide
gtgctataatgccgagacagaatggcgtgacaag

SEQ ID NO: 20 - DNA, 36 bp, Artificial
  primer oligonucleotide
caattcatgtggattgggtaggattgagtgggtttc

SEQ ID NO: 21 - DNA, 24 bp, Artificial
  primer oligonucleotide
gatctaaggccgtgttttattcgc

SEQ ID NO: 22 - DNA, 24 bp, Artificial
  primer oligonucleotide
agtaacattttcttcacctcctcc

SEQ ID NO: 23 - DNA, 24 bp, Artificial
  primer oligonucleotide
cacagtgaggaggacatgcatggg

SEQ ID NO: 24 - DNA, 24 bp, Artificial
  primer oligonucleotide
atttcgacgttatccttcataatc

SEQ ID NO: 25 - DNA, 32 bp, Artificial
  primer oligonucleotide
attgccatggcgcagctgatgaggcaagatcc

SEQ ID NO: 26 - DNA, 32 bp, Artificial
  primer oligonucleotide
attagaattcttagtctattagatgcacatac